(19) United States
(12) Patent Application Publication
(10) Pub. No.: US 2019/0233883 A1
(43) Pub. Date: Aug. 1, 2019

(54) METHODS AND COMPOSITIONS FOR ANALYZING NUCLEIC ACID

(71) Applicant: Agena Bioscience, Inc., San Diego, CA (US)

(72) Inventor: Charles R. CANTOR, Del Mar, CA (US)

(21) Appl. No.: 16/378,104

(22) Filed: Apr. 8, 2019

Related U.S. Application Data
(63) Continuation of application No. 14/402,658, filed on Nov. 20, 2014.
(60) Provisional application No. 61/649,854, filed on May 21, 2012.

(51) Int. Cl. C12Q 1/6825; C12Q 1/68

(52) U.S. Cl. CPC C12Q 1/6825; C12Q 1/68

(57) ABSTRACT

Technology provided herein relates in part to methods, processes, compositions and apparatuses for analyzing nucleic acid.

### RELATED APPLICATIONS

This patent application is a continuation of U.S. Ser. No. 14/402,658, filed on Nov. 20, 2014 entitled METHODS AND COMPOSITIONS FOR ANALYZING NUCLEIC ACID, naming Charles R. Cantor as inventor, designated by attorney docket no.: AGB-6044-US, which is a 35 U.S.C. 371 national stage of international patent application number PCT/US2013/041354 filed on May 16, 2013, entitled METHODS AND COMPOSITIONS FOR ANALYZING NUCLEIC ACID, naming Charles R. CANTOR as inventor, designated by Attorney Docket No.: AGB-6044-PC, which claims the benefit of U.S. Provisional Patent Application No. 61/649,854 filed on May 21, 2012, entitled METHODS AND COMPOSITIONS FOR ANALYZING NUCLEIC ACID, naming Charles R. CANTOR as inventor, designated by Attorney Docket No.: AGB-6044-PV. The entire content of the foregoing patent applications is incorporated herein by reference, including all text, tables and drawings.

### FIELD

Technology provided herein relates in part to methods, processes, compositions and apparatuses for analyzing nucleic acid.

### BACKGROUND

Genetic information of living organisms (e.g., animals, plants and microorganisms) and other forms of replicating genetic information (e.g., viruses) is encoded in deoxyribonucleic acid (DNA) or ribonucleic acid (RNA). Genetic information is a succession of nucleotides or modified nucleotides representing the primary structure of chemical or hypothetical nucleic acids. In humans, the complete genome contains about 30,000 genes located on twenty-four (24) chromosomes (see The Human Genome, T. Strachan, BIOS Scientific Publishers, 1992). Each gene encodes a specific protein, which after expression via transcription and translation, fulfills a specific biochemical function within a living cell.

Many medical conditions are caused by one or more genetic variations. Certain genetic variations cause medical conditions that include, for example, hemophilia, thalassemia, Duchenne Muscular Dystrophy (DMD), Huntington's Disease (HD), Alzheimer's Disease and Cystic Fibrosis (CF) (Human Genome Mutations, D. N. Cooper and M. Krawczak, BIOS Publishers, 1993). Such genetic diseases can result from an addition, substitution, or deletion of a single nucleotide in DNA of a particular gene. Certain birth defects are caused by a chromosomal abnormality, also referred to as an aneuploidy, such as Trisomy 21 (Down's Syndrome), Trisomy 13 (Patau Syndrome), Trisomy 18 (Edward's Syndrome), Monosomy X (Turner's Syndrome) and certain sex chromosome aneuploidies such as Klinefelter's Syndrome (XXY), for example. Some genetic variations may predispose an individual to, or cause, any of a number of diseases such as, for example, diabetes, arteriosclerosis, obesity, various autoimmune diseases and cancer (e.g., colorectal, breast, ovarian, lung).

Identifying one or more genetic variations or variances can lead to diagnosis of, or determining predisposition to, a particular medical condition. Identifying a genetic variance can result in facilitating a medical decision and/or employing a helpful medical procedure. In some embodiments, identification of one or more genetic variations or variances involves the analysis of cell-free DNA. Cell-free DNA (CF-DNA) is composed of DNA fragments that originate from cell death and circulate in peripheral blood. High concentrations of CF-DNA can be indicative of certain clinical conditions such as cancer, trauma, burns, myocardial infarction, stroke, sepsis, infection, and other illnesses. Additionally, cell-free fetal DNA (CFF-DNA) can be detected in the maternal bloodstream and used for various noninvasive prenatal diagnostics.

The presence of fetal nucleic acid in maternal plasma allows for non-invasive prenatal diagnosis through the analysis of a maternal blood sample. For example, quantitative abnormalities of fetal DNA in maternal plasma can be associated with a number of pregnancy-associated disorders, including preeclampsia, preterm labor, antepartum hemorrhage, invasive placentation, fetal Down syndrome, and other fetal chromosomal aneuploidies. Hence, fetal nucleic acid analysis in maternal plasma is a useful mechanism for the monitoring of fetomaternal well-being.

Early detection of pregnancy-related conditions, including complications during pregnancy and genetic defects of the fetus is important, as it allows early medical intervention necessary for the safety of both the mother and the fetus. Prenatal diagnosis traditionally has been conducted using cells isolated from the fetus through procedures such as chorionic villus sampling (CVS) or amniocentesis. However, these conventional methods are invasive and present an appreciable risk to both the mother and the fetus. The National Health Service currently cites a miscarriage rate of between 1 and 2 percent following the invasive amniocentesis and chorionic villus sampling (CVS) tests. An alternative to these invasive approaches is the use of non-invasive screening techniques that utilize circulating CFF-DNA.

### SUMMARY

Provided in some aspects are compositions comprising four nucleotide species, where the nucleotide species have substantially identical separation properties when separated by a mass-sensitive process. Also provided, in some aspects, are methods for generating a complementary copy of a nucleic acid fragment comprising contacting under polymerization conditions a nucleic acid fragment with a composition comprising four nucleotide species, wherein the nucleotide species have substantially identical separation properties when separated by a mass-sensitive process, thereby generating a complementary copy of the nucleic acid fragment.

In some embodiments, polynucleotides having an equal total number of the nucleotide species have substantially identical separation properties when separated by a mass-sensitive process. In some embodiments, at least three of the nucleotide species are mass-modified. In some embodiments, the nucleotide species have substantially identical mass. In some embodiments, the nucleotide species each are capable of hybridizing to one of adenine, thymine, cytosine and guanine in a polynucleotide, where the adenine, thymine, cytosine and guanine are not mass-modified. In some embodiments, the nucleotide species are capable of forming phosphodiester bonds when polymerized. In some embodiments, each mass-modified nucleotide species comprises one or more mass modifiers. In some embodiments, each mass-modified nucleotide species comprises one or more isotopes, and in some embodiments, the one or more isotopes are one or more stable isotopes. In some embodiments, each mass-modified nucleotide species comprises one or more isotopes and one or more other mass modifiers. In some embodiments, the one or more isotopes comprise a hydrogen isotope. In some embodiments, the hydrogen isotope is deuterium. In some embodiments, the one or more isotopes comprise a nitrogen isotope. In some embodiments, the nitrogen isotope is nitrogen-15. In some embodiments, the one or more isotopes comprise an oxygen isotope. In some embodiments, the oxygen isotope is oxygen-17 or oxygen-18. In some embodiments, the one or more isotopes comprise a carbon isotope. In some embodiments, the carbon isotope is carbon-13.

Also provided, in some aspects, are methods for determining length of a nucleic acid fragment, comprising a) contacting, under annealing conditions, a nucleic acid fragment with a probe, which probe (i) comprises at least two nucleotide species which have substantially identical separation properties, and (ii) is longer than the nucleic acid fragment to which it anneals, thereby generating a fragment-probe species comprising one or more unhybridized probe portions; b) removing the one or more unhybridized probe portions from the fragment-probe species, thereby generating a trimmed probe; and c) determining the length of the trimmed probe, thereby determining the length of the nucleic acid fragment.

Also provided, in some aspects, are methods for determining lengths of nucleic acid fragments in a mixture of nucleic acid fragments having different lengths, comprising a) contacting, under annealing conditions, nucleic acid fragments with a plurality of probes, which probes: (i) comprise at least two nucleotide species which have substantially identical separation properties, and (ii) are longer than the nucleic acid fragments to which they anneal, thereby generating fragment-probe species comprising unhybridized probe portions; b) removing the unhybridized probe portions from the fragment-probe species, thereby generating trimmed probes; and c) determining lengths of the trimmed probes, thereby determining the lengths of the nucleic acid fragments.

Also provided, in some aspects, are methods for detecting the presence or absence of a genetic variation comprising (a) contacting under annealing conditions target fragments and reference fragments from a nucleic acid sample with a plurality of probes that can anneal to the fragments, which probes (1) comprise at least two nucleotide species which have substantially identical separation properties, and (2) are longer than the fragments to which they anneal, thereby generating target-probe species and reference-probe species comprising unhybridized probe portions; (b) separating the target-probe species reference-probe species from the nucleic acid sample; (c) removing the unhybridized probe portions of the target-probe species and the reference-probe species, thereby generating trimmed probes; (d) determining lengths of the trimmed probes, thereby determining the lengths of the target fragments and reference fragments; (e) quantifying the amount of at least one target fragment length species and at least one reference fragment length species; and (f) providing an outcome determinative of the presence or absence of a genetic variation from the quantification in (e), with the proviso that the outcome is provided without determining nucleotide sequences of the target fragments and the reference fragments.

Also provided, in some aspects, are methods for detecting the presence or absence of a genetic variation comprising (a) separating target fragments and reference fragments from a nucleic acid sample based on nucleotide sequences in the target fragments and the reference fragments and substantially not in other fragments in the sample, thereby generating separated fragments comprising separated target fragments and separated reference fragments; (b) determining lengths of the separated target fragments and separated reference fragments by a process comprising i) contacting under annealing conditions the separated fragments with a plurality of probes that can anneal to the separated fragments, which probes (1) comprise at least two nucleotide species which have substantially identical separation properties, and (2) are longer than the separated fragments to which they anneal, thereby generating target-probe species and reference-probe species comprising unhybridized probe portions; ii) removing the unhybridized probe portions of the target-probe species and the reference-probe species, thereby generating trimmed probes; and iii) determining lengths of the trimmed probes, thereby determining the lengths of the separated target fragments and separated reference fragments; (c) quantifying the amount of at least one separated target fragment length species and at least one separated reference fragment length species; and (d) providing an outcome determinative of the presence or absence of a genetic variation from the quantification in (c), with the proviso that the outcome is provided without determining nucleotide sequences of the target fragments and the reference fragments.

In some embodiments, the probe comprises at least one mass-modified nucleotide species. In some embodiments, the probe comprises at least two mass-modified nucleotide species. In some embodiments, the probe comprises at least three mass-modified nucleotide species. In some embodiments, the probe comprises at least four mass-modified nucleotide species. In some embodiments, the probe comprises at least three nucleotide species of substantially identical mass. In some embodiments, the probe comprises at least four nucleotide species of substantially identical mass. In some embodiments, all nucleotide species in the probe are of substantially identical mass. In some embodiments, the probes comprise a first set of nucleotide species having substantially identical mass and a second set of nucleotide species having substantially identical mass, where the mass of the first set is different than the mass of the second set. In some embodiments, nucleotide species of the first set are purines, derivatives thereof or combinations thereof and nucleotide species of the second set are pyrimidines, derivatives thereof or combinations thereof.

Also provided, in some aspects, are methods for determining length of a nucleic acid fragment, comprising a) generating a complementary copy of the nucleic acid fragment, which fragment copy comprises at least two nucleotide species which have substantially identical separation properties; and b) determining the length of the fragment copy, thereby determining the length of the nucleic acid fragment.

Also provided, in some aspects, are methods for determining length of a nucleic acid fragment, comprising a) ligating a priming site to the nucleic acid fragment, thereby generating a ligated nucleic acid fragment; b) contacting, under annealing conditions, the ligated nucleic acid fragment with a primer which is capable of hybridizing to the primer site in (a); c) extending the primer with a set of nucleotides, which set comprises at least two nucleotide species which have substantially identical separation properties, thereby generating a complementary copy of the fragment comprising modified nucleotides; and d) determining the length of the fragment copy, thereby determining the length of the nucleic acid fragment.

Also provided, in some aspects, are methods for determining lengths of nucleic acid fragments in a mixture of nucleic acid fragments having different lengths, comprising: a) ligating priming sites to the nucleic acid fragments, thereby generating ligated nucleic acid fragments; b) contacting, under annealing conditions, the ligated nucleic acid fragments with primers which are capable of hybridizing to the priming sites in (a); c) extending the primers with a set of nucleotides, which set comprises at least two nucleotide species which have substantially identical separation properties, thereby generating complementary copies of the fragments comprising modified nucleotides; and d) determining the lengths of the fragment copies, thereby determining the lengths of the nucleic acid fragments.

Also provided, in some aspects are methods for detecting the presence or absence of a genetic variation comprising (a) separating target fragments and reference fragments from a nucleic acid sample based on nucleotide sequences in the target fragments and the reference fragments and substantially not in other fragments in the sample, thereby generating separated fragments comprising separated target fragments and separated reference fragments; (b) determining lengths of the separated target fragments and separated reference fragments by a process comprising i) generating complementary copies of the of the separated target fragments and separated reference fragments, where each fragment copy comprises at least two nucleotide species which have substantially identical separation properties; and ii) determining the lengths of the fragment copies, thereby determining the lengths of the separated target fragments and separated reference fragments (c) quantifying the amount of at least one separated target fragment length species and at least one separated reference fragment length species; and (d) providing an outcome determinative of the presence or absence of a genetic variation from the quantification in (c). In some embodiments, the outcome is provided without determining nucleotide sequences of the target fragments and the reference fragments

In some embodiments, the fragment copy comprises at least one mass-modified nucleotide species. In some embodiments, the fragment copy comprises at least two mass-modified nucleotide species. In some embodiments, the fragment copy comprises at least three mass-modified nucleotide species. In some embodiments, the fragment copy comprises at least four mass-modified nucleotide species. In some embodiments, the fragment copy comprises at least three nucleotide species of substantially identical mass. In some embodiments, the fragment copy comprises at least four nucleotide species of substantially identical mass. In some embodiments, all nucleotide species in the fragment copy are of substantially identical mass. In some embodiments, the fragment copy comprises a first set of nucleotide species having substantially identical mass and a second set of nucleotide species having substantially identical mass, where the mass of the first set is different than the mass of the second set. In some embodiments, nucleotide species of the first set are purines, derivatives thereof or combinations thereof and nucleotide species of the second set are pyrimidines, derivatives thereof or combinations thereof.

In some embodiments, the mass-modified nucleotide species are joined by phosphodiester bonds in the probes or fragment copies. In some embodiments, the mass-modified nucleotide species are capable of polymerizing on a nucleic acid template. In some embodiments, each mass-modified nucleotide species is capable of hybridizing to one of adenine, thymine, cytosine and guanine in a polynucleotide, where the adenine, thymine, cytosine and guanine are not mass-modified.

In some embodiments, each mass-modified nucleotide species comprises one or more mass modifiers. In some embodiments, each mass-modified nucleotide species comprises one or more isotopes, and in some embodiments, the one or more isotopes are one or more stable isotopes. In some embodiments, each mass-modified nucleotide species comprises one or more isotopes and one or more other mass modifiers. In some embodiments, the one or more isotopes comprise a hydrogen isotope. In some embodiments, the hydrogen isotope is deuterium. In some embodiments, the one or more isotopes comprise a nitrogen isotope. In some embodiments, the nitrogen isotope is nitrogen-15. In some embodiments, the one or more isotopes comprise an oxygen isotope. In some embodiments, the oxygen isotope is oxygen-17 or oxygen-18. In some embodiments, the one or more isotopes comprise a carbon isotope. In some embodiments, the carbon isotope is carbon-13.

In some embodiments, determining the lengths of the trimmed probes or fragment copies comprises use of a mass sensitive process. In some embodiments, the mass sensitive process comprises mass spectrometry. In some embodiments, the mass sensitive process comprises electrophoresis. In some embodiments, the mass sensitive process does not comprise electrophoresis. In some embodiments, the nucleotide sequences of the nucleic acid fragments are not determined.

In some embodiments, the number of fragments in a sample is determined for at least one target fragment length species and at least one reference fragment length species. In some embodiments, the target fragments and reference fragments are separated using a selective nucleic acid capture process. In some embodiments, the selective nucleic acid capture process comprises use of a solid phase array.

In some embodiments, the method further comprises isolating the sample from a subject. In some embodiments, the sample is from a pregnant female. Sometimes the sample is blood, urine, saliva, a cervical swab, serum, and sometimes is plasma. In some embodiments, the method further comprises isolating nucleic acid from the sample. In some embodiments, the nucleic acid in the sample is circulating cell-free nucleic acid. In some embodiments, the target nucleic acid fragments are from chromosome 13. In some embodiments, the target nucleic acid fragments are from chromosome 18. In some embodiments, the target nucleic acid fragments are from chromosome 21. In some embodiments, the target nucleic acid fragments are from chromosome 13, chromosome 18 and/or chromosome 21. In some embodiments, the genetic variation is a fetal aneuploidy. Sometimes the fetal aneuploidy is trisomy 13. Sometimes the fetal aneuploidy is trisomy 18. Sometimes the fetal aneuploidy is trisomy 21.

In some embodiments, the method further comprises determining the fraction of fetal nucleic acid in the sample and providing the outcome based in part on the fraction.

Certain aspects of the technology are described further in the following description, examples, drawings and claims.

### BRIEF DESCRIPTION OF THE DRAWINGS

The drawings illustrate aspects of the technology and are not limiting. For clarity and ease of illustration, the drawings are not made to scale and, in some instances, various aspects may be shown exaggerated or enlarged to facilitate an understanding of particular embodiments.

FIG. 1 shows a method for determining nucleic acid fragment length, which includes the steps of 1) hybridization of probe (P; dotted line) to fragment (solid line), 2) trimming of the probe, and 3) measuring probe length. Fragment size determination is shown for a fetally-derived fragment (F) and a maternally-derived fragment (M).

FIG. 2 shows a method for determining nucleic acid fragment length, which includes the steps of 1) ligation of fragment to a universal primer site conjugated to a bead; 2) hybridization of universal primer to ligation product; 3) extension of the primer, thereby generating a copy of the nucleic acid fragment which comprises mass-modified nucleotides; 4) denaturation of the fragment-copy duplex and separation of the copy from the fragment; and 5) measurement of copy size using a mass-sensitive process.

### DETAILED DESCRIPTION

Provided herein are methods and compositions for analyzing nucleic acid which include, for example, methods for determining nucleic acid fragment length and methods for determining the presence or absence of a genetic variation. Determination of nucleic acid fragment length typically involves sequencing of the nucleic acid fragment or use of a mass-sensitive process. While certain sequencing methods can provide a fairly accurate assessment of fragment length, such methods can be expensive and time consuming. Measuring nucleic acid fragment size using a mass-sensitive method, such as mass spectrometry, can be a faster and cheaper approach for determining fragment length. However, nucleic acid fragments having different nucleotide sequences may have different nucleotide compositions (i.e., total number of each nucleotide species). Because each nucleotide species has a unique mass value, fragments having the same length but different nucleotide compositions may have different masses. Thus, fragment size determined by a mass sensitive process reflects both fragment length and nucleotide composition. Direct assessment of nucleic acid fragments using a mass sensitive process may only provide a range of possible lengths for each fragment. Indirect assessment of nucleic acid fragments, however, using probes comprised of nucleotides having similar or identical separation properties (e.g., identical masses) can provide accurate measurements of nucleic acid fragment length. Provided herein are compositions comprising nucleotide species having substantially identical separation properties when separated by a mass-sensitive process and methods for determining nucleic acid fragment length using such compositions.

Provided also are improved methods, processes and apparatuses useful for identifying genetic variations. Identifying one or more genetic variations or variances can lead to diagnosis of, or determining predisposition to, a particular medical condition. Identifying a genetic variance can result in facilitating a medical decision and/or employing a helpful medical procedure.

### Samples

Provided herein are methods and compositions for analyzing nucleic acid. In some embodiments, nucleic acid fragments in a mixture of nucleic acid fragments are analyzed. A mixture of nucleic acids can comprise two or more nucleic acid fragment species having different nucleotide sequences, different fragment lengths, different origins (e.g., genomic origins, fetal vs. maternal origins, cell or tissue origins, sample origins, subject origins, and the like), or combinations thereof.

Nucleic acid or a nucleic acid mixture utilized in methods and apparatuses described herein often is isolated from a sample obtained from a subject. A subject can be any living or non-living organism, including but not limited to a human, a non-human animal, a plant, a bacterium, a fungus or a protist. Any human or non-human animal can be selected, including but not limited to mammal, reptile, avian, amphibian, fish, ungulate, ruminant, bovine (e.g., cattle), equine (e.g., horse), caprine and ovine (e.g., sheep, goat), swine (e.g., pig), camelid (e.g., camel, llama, alpaca), monkey, ape (e.g., gorilla, chimpanzee), ursid (e.g., bear), poultry, dog, cat, mouse, rat, fish, dolphin, whale and shark. A subject may be a male or female (e.g., woman).

Nucleic acid may be isolated from any type of suitable biological specimen or sample. Non-limiting examples of specimens include fluid or tissue from a subject, including, without limitation, umbilical cord blood, chorionic villi, amniotic fluid, cerebrospinal fluid, spinal fluid, lavage fluid (e.g., bronchioalveolar, gastric, peritoneal, ductal, ear, arthroscopic), biopsy sample (e.g., from pre-implantation embryo), celocentesis sample, fetal nucleated cells or fetal cellular remnants, washings of female reproductive tract, urine, feces, sputum, saliva, nasal mucous, prostate fluid, lavage, semen, lymphatic fluid, bile, tears, sweat, breast milk, breast fluid, embryonic cells and fetal cells (e.g. placental cells). In some embodiments, a biological sample is a cervical swab from a subject. In some embodiments, a biological sample may be blood and sometimes plasma or serum. As used herein, the term “blood” encompasses whole blood or any fractions of blood, such as serum and plasma as conventionally defined, for example. Blood plasma refers to the fraction of whole blood resulting from centrifugation of blood treated with anticoagulants. Blood serum refers to the watery portion of fluid remaining after a blood sample has coagulated. Fluid or tissue samples often are collected in accordance with standard protocols hospitals or clinics generally follow. For blood, an appropriate amount of peripheral blood (e.g., between 3-40 milliliters) often is collected and can be stored according to standard procedures prior to further preparation. A fluid or tissue sample from which nucleic acid is extracted may be acellular. In some embodiments, a fluid or tissue sample may contain cellular elements or cellular remnants. In some embodiments fetal cells or cancer cells may be included in the sample.

A sample often is heterogeneous, by which is meant that more than one type of nucleic acid species is present in the sample. For example, heterogeneous nucleic acid can include, but is not limited to, (i) fetally derived and maternally derived nucleic acid, (ii) cancer and non-cancer nucleic acid, (iii) pathogen and host nucleic acid, and more generally, (iv) mutated and wild-type nucleic acid. A sample may be heterogeneous because more than one cell type is present, such as a fetal cell and a maternal cell, a cancer and non-cancer cell, or a pathogenic and host cell. In some embodiments, a minority nucleic acid species and a majority nucleic acid species is present. Heterogeneous nucleic acid is addressed in greater detail herein.

For prenatal applications of technology described herein, fluid or tissue sample may be collected from a female at a gestational age suitable for testing, or from a female who is being tested for possible pregnancy. Suitable gestational age may vary depending on the prenatal test being performed. In certain embodiments, a pregnant female subject sometimes is in the first trimester of pregnancy, at times in the second trimester of pregnancy, or sometimes in the third trimester of pregnancy. In certain embodiments, a fluid or tissue is collected from a pregnant female between about 1 to about 45 weeks of fetal gestation (e.g., at 1-4, 4-8, 8-12, 12-16, 16-20, 20-24, 24-28, 28-32, 32-36, 36-40 or 40-44 weeks of fetal gestation), and sometimes between about 5 to about 28 weeks of fetal gestation (e.g., at 6, 7, 8, 9, 10, 11, 12, 13, 14, 15, 16, 17, 18, 19, 20, 21, 22, 23, 24, 25, 26 or 27 weeks of fetal gestation).

### Nucleic Acid Isolation and Processing

Nucleic acid may be derived from one or more sources (e.g., cells, soil, etc.) by methods known in the art. Cell lysis procedures and reagents are known in the art and may generally be performed by chemical, physical, or electrolytic lysis methods. For example, chemical methods generally employ lysing agents to disrupt cells and extract the nucleic acids from the cells, followed by treatment with chaotropic salts. Physical methods such as freeze/thaw followed by grinding, the use of cell presses and the like also are useful. High salt lysis procedures also are commonly used. For example, an alkaline lysis procedure may be utilized. The latter procedure traditionally incorporates the use of phenol-chloroform solutions, and an alternative phenol-chloroform-free procedure involving three solutions can be utilized. In the latter procedures, one solution can contain 15 mM Tris, pH 8.0; 10 mM EDTA and 100 ug/ml Rnase A; a second solution can contain 0.2N NaOH and 1% SDS; and a third solution can contain 3M KOAc, pH 5.5. These procedures can be found in Current Protocols in Molecular Biology, John Wiley & Sons, N.Y., 6.3.1-6.3.6 (1989), incorporated herein in its entirety.

The terms “nucleic acid” and “nucleic acid molecule” are used interchangeably. The terms refer to nucleic acids of any composition form, such as deoxyribonucleic acid (DNA, e.g., complementary DNA (cDNA), genomic DNA (gDNA) and the like), ribonucleic acid (RNA, e.g., message RNA (mRNA), short inhibitory RNA (siRNA), ribosomal RNA (rRNA), transfer RNA (tRNA), microRNA, RNA highly expressed by the fetus or placenta, and the like), and/or DNA or RNA analogs (e.g., containing base analogs, sugar analogs and/or a non-native backbone and the like), RNA/DNA hybrids and polyamide nucleic acids (PNAs), all of which can be in single- or double-stranded form. Unless otherwise limited, a nucleic acid can comprise known analogs of natural nucleotides, some of which can function in a similar manner as naturally occurring nucleotides. A nucleic acid can be in any form useful for conducting processes herein (e.g., linear, circular, supercoiled, single-stranded, double-stranded and the like). A nucleic acid may be, or may be from, a plasmid, phage, autonomously replicating sequence (ARS), centromere, artificial chromosome, chromosome, or other nucleic acid able to replicate or be replicated in vitro or in a host cell, a cell, a cell nucleus or cytoplasm of a cell in certain embodiments. A nucleic acid in some embodiments can be from a single chromosome (e.g., a nucleic acid sample may be from one chromosome of a sample obtained from a diploid organism). Nucleic acids also include derivatives, variants and analogs of RNA or DNA synthesized, replicated or amplified from single-stranded (“sense” or “antisense”, “plus” strand or “minus” strand, “forward” reading frame or “reverse” reading frame) and double-stranded polynucleotides. Deoxyribonucleotides include deoxyadenosine, deoxycytidine, deoxyguanosine and deoxythymidine. For RNA, the base cytosine is replaced with uracil and the sugar 2′ position includes a hydroxyl moiety. A nucleic acid may be prepared using a nucleic acid obtained from a subject as a template.

Nucleic acid may be isolated at a different time point as compared to another nucleic acid, where each of the samples is from the same or a different source. A nucleic acid may be from a nucleic acid library, such as a cDNA or RNA library, for example. A nucleic acid may be a result of nucleic acid purification or isolation and/or amplification of nucleic acid molecules from the sample. Nucleic acid provided for processes described herein may contain nucleic acid from one sample or from two or more samples (e.g., from 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, or 20 or more samples).

Nucleic acid can include extracellular nucleic acid in certain embodiments. The term “extracellular nucleic acid” as used herein refers to nucleic acid isolated from a source having substantially no cells and also is referred to as “cell-free” nucleic acid and/or “cell-free circulating” nucleic acid. Extracellular nucleic acid often includes no detectable cells and may contain cellular elements or cellular remnants. Non-limiting examples of acellular sources for extracellular nucleic acid are blood plasma, blood serum and urine. As used herein, the term “obtain cell-free circulating sample nucleic acid” includes obtaining a sample directly (e.g., collecting a sample) or obtaining a sample from another who has collected a sample. Without being limited by theory, extracellular nucleic acid may be a product of cell apoptosis and cell breakdown, which provides basis for extracellular nucleic acid often having a series of lengths across a spectrum (e.g., a “ladder”).

Extracellular nucleic acid can include different nucleic acid species, and therefore is referred to herein as “heterogeneous” in certain embodiments. For example, blood serum or plasma from a person having cancer can include nucleic acid from cancer cells and nucleic acid from non-cancer cells. In another example, blood serum or plasma from a pregnant female can include maternal nucleic acid and fetal nucleic acid. In some instances, fetal nucleic acid sometimes is about 5% to about 40% of the overall nucleic acid (e.g., about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38 or 39% of the total nucleic acid is fetal nucleic acid). In some embodiments, the majority of fetal nucleic acid in nucleic acid is of a length of about 500 base pairs or less (e.g., about 80, 85, 90, 91, 92, 93, 94, 95, 96, 97, 98, 99 or 100% of fetal nucleic acid is of a length of about 500 base pairs or less). In some embodiments, the majority of fetal nucleic acid in nucleic acid is of a length of about 250 base pairs or less (e.g., about 80, 85, 90, 91, 92, 93, 94, 95, 96, 97, 98, 99 or 100% of fetal nucleic acid is of a length of about 250 base pairs or less). In some embodiments, the majority of fetal nucleic acid in nucleic acid is of a length of about 200 base pairs or less (e.g., about 80, 85, 90, 91, 92, 93, 94, 95, 96, 97, 98, 99 or 100% of fetal nucleic acid is of a length of about 200 base pairs or less). In some embodiments, the majority of fetal nucleic acid in nucleic acid is of a length of about 150 base pairs or less (e.g., about 80, 85, 90, 91, 92, 93, 94, 95, 96, 97, 98, 99 or 100% of fetal nucleic acid is of a length of about 150 base pairs or less). In some embodiments, the majority of fetal nucleic acid in nucleic acid is of a length of about 100 base pairs or less (e.g., about 80, 85, 90, 91, 92, 93, 94, 95, 96, 97, 98, 99 or 100% of fetal nucleic acid is of a length of about 100 base pairs or less).

Nucleic acid may be provided for conducting methods described herein without processing of the sample(s) containing the nucleic acid, in certain embodiments. In some embodiments, nucleic acid is provided for conducting methods described herein after processing of the sample(s) containing the nucleic acid. For example, a nucleic acid may be extracted, isolated, purified or amplified from the sample(s). The term “isolated” as used herein refers to nucleic acid removed from its original environment (e.g., the natural environment if it is naturally occurring, or a host cell if expressed exogenously), and thus is altered by human intervention (e.g., “by the hand of man”) from its original environment. An isolated nucleic acid is provided with fewer non-nucleic acid components (e.g., protein, lipid) than the amount of components present in a source sample. A composition comprising isolated nucleic acid can be about 90%, 91%, 92%, 93%, 94%, 95%, 96%, 97%, 98%, 99% or greater than 99% free of non-nucleic acid components. The term “purified” as used herein refers to nucleic acid provided that contains fewer nucleic acid species than in the sample source from which the nucleic acid is derived. A composition comprising nucleic acid may be about 90%, 91%, 92%, 93%, 94%, 95%, 96%, 97%, 98%, 99% or greater than 99% free of other nucleic acid species. The term “amplified” as used herein refers to subjecting nucleic acid of a sample to a process that linearly or exponentially generates amplicon nucleic acids having the same or substantially the same nucleotide sequence as the nucleotide sequence of the nucleic acid in the sample, or portion thereof.

Nucleic acid also may be processed by subjecting nucleic acid to a method that generates nucleic acid fragments, in certain embodiments, before providing nucleic acid for a process described herein. In some embodiments, nucleic acid subjected to fragmentation or cleavage may have a nominal, average or mean length of about 5 to about 10,000 base pairs, about 100 to about 1,000 base pairs, about 100 to about 500 base pairs, or about 10, 15, 20, 25, 30, 35, 40, 45, 50, 55, 60, 65, 70, 75, 80, 85, 90, 95, 100, 200, 300, 400, 500, 600, 700, 800, 900, 1000, 2000, 3000, 4000, 5000, 6000, 7000, 8000 or 9000 base pairs. Fragments can be generated by any suitable method known in the art, and the average, mean or nominal length of nucleic acid fragments can be controlled by selecting an appropriate fragment-generating procedure. In certain embodiments, nucleic acid of a relatively shorter length can be utilized to analyze sequences that contain little sequence variation and/or contain relatively large amounts of known nucleotide sequence information. In some embodiments, nucleic acid of a relatively longer length can be utilized to analyze sequences that contain greater sequence variation and/or contain relatively small amounts of nucleotide sequence information.

Nucleic acid fragments may contain overlapping nucleotide sequences, and such overlapping sequences can facilitate construction of a nucleotide sequence of the non-fragmented counterpart nucleic acid, or a portion thereof. For example, one fragment may have subsequences x and y and another fragment may have subsequences y and z, where x, y and z are nucleotide sequences that can be 5 nucleotides in length or greater. Overlap sequence y can be utilized to facilitate construction of the x-y-z nucleotide sequence in nucleic acid from a sample in certain embodiments. Nucleic acid may be partially fragmented (e.g., from an incomplete or terminated specific cleavage reaction) or fully fragmented in certain embodiments.

Nucleic acid can be fragmented by various methods known in the art, which include without limitation, physical, chemical and enzymatic processes. Non-limiting examples of such processes are described in U.S. Patent Application Publication No. 2005/0112590 (published on May 26, 2005, entitled “Fragmentation-based methods and systems for sequence variation detection and discovery,” naming Van Den Boom et al.). Certain processes can be selected to generate non-specifically cleaved fragments or specifically cleaved fragments. Non-limiting examples of processes that can generate non-specifically cleaved fragment nucleic acid include, without limitation, contacting nucleic acid with apparatus that expose nucleic acid to shearing force (e.g., passing nucleic acid through a syringe needle; use of a French press); exposing nucleic acid to irradiation (e.g., gamma, x-ray, UV irradiation; fragment sizes can be controlled by irradiation intensity); boiling nucleic acid in water (e.g., yields about 500 base pair fragments) and exposing nucleic acid to an acid and base hydrolysis process.

As used herein, “fragmentation” or “cleavage” refers to a procedure or conditions in which a nucleic acid molecule, such as a nucleic acid template gene molecule or amplified product thereof, may be severed into two or more smaller nucleic acid molecules. Such fragmentation or cleavage can be sequence specific, base specific, or nonspecific, and can be accomplished by any of a variety of methods, reagents or conditions, including, for example, chemical, enzymatic, physical fragmentation.

As used herein, “fragments”, “cleavage products”, “cleaved products” or grammatical variants thereof, refers to nucleic acid molecules resultant from a fragmentation or cleavage of a nucleic acid template gene molecule or amplified product thereof. While such fragments or cleaved products can refer to all nucleic acid molecules resultant from a cleavage reaction, typically such fragments or cleaved products refer only to nucleic acid molecules resultant from a fragmentation or cleavage of a nucleic acid template gene molecule or the portion of an amplified product thereof containing the corresponding nucleotide sequence of a nucleic acid template gene molecule. For example, an amplified product can contain one or more nucleotides more than the amplified nucleotide region of a nucleic acid template sequence (e.g., a primer can contain “extra” nucleotides such as a transcriptional initiation sequence, in addition to nucleotides complementary to a nucleic acid template gene molecule, resulting in an amplified product containing “extra” nucleotides or nucleotides not corresponding to the amplified nucleotide region of the nucleic acid template gene molecule). Accordingly, fragments can include fragments arising from portions of amplified nucleic acid molecules containing, at least in part, nucleotide sequence information from or based on the representative nucleic acid template molecule.

As used herein, the term “complementary cleavage reactions” refers to cleavage reactions that are carried out on the same nucleic acid using different cleavage reagents or by altering the cleavage specificity of the same cleavage reagent such that alternate cleavage patterns of the same target or reference nucleic acid or protein are generated. In certain embodiments, nucleic acid may be treated with one or more specific cleavage agents (e.g., 1, 2, 3, 4, 5, 6, 7, 8, 9, 10 or more specific cleavage agents) in one or more reaction vessels (e.g., nucleic acid is treated with each specific cleavage agent in a separate vessel).

Nucleic acid may be specifically cleaved by contacting the nucleic acid with one or more specific cleavage agents. The term “specific cleavage agent” as used herein refers to an agent, sometimes a chemical or an enzyme that can cleave a nucleic acid at one or more specific sites. Specific cleavage agents often cleave specifically according to a particular nucleotide sequence at a particular site.

Examples of enzymatic specific cleavage agents include without limitation endonucleases (e.g., DNase (e.g., DNase I, II); RNase (e.g., RNase E, F, H, P); Cleavase™ enzyme; Taq DNA polymerase; E. coli DNA polymerase I and eukaryotic structure-specific endonucleases; murine FEN-1 endonucleases; type I, II or III restriction endonucleases such as Acc I, Afl III, Alu I, Alw44 I, Apa I, Asn I, Ava I, Ava II, BamH I, Ban II, Bcl I, Bgl I. Bgl II, Bln I, Bsm I, BssH II, BstE II, Cfo I, Cla I, Dde I, Dpn I, Dra I, EcIX I, EcoR I, EcoR I, EcoR II, EcoR V, Hae II, Hae II, Hind II, Hind III, Hpa I, Hpa II, Kpn I, Ksp I, Mlu I, MIuN I, Msp I, Nci I, Nco I, Nde I, Nde II, Nhe I, Not I, Nru I, Nsi I, Pst I, Pvu I, Pvu II, Rsa I, Sac I, Sal I, Sau3A I, Sca I, ScrF I, Sfi I, Sma I, Spe I, Sph I, Ssp I, Stu I, Sty I, Swa I, Taq I, Xba I, Xho I.); glycosylases (e.g., uracil-DNA glycosylase (UDG), 3-methyladenine DNA glycosylase, 3-methyladenine DNA glycosylase II, pyrimidine hydrate-DNA glycosylase, FaPy-DNA glycosylase, thymine mismatch-DNA glycosylase, hypoxanthine-DNA glycosylase, 5-Hydroxymethyluracil DNA glycosylase (HmUDG), 5-Hydroxymethylcytosine DNA glycosylase, or 1,N6-etheno-adenine DNA glycosylase); exonucleases (e.g., exonuclease III); ribozymes, and DNAzymes. Nucleic acid may be treated with a chemical agent, and the modified nucleic acid may be cleaved. In non-limiting examples, nucleic acid may be treated with (i) alkylating agents such as methylnitrosourea that generate several alkylated bases, including N3-methyladenine and N3-methylguanine, which are recognized and cleaved by alkyl purine DNA-glycosylase; (ii) sodium bisulfite, which causes deamination of cytosine residues in DNA to form uracil residues that can be cleaved by uracil N-glycosylase; and (iii) a chemical agent that converts guanine to its oxidized form, 8-hydroxyguanine, which can be cleaved by formamidopyrimidine DNA N-glycosylase. Examples of chemical cleavage processes include without limitation alkylation, (e.g., alkylation of phosphorothioate-modified nucleic acid); cleavage of acid lability of P3′-N5′-phosphoroamidate-containing nucleic acid; and osmium tetroxide and piperidine treatment of nucleic acid.

Nucleic acid also may be exposed to a process that modifies certain nucleotides in the nucleic acid before providing nucleic acid for a method described herein. A process that selectively modifies nucleic acid based upon the methylation state of nucleotides therein can be applied to nucleic acid, for example. In addition, conditions such as high temperature, ultraviolet radiation, x-radiation, can induce changes in the sequence of a nucleic acid molecule. Nucleic acid may be provided in any form useful for conducting a sequence analysis or manufacture process described herein, such as solid or liquid form, for example. In certain embodiments, nucleic acid may be provided in a liquid form optionally comprising one or more other components, including without limitation one or more buffers or salts.

Nucleic acid may be single or double stranded. Single stranded DNA, for example, can be generated by denaturing double stranded DNA by heating or by treatment with alkali, for example.

In some embodiments, nucleic acid is in a D-loop structure, formed by strand invasion of a duplex DNA molecule by an oligonucleotide or a DNA-like molecule such as peptide nucleic acid (PNA). D loop formation can be facilitated by addition of E. Coli RecA protein and/or by alteration of salt concentration, for example, using methods known in the art.

### Genomic Targets

In some embodiments, target nucleic acids, also referred to herein as target fragments, include polynucleotide fragments from a particular genomic region or plurality of genomic regions (e.g., single chromosome, set of chromosomes, and/or certain chromosome regions). In some embodiments, such genomic regions can be associated with fetal genetic abnormalities (e.g., aneuploidy) as well as other genetic variations including, but not limited to, mutations (e.g., point mutations), insertions, additions, deletions, translocations, trinucleotide repeat disorders, and/or single nucleotide polymorphisms (SNPs). In some embodiments, reference nucleic acids, also referred to herein as reference fragments, include polynucleotide fragments from a particular genomic region or plurality of genomic regions not associated with fetal genetic abnormalities. In some embodiments, target and/or reference nucleic acids (i.e., target fragments and/or reference fragments) comprise nucleotide sequences that are substantially unique to the chromosome of interest or reference chromosome (e.g., identical nucleotide sequences or substantially similar nucleotide sequences are not found elsewhere in the genome).

In some embodiments, fragments from a plurality of genomic regions are assayed. In some embodiments, target fragments and reference fragments from a plurality of genomic regions are assayed. In some embodiments, fragments from a plurality of genomic regions are assayed to determine the presence, absence, amount (e.g., relative amount) or ratio of a chromosome of interest, for example. In some embodiments, a chromosome of interest is a chromosome suspected of being aneuploid and may be referred to herein as a “test chromosome”. In some embodiments, fragments from a plurality of genomic regions is assayed for a presumed euploid chromosome. Such a chromosome may be referred to herein as a “reference chromosome”. In some embodiments, a plurality of test chromosomes is assayed. In some embodiments, test chromosomes are selected from among chromosome 13, chromosome 18 and chromosome 21. In some embodiments, reference chromosomes are selected from among chromosome 1, 2, 3, 4, 5, 6, 7, 8, 9, 10, 11, 12, 13, 14, 15, 16, 17, 18, 19, 20, 21, 22, X and Y, and sometimes, reference chromosomes are selected from autosomes (i.e., not X and Y). In some embodiments, chromosome 20 is selected as a reference chromosome. In some embodiments, chromosome 14 is selected as a reference chromosome. In some embodiments, chromosome 9 is selected as a reference chromosome. In some embodiments, a test chromosome and a reference chromosome are from the same individual. In some embodiments, a test chromosome and a reference chromosome are from different individuals.

In some embodiments, fragments from at least one genomic region are assayed for a test and/or reference chromosome. In some embodiments, fragments from at least 10 genomic regions (e.g., about 20, 30, 40, 50, 60, 70, 80 or 90 genomic regions) are assayed for a test chromosome and/or a reference chromosome. In some embodiments, fragments from at least 100 genomic regions (e.g., about 200, 300, 400, 500, 600, 700, 800 or 900 genomic regions) are assayed for a test chromosome and/or a reference chromosome. In some embodiments, fragments from at least 1,000 genomic regions (e.g., about 2000, 3000, 4000, 5000, 6000, 7000, 8000 or 9000 genomic regions) are assayed for a test chromosome and/or a reference chromosome. In some embodiments, fragments from at least 10,000 genomic regions (e.g., about 20,000, 30,000, 40,000, 50,000, 60,000, 70,000, 80,000 or 90,000 genomic regions) are assayed for a test chromosome and/or a reference chromosome. In some embodiments, fragments from at least 100,000 genomic regions (e.g., about 200,000, 300,000, 400,000, 500,000, 600,000, 700,000, 800,000 or 900,000 genomic regions) are assayed for a test chromosome and/or a reference chromosome.

### Determining Fetal Nucleic Acid Content

The amount of fetal nucleic acid (e.g., concentration, relative amount, absolute amount, copy number, and the like) in nucleic acid is determined in some embodiments. In some embodiments, the amount of fetal nucleic acid in a sample is referred to as “fetal fraction”. Fetal fraction can be determined, in some embodiments, using methods described herein for determining fragment length. Cell-free fetal nucleic acid fragments generally are shorter than maternally-derived nucleic acid fragments (see e.g., Chan et al. (2004) Clin. Chem. 50:88-92; Lo et al. (2010) Sci. Transl. Med. 2:61ra91). Thus, fetal fraction can be determined, in some embodiments, by counting fragments under a particular length threshold and comparing the counts to the amount of total nucleic acid in the sample. Methods for counting nucleic acid fragments of a particular length are described in further detail below.

In certain embodiments, the amount of fetal nucleic acid is determined according to markers specific to a male fetus (e.g., Y-chromosome STR markers (e.g., DYS 19, DYS 385, DYS 392 markers); RhD marker in RhD-negative females), allelic ratios of polymorphic sequences, or according to one or more markers specific to fetal nucleic acid and not maternal nucleic acid (e.g., differential epigenetic biomarkers (e.g., methylation; described in further detail below) between mother and fetus, or fetal RNA markers in maternal blood plasma (see e.g., Lo, 2005, Journal of Histochemistry and Cytochemistry 53 (3): 293-296)).

Determination of fetal nucleic acid content (e.g., fetal fraction) sometimes is performed using a fetal quantifier assay (FQA) as described, for example, in U.S. Patent Application Publication No. 2010/0105049, which is hereby incorporated by reference. This type of assay allows for the detection and quantification of fetal nucleic acid in a maternal sample based on the methylation status of the nucleic acid in the sample. In some embodiments, the amount of fetal nucleic acid from a maternal sample can be determined relative to the total amount of nucleic acid present, thereby providing the percentage of fetal nucleic acid in the sample. In some embodiments, the copy number of fetal nucleic acid can be determined in a maternal sample. In some embodiments, the amount of fetal nucleic acid can be determined in a sequence-specific (or locus-specific) manner and sometimes with sufficient sensitivity to allow for accurate chromosomal dosage analysis (for example, to detect the presence or absence of a fetal aneuploidy).

A fetal quantifier assay (FQA) can be performed in conjunction with any of the methods described herein. Such an assay can be performed by any method known in the art and/or described in U.S. Patent Application Publication No. 2010/0105049, such as, for example, by a method that can distinguish between maternal and fetal DNA based on differential methylation status, and quantify (i.e. determine the amount of) the fetal DNA. Methods for differentiating nucleic acid based on methylation status include, but are not limited to, methylation sensitive capture, for example, using a MBD2-Fc fragment in which the methyl binding domain of MBD2 is fused to the Fc fragment of an antibody (MBD-FC) (Gebhard et al. (2006) Cancer Res. 66(12):6118-28); methylation specific antibodies; bisulfite conversion methods, for example, MSP (methylation-sensitive PCR), COBRA, methylation-sensitive single nucleotide primer extension (Ms-SNuPE) or Sequenom MassCLEAVE™ technology; and the use of methylation sensitive restriction enzymes (e.g., digestion of maternal DNA in a maternal sample using one or more methylation sensitive restriction enzymes thereby enriching the fetal DNA). Methyl-sensitive enzymes also can be used to differentiate nucleic acid based on methylation status, which, for example, can preferentially or substantially cleave or digest at their DNA recognition sequence if the latter is non-methylated. Thus, an unmethylated DNA sample will be cut into smaller fragments than a methylated DNA sample and a hypermethylated DNA sample will not be cleaved. Except where explicitly stated, any method for differentiating nucleic acid based on methylation status can be used with the compositions and methods of the technology herein. The amount of fetal DNA can be determined, for example, by introducing one or more competitors at known concentrations during an amplification reaction. Determining the amount of fetal DNA also can be done, for example, by RT-PCR, primer extension, sequencing and/or counting. In certain instances, the amount of nucleic acid can be determined using BEAMing technology as described in U.S. Patent Application Publication No. 2007/0065823. In some embodiments, the restriction efficiency can be determined and the efficiency rate is used to further determine the amount of fetal DNA.

In some embodiments, a fetal quantifier assay (FQA) can be used to determine the concentration of fetal DNA in a maternal sample, for example, by the following method: a) determine the total amount of DNA present in a maternal sample; b) selectively digest the maternal DNA in a maternal sample using one or more methylation sensitive restriction enzymes thereby enriching the fetal DNA; c) determine the amount of fetal DNA from step b); and d) compare the amount of fetal DNA from step c) to the total amount of DNA from step a), thereby determining the concentration of fetal DNA in the maternal sample. In some embodiments, the absolute copy number of fetal nucleic acid in a maternal sample can be determined, for example, using mass spectrometry and/or a system that uses a competitive PCR approach for absolute copy number measurements. See for example, Ding and Cantor (2003) Proc Natl Acad Sci USA 100:3059-3064, and U.S. Patent Application Publication No. 2004/0081993, both of which are hereby incorporated by reference.

In some embodiments, fetal fraction can be determined based on allelic ratios of polymorphic sequences (e.g., single nucleotide polymorphisms (SNPs)), such as, for example, using a method described in U.S. Patent Application Publication No. 2011/0224087, which is hereby incorporated by reference. In such a method, nucleotide sequence reads are obtained for a maternal sample and fetal fraction is determined by comparing the total number of nucleotide sequence reads that map to a first allele and the total number of nucleotide sequence reads that map to a second allele at an informative polymorphic site (e.g., SNP) in a reference genome. In some embodiments, fetal alleles are identified, for example, by their relative minor contribution to the mixture of fetal and maternal nucleic acids in the sample when compared to the major contribution to the mixture by the maternal nucleic acids. Accordingly, the relative abundance of fetal nucleic acid in a maternal sample can be determined as a parameter of the total number of unique sequence reads mapped to a target nucleic acid sequence on a reference genome for each of the two alleles of a polymorphic site.

The amount of fetal nucleic acid in extracellular nucleic acid can be quantified and used in conjunction with the methods provided herein. Thus, in certain embodiments, methods of the technology described herein comprise an additional step of determining the amount of fetal nucleic acid. The amount of fetal nucleic acid can be determined in a nucleic acid sample from a subject before or after processing to prepare sample nucleic acid. In certain embodiments, the amount of fetal nucleic acid is determined in a sample after sample nucleic acid is processed and prepared, which amount is utilized for further assessment. In some embodiments, an outcome comprises factoring the fraction of fetal nucleic acid in the sample nucleic acid (e.g., adjusting counts, removing samples, making a call or not making a call).

The determination step can be performed before, during, at any one point in a method described herein, or after certain (e.g., aneuploidy detection) methods described herein. For example, to achieve an aneuploidy detection method with a given sensitivity or specificity, a fetal nucleic acid quantification method may be implemented prior to, during or after aneuploidy detection to identify those samples with greater than about 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25% or more fetal nucleic acid. In some embodiments, samples determined as having a certain threshold amount of fetal nucleic acid (e.g., about 15% or more fetal nucleic acid; about 4% or more fetal nucleic acid) are further analyzed for the presence or absence of aneuploidy or genetic variation. In certain embodiments, determinations of the presence or absence of aneuploidy are selected (e.g., selected and communicated to a patient) only for samples having a certain threshold amount of fetal nucleic acid (e.g., about 15% or more fetal nucleic acid; about 4% or more fetal nucleic acid).

### Enriching for a Subpopulation of Nucleic Acid

In some embodiments, nucleic acid (e.g., extracellular nucleic acid) is enriched or relatively enriched for a subpopulation or species of nucleic acid. Nucleic acid subpopulations can include, for example, fetal nucleic acid, maternal nucleic acid, nucleic acid comprising fragments of a particular length or range of lengths, or nucleic acid from a particular genome region (e.g., single chromosome, set of chromosomes, and/or certain chromosome regions). Such enriched samples can be used in conjunction with the methods provided herein. Thus, in certain embodiments, methods of the technology comprise an additional step of enriching for a subpopulation of nucleic acid in a sample, such as, for example, fetal nucleic acid. In some embodiments, a method for determining fetal fraction described above also can be used to enrich for fetal nucleic acid. In certain embodiments, maternal nucleic acid is selectively removed (partially, substantially, almost completely or completely) from the sample. In some embodiments, enriching for a particular low copy number species nucleic acid (e.g., fetal nucleic acid) may improve quantitative sensitivity. Methods for enriching a sample for a particular species of nucleic acid are described, for example, in U.S. Pat. No. 6,927,028, International Patent Application Publication No. WO2007/140417, International Patent Application Publication No. WO2007/147063, International Patent Application Publication No. WO2009/032779, International Patent Application Publication No. WO2009/032781, International Patent Application Publication No. WO2010/033639, International Patent Application Publication No. WO2011/034631, International Patent Application Publication No. WO2006/056480, and International Patent Application Publication No. WO2011/143659, all of which are incorporated by reference herein. Certain methods for enriching for a nucleic acid subpopulation (e.g., fetal nucleic acid) in a sample are described in detail below.

Some methods for enriching for a nucleic acid subpopulation (e.g., fetal nucleic acid) that can be used with the methods described herein include methods that exploit epigenetic differences between maternal and fetal nucleic acid. For example, fetal nucleic acid can be differentiated and separated from maternal nucleic acid based on methylation differences. Methylation-based fetal nucleic acid enrichment methods are described in U.S. Patent Application Publication No. 2010/0105049, which is incorporated by reference herein. Such methods sometimes involve binding a sample nucleic acid to a methylation-specific binding agent (methyl-CpG binding protein (MBD), methylation specific antibodies, and the like) and separating bound nucleic acid from unbound nucleic acid based on differential methylation status. Such methods also can include the use of methylation-sensitive restriction enzymes (as described above; e.g., HhaI and HpaII), which allow for the enrichment of fetal nucleic acid regions in a maternal sample by selectively digesting nucleic acid from the maternal sample with an enzyme that selectively and completely or substantially digests the maternal nucleic acid to enrich the sample for at least one fetal nucleic acid region.

Another method for enriching for a nucleic acid subpopulation (e.g., fetal nucleic acid) that can be used with the methods described herein is a restriction endonuclease enhanced polymorphic sequence approach, such as a method described in U.S. Patent Application Publication No. 2009/0317818, which is incorporated by reference herein. Such methods include cleavage of nucleic acid comprising a non-target allele with a restriction endonuclease that recognizes the nucleic acid comprising the non-target allele but not the target allele; and amplification of uncleaved nucleic acid but not cleaved nucleic acid, where the uncleaved, amplified nucleic acid represents enriched target nucleic acid (e.g., fetal nucleic acid) relative to non-target nucleic acid (e.g., maternal nucleic acid). In some embodiments, nucleic acid may be selected such that it comprises an allele having a polymorphic site that is susceptible to selective digestion by a cleavage agent, for example.

Some methods for enriching for a nucleic acid subpopulation (e.g., fetal nucleic acid) that can be used with the methods described herein include selective enzymatic degradation approaches. Such methods involve protecting target sequences from exonuclease digestion thereby facilitating the elimination in a sample of undesired sequences (e.g., maternal DNA). For example, in one approach, sample nucleic acid is denatured to generate single stranded nucleic acid, single stranded nucleic acid is contacted with at least one target-specific primer pair under suitable annealing conditions, annealed primers are extended by nucleotide polymerization generating double stranded target sequences, and digesting single stranded nucleic acid using a nuclease that digests single stranded (i.e. non-target) nucleic acid. In some embodiments, the method can be repeated for at least one additional cycle. In some embodiments, the same target-specific primer pair is used to prime each of the first and second cycles of extension, and in some embodiments, different target-specific primer pairs are used for the first and second cycles.

Some methods for enriching for a nucleic acid subpopulation (e.g., fetal nucleic acid) that can be used with the methods described herein include massively parallel signature sequencing (MPSS) approaches. MPSS typically is a solid phase method that uses adapter (i.e. tag) ligation, followed by adapter decoding, and reading of the nucleic acid sequence in small increments. Tagged PCR products are typically amplified such that each nucleic acid generates a PCR product with a unique tag. Tags are often used to attach the PCR products to microbeads. After several rounds of ligation-based sequence determination, for example, a sequence signature can be identified from each bead. Each signature sequence (MPSS tag) in a MPSS dataset is analyzed, compared with all other signatures, and all identical signatures are counted.

In some embodiments, certain MPSS-based enrichment methods can include amplification (e.g., PCR)-based approaches. In some embodiments, loci-specific amplification methods can be used (e.g., using loci-specific amplification primers). In some embodiments, a multiplex SNP allele PCR approach can be used. In some embodiments, a multiplex SNP allele PCR approach can be used in combination with uniplex sequencing. For example, such an approach can involve the use of multiplex PCR (e.g., MASSARRAY system) and incorporation of capture probe sequences into the amplicons followed by sequencing using, for example, the Illumina MPSS system. In some embodiments, a multiplex SNP allele PCR approach can be used in combination with a three-primer system and indexed sequencing. For example, such an approach can involve the use of multiplex PCR (e.g., MASSARRAY system) with primers having a first capture probe incorporated into certain loci-specific forward PCR primers and adapter sequences incorporated into loci-specific reverse PCR primers, to thereby generate amplicons, followed by a secondary PCR to incorporate reverse capture sequences and molecular index barcodes for sequencing using, for example, the Illumina MPSS system. In some embodiments, a multiplex SNP allele PCR approach can be used in combination with a four-primer system and indexed sequencing. For example, such an approach can involve the use of multiplex PCR (e.g., MASSARRAY system) with primers having adaptor sequences incorporated into both loci-specific forward and loci-specific reverse PCR primers, followed by a secondary PCR to incorporate both forward and reverse capture sequences and molecular index barcodes for sequencing using, for example, the Illumina MPSS system. In some embodiments, a microfluidics approach can be used. In some embodiments, an array-based microfluidics approach can be used. For example, such an approach can involve the use of a microfluidics array (e.g., Fluidigm) for amplification at low plex and incorporation of index and capture probes, followed by sequencing. In some embodiments, an emulsion microfluidics approach can be used, such as, for example, digital droplet PCR.

In some embodiments, universal amplification methods can be used (e.g., using universal or non-loci-specific amplification primers). In some embodiments, universal amplification methods can be used in combination with pull-down approaches. In some embodiments, the method can include biotinylated ultramer pull-down (e.g., biotinylated pull-down assays from Agilent or IDT) from a universally amplified sequencing library. For example, such an approach can involve preparation of a standard library, enrichment for selected regions by a pull-down assay, and a secondary universal amplification step. In some embodiments, pull-down approaches can be used in combination with ligation-based methods. In some embodiments, the method can include biotinylated ultramer pull down with sequence specific adapter ligation (e.g., HALOPLEX PCR, Halo Genomics). For example, such an approach can involve the use of selector probes to capture restriction enzyme-digested fragments, followed by ligation of captured products to an adaptor, and universal amplification followed by sequencing. In some embodiments, pull-down approaches can be used in combination with extension and ligation-based methods. In some embodiments, the method can include molecular inversion probe (MIP) extension and ligation. For example, such an approach can involve the use of molecular inversion probes in combination with sequence adapters followed by universal amplification and sequencing. In some embodiments, complementary DNA can be synthesized and sequenced without amplification.

In some embodiments, extension and ligation approaches can be performed without a pull-down component. In some embodiments, the method can include loci-specific forward and reverse primer hybridization, extension and ligation. Such methods can further include universal amplification or complementary DNA synthesis without amplification, followed by sequencing. Such methods can reduce or exclude background sequences during analysis, in some embodiments.

In some embodiments, pull-down approaches can be used with an optional amplification component or with no amplification component. In some embodiments, the method can include a modified pull-down assay and ligation with full incorporation of capture probes without universal amplification. For example, such an approach can involve the use of modified selector probes to capture restriction enzyme-digested fragments, followed by ligation of captured products to an adaptor, optional amplification, and sequencing. In some embodiments, the method can include a biotinylated pull-down assay with extension and ligation of adaptor sequence in combination with circular single stranded ligation. For example, such an approach can involve the use of selector probes to capture regions of interest (i.e. target sequences), extension of the probes, adaptor ligation, single stranded circular ligation, optional amplification, and sequencing. In some embodiments, the analysis of the sequencing result can separate target sequences form background.

In some embodiments, nucleic acid is enriched for certain target fragment species and/or reference fragment species. In some embodiments, nucleic acid is enriched for a specific nucleic acid fragment length or range of fragment lengths using one or more length-based separation methods described herein. In some embodiments, nucleic acid is enriched for fragments from a select genomic region (e.g., chromosome) using one or more sequence-based separation methods described herein. Such length-based and sequence-based separation methods are described in further detail below.

### Nucleic Acid Separation

In some embodiments, nucleic acid is enriched for certain target fragment species and/or reference fragment species using a nucleic acid separation method. In some embodiments, nucleic acid is enriched for a specific nucleic acid fragment length or range of fragment lengths using one or more length-based separation methods described herein. In some embodiments, nucleic acid is enriched for fragments from a select genomic region (e.g., chromosome) using one or more sequence-based separation methods described herein. In some embodiments, nucleic acid is enriched for a specific polynucleotide fragment length or range of fragment lengths and for fragments from a select genomic region (e.g., chromosome) using a combination of length-based and sequence-based separation methods. Such length-based and sequence-based separation methods are described in further detail below.

Sequence-Based Separation

In some embodiments, nucleic acid is enriched for fragments from a select genomic region (e.g., chromosome) using one or more sequence-based separation methods described herein. Sequence-based separation generally is based on nucleotide sequences present in the fragments of interest (e.g., target and/or reference fragments) and substantially not present in other fragments of the sample or present in an insubstantial amount of the other fragments (e.g., 5% or less). In some embodiments, sequence-based separation can generate separated target fragments and/or separated reference fragments. Separated target fragments and/or separated reference fragments typically are isolated away from the remaining fragments in the nucleic acid sample. In some embodiments, the separated target fragments and the separated reference fragments also are isolated away from each other (e.g., isolated in separate assay compartments). In some embodiments, the separated target fragments and the separated reference fragments are isolated together (e.g., isolated in the same assay compartment). In some embodiments, unbound fragments can be differentially removed or degraded or digested.

In some embodiments, a selective nucleic acid capture process is used to separate target and/or reference fragments away from the nucleic acid sample. Commercially available nucleic acid capture systems include, for example, Nimblegen sequence capture system (Roche NimbleGen, Madison, Wis.); Illumina BEADARRAY platform (Illumina, San Diego, Calif.); Affymetrix GENECHIP platform (Affymetrix, Santa Clara, Calif.); Agilent SureSelect Target Enrichment System (Agilent Technologies, Santa Clara, Calif.); and related platforms. Such methods typically involve hybridization of a capture oligonucleotide to a portion or all of the nucleotide sequence of a target or reference fragment and can include use of a solid phase (e.g., solid phase array) and/or a solution based platform. Capture oligonucleotides (sometimes referred to as “bait”) can be selected or designed such that they preferentially hybridize to nucleic acid fragments from selected genomic regions or loci (e.g., one of chromosomes 21, 18, 13, or X or a reference chromosome).

Capture oligonucleotides typically comprise a nucleotide sequence capable of hybridizing or annealing to a nucleic acid fragment of interest (e.g. target fragment, reference fragment) or a portion thereof. A capture oligonucleotide may be naturally occurring or synthetic and may be DNA or RNA based. Capture oligonucleotides can allow for specific separation of, for example, a target and/or reference fragment away from other fragments in a nucleic acid sample. The term “specific” or “specificity”, as used herein, refers to the binding or hybridization of one molecule to another molecule, such as an oligonucleotide for a target polynucleotide. “Specific” or “specificity” refers to the recognition, contact, and formation of a stable complex between two molecules, as compared to substantially less recognition, contact, or complex formation of either of those two molecules with other molecules. As used herein, the term “anneal” refers to the formation of a stable complex between two molecules. The terms “capture oligonucleotide”, “capture oligo”, “oligo”, or “oligonucleotide” may be used interchangeably throughout the document, when referring to capture oligonucleotides. In some embodiments, a probe described herein can be a capture oligonucleotide. The following features of oligonucleotides can be applied to primers and other oligonucleotides, such as probes provided herein.

A capture oligonucleotide can be designed and synthesized using a suitable process, and may be of any length suitable for hybridizing to a nucleotide sequence of interest and performing separation and/or analysis processes described herein. Oligonucleotides may be designed based upon a nucleotide sequence of interest (e.g., target fragment sequence, reference fragment sequence). An oligonucleotide, in some embodiments, may be about 10 to about 300 nucleotides, about 10 to about 100 nucleotides, about 10 to about 70 nucleotides, about 10 to about 50 nucleotides, about 15 to about 30 nucleotides, or about 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95 or 100 nucleotides in length. An oligonucleotide may be composed of naturally occurring and/or non-naturally occurring nucleotides (e.g., labeled nucleotides), or a mixture thereof. Oligonucleotides suitable for use with embodiments described herein, may be synthesized and labeled using known techniques. Oligonucleotides may be chemically synthesized according to the solid phase phosphoramidite triester method first described by Beaucage and Caruthers (1981) Tetrahedron Letts. 22:1859-1862, using an automated synthesizer, and/or as described in Needham-VanDevanter et al. (1984) Nucleic Acids Res. 12:6159-6168. Purification of oligonucleotides can be effected by native acrylamide gel electrophoresis or by anion-exchange high-performance liquid chromatography (HPLC), for example, as described in Pearson and Regnier (1983) J. Chrom. 255:137-149.

All or a portion of an oligonucleotide sequence (naturally occurring or synthetic) may be substantially complementary to a target and/or reference fragment sequence or portion thereof, in some embodiments. As referred to herein, “substantially complementary” with respect to sequences refers to nucleotide sequences that will hybridize with each other. The stringency of the hybridization conditions can be altered to tolerate varying amounts of sequence mismatch. Included are target/reference and oligonucleotide sequences that are 55% or more, 56% or more, 57% or more, 58% or more, 59% or more, 60% or more, 61% or more, 62% or more, 63% or more, 64% or more, 65% or more, 66% or more, 67% or more, 68% or more, 69% or more, 70% or more, 71% or more, 72% or more, 73% or more, 74% or more, 75% or more, 76% or more, 77% or more, 78% or more, 79% or more, 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more or 99% or more complementary to each other.

Oligonucleotides that are substantially complimentary to a nucleic acid sequence of interest (e.g., target fragment sequence, reference fragment sequence) or portion thereof are also substantially similar to the compliment of the target nucleic acid sequence or relevant portion thereof (e.g., substantially similar to the anti-sense strand of the nucleic acid). One test for determining whether two nucleotide sequences are substantially similar is to determine the percent of identical nucleotide sequences shared. As referred to herein, “substantially similar” with respect to sequences refers to nucleotide sequences that are 55% or more, 56% or more, 57% or more, 58% or more, 59% or more, 60% or more, 61% or more, 62% or more, 63% or more, 64% or more, 65% or more, 66% or more, 67% or more, 68% or more, 69% or more, 70% or more, 71% or more, 72% or more, 73% or more, 74% or more, 75% or more, 76% or more, 77% or more, 78% or more, 79% or more, 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more or 99% or more identical to each other.

Annealing conditions (e.g., hybridization conditions) can be determined and/or adjusted, depending on the characteristics of the oligonucleotides used in an assay. In some embodiments, oligonucleotide sequence and/or length may affect hybridization to a nucleic acid sequence of interest. Depending on the degree of mismatch between an oligonucleotide and nucleic acid of interest, low, medium or high stringency conditions may be used to effect the annealing. As used herein, the term “stringent conditions” refers to conditions for hybridization and washing. Methods for hybridization reaction temperature condition optimization are known in the art, and may be found in Current Protocols in Molecular Biology, John Wiley & Sons, N.Y., 6.3.1-6.3.6 (1989). Aqueous and non-aqueous methods are described in that reference and either can be used. Non-limiting examples of stringent hybridization conditions are hybridization in 6× sodium chloride/sodium citrate (SSC) at about 45° C., followed by one or more washes in 0.2×SSC, 0.1% SDS at 50° C. Another example of stringent hybridization conditions are hybridization in 6× sodium chloride/sodium citrate (SSC) at about 45° C., followed by one or more washes in 0.2×SSC, 0.1% SDS at 55° C. A further example of stringent hybridization conditions is hybridization in 6× sodium chloride/sodium citrate (SSC) at about 45° C., followed by one or more washes in 0.2×SSC, 0.1% SDS at 60° C. Often, stringent hybridization conditions are hybridization in 6× sodium chloride/sodium citrate (SSC) at about 45° C., followed by one or more washes in 0.2×SSC, 0.1% SDS at 65° C. More often, stringency conditions are 0.5M sodium phosphate, 7% SDS at 65° C., followed by one or more washes at 0.2×SSC, 1% SDS at 65° C. Stringent hybridization temperatures can also be altered (i.e. lowered) with the addition of certain organic solvents, formamide for example. Organic solvents, like formamide, reduce the thermal stability of double-stranded polynucleotides, so that hybridization can be performed at lower temperatures, while still maintaining stringent conditions and extending the useful life of nucleic acids that may be heat labile.

As used herein, the phrase “hybridizing” or grammatical variations thereof, refers to annealing a first nucleic acid molecule to a second nucleic acid molecule under low, medium or high stringency conditions, or under nucleic acid synthesis conditions. Hybridizing can include instances where a first nucleic acid molecule anneals to a second nucleic acid molecule, where the first and second nucleic acid molecules are complementary. As used herein, “specifically hybridizes” refers to preferential hybridization under nucleic acid synthesis conditions of an oligonucleotide to a nucleic acid molecule having a sequence complementary to the oligonucleotide compared to hybridization to a nucleic acid molecule not having a complementary sequence. For example, specific hybridization includes the hybridization of a capture oligonucleotide to a target fragment sequence that is complementary to the oligonucleotide.

In some embodiments, one or more capture oligonucleotides are associated with an affinity ligand such as a member of a binding pair (e.g., biotin) or antigen that can bind to a capture agent such as avidin, streptavidin, an antibody, or a receptor. For example, a capture oligonucleotide may be biotinylated such that it can be captured onto a streptavidin-coated bead.

In some embodiments, one or more capture oligonucleotides and/or capture agents are effectively linked to a solid support or substrate. A solid support or substrate can be any physically separable solid to which a capture oligonucleotide can be directly or indirectly attached including, but not limited to, surfaces provided by microarrays and wells, and particles such as beads (e.g., paramagnetic beads, magnetic beads, microbeads, nanobeads), microparticles, and nanoparticles. Solid supports also can include, for example, chips, columns, optical fibers, wipes, filters (e.g., flat surface filters), one or more capillaries, glass and modified or functionalized glass (e.g., controlled-pore glass (CPG)), quartz, mica, diazotized membranes (paper or nylon), polyformaldehyde, cellulose, cellulose acetate, paper, ceramics, metals, metalloids, semiconductive materials, quantum dots, coated beads or particles, other chromatographic materials, magnetic particles; plastics (including acrylics, polystyrene, copolymers of styrene or other materials, polybutylene, polyurethanes, TEFLON™, polyethylene, polypropylene, polyamide, polyester, polyvinylidenedifluoride (PVDF), and the like), polysaccharides, nylon or nitrocellulose, resins, silica or silica-based materials including silicon, silica gel, and modified silicon, Sephadex®, Sepharose®, carbon, metals (e.g., steel, gold, silver, aluminum, silicon and copper), inorganic glasses, conducting polymers (including polymers such as polypyrrole and polyindole); micro or nanostructured surfaces such as nucleic acid tiling arrays, nanotube, nanowire, or nanoparticulate decorated surfaces; or porous surfaces or gels such as methacrylates, acrylamides, sugar polymers, cellulose, silicates, or other fibrous or stranded polymers. In some embodiments, the solid support or substrate may be coated using passive or chemically-derivatized coatings with any number of materials, including polymers, such as dextrans, acrylamides, gelatins or agarose. Beads and/or particles may be free or in connection with one another (e.g., sintered). In some embodiments, the solid phase can be a collection of particles. In some embodiments, the particles can comprise silica, and the silica may comprise silica dioxide. In some embodiments the silica can be porous, and in certain embodiments the silica can be non-porous. In some embodiments, the particles further comprise an agent that confers a paramagnetic property to the particles. In certain embodiments, the agent comprises a metal, and in certain embodiments the agent is a metal oxide, (e.g., iron or iron oxides, where the iron oxide contains a mixture of Fe2+ and Fe3+). The oligonucleotides may be linked to the solid support by covalent bonds or by non-covalent interactions and may be linked to the solid support directly or indirectly (e.g., via an intermediary agent such as a spacer molecule or biotin). A probe may be linked to the solid support before, during or after nucleic acid capture.

Length-Based Separation

In some embodiments, nucleic acid is enriched for a particular nucleic acid fragment length, range of lengths, or lengths under or over a particular threshold or cutoff using one or more length-based separation methods. Nucleic acid fragment length typically refers to the number of nucleotides in the fragment. Nucleic acid fragment length also is referred to herein as nucleic acid fragment size. In some embodiments, a length-based separation method is performed without measuring lengths of individual fragments. In some embodiments, a length based separation method is performed in conjunction with a method for determining length of individual fragments. In some embodiments, length-based separation refers to a size fractionation procedure where all or part of the fractionated pool can be isolated (e.g., retained) and/or analyzed. Size fractionation procedures are known in the art (e.g., separation on an array, separation by a molecular sieve, separation by gel electrophoresis, separation by column chromatography (e.g., size-exclusion columns), and microfluidics-based approaches). In some embodiments, length-based separation approaches can include fragment circularization, chemical treatment (e.g., formaldehyde, polyethylene glycol (PEG)), mass spectrometry and/or size-specific nucleic acid amplification, for example.

In some embodiments, nucleic acid fragments of a certain length, range of lengths, or lengths under or over a particular threshold or cutoff are separated from the sample. In some embodiments, fragments having a length under a particular threshold or cutoff (e.g., 500 bp, 400 bp, 300 bp, 200 bp, 150 bp, 100 bp) are referred to as “short” fragments and fragments having a length over a particular threshold or cutoff (e.g., 500 bp, 400 bp, 300 bp, 200 bp, 150 bp, 100 bp) are referred to as “long” fragments. In some embodiments, fragments of a certain length, range of lengths, or lengths under or over a particular threshold or cutoff are retained for analysis while fragments of a different length or range of lengths, or lengths over or under the threshold or cutoff are not retained for analysis. In some embodiments, fragments that are less than about 500 bp are retained. In some embodiments, fragments that are less than about 400 bp are retained. In some embodiments, fragments that are less than about 300 bp are retained. In some embodiments, fragments that are less than about 200 bp are retained. In some embodiments, fragments that are less than about 150 bp are retained. For example, fragments that are less than about 190 bp, 180 bp, 170 bp, 160 bp, 150 bp, 140 bp, 130 bp, 120 bp, 110 bp or 100 bp are retained. In some embodiments, fragments that are about 100 bp to about 200 bp are retained. For example, fragments that are about 190 bp, 180 bp, 170 bp, 160 bp, 150 bp, 140 bp, 130 bp, 120 bp or 110 bp are retained. In some embodiments, fragments that are in the range of about 100 bp to about 200 bp are retained. For example, fragments that are in the range of about 110 bp to about 190 bp, 130 bp to about 180 bp, 140 bp to about 170 bp, 140 bp to about 150 bp, 150 bp to about 160 bp, or 145 bp to about 155 bp are retained. In some embodiments, fragments that are about 10 bp to about 30 bp shorter than other fragments of a certain length or range of lengths are retained. In some embodiments, fragments that are about 10 bp to about 20 bp shorter than other fragments of a certain length or range of lengths are retained. In some embodiments, fragments that are about 10 bp to about 15 bp shorter than other fragments of a certain length or range of lengths are retained.

Certain length-based separation methods that can be used with methods described herein employ a selective sequence tagging approach, for example. In such methods, a fragment size species (e.g., short fragments) nucleic acids are selectively tagged in a sample that includes long and short nucleic acids. Such methods typically involve performing a nucleic acid amplification reaction using a set of nested primers which include inner primers and outer primers. In some embodiments, one or both of the inner can be tagged to thereby introduce a tag onto the target amplification product. The outer primers generally do not anneal to the short fragments that carry the (inner) target sequence. The inner primers can anneal to the short fragments and generate an amplification product that carries a tag and the target sequence. Typically, tagging of the long fragments is inhibited through a combination of mechanisms which include, for example, blocked extension of the inner primers by the prior annealing and extension of the outer primers. Enrichment for tagged fragments can be accomplished by any of a variety of methods, including for example, exonuclease digestion of single stranded nucleic acid and amplification of the tagged fragments using amplification primers specific for at least one tag.

Another length-based separation method that can be used with methods described herein involves subjecting a nucleic acid sample to polyethylene glycol (PEG) precipitation. Examples of methods include those described in International Patent Application Publication Nos. WO2007/140417 and WO2010/115016. This method in general entails contacting a nucleic acid sample with PEG in the presence of one or more monovalent salts under conditions sufficient to substantially precipitate large nucleic acids without substantially precipitating small (e.g., less than 300 nucleotides) nucleic acids.

Another size-based enrichment method that can be used with methods described herein involves circularization by ligation, for example, using circligase. Short nucleic acid fragments typically can be circularized with higher efficiency than long fragments. Non-circularized sequences can be separated from circularized sequences, and the enriched short fragments can be used for further analysis.

### Determination of Fragment Length

In some embodiments, length is determined for one or more nucleic acid fragments. In some embodiments, length is determined for one or more target fragments, thereby identifying one or more target fragment size species. In some embodiments, length is determined for one or more target fragments and one or more reference fragments, thereby identifying one or more target fragment length species and one or more reference fragment length species. In some embodiments, fragment length is determined by measuring the length of a probe that hybridizes to the fragment, which is discussed in further detail below. Nucleic acid fragment or probe length can be determined using any method in the art suitable for determining nucleic acid fragment length, such as, for example, a mass sensitive process (e.g., mass spectrometry (e.g., matrix-assisted laser desorption ionization (MALDI) mass spectrometry and electrospray (ES) mass spectrometry), electrophoresis (e.g., capillary electrophoresis), microscopy (scanning tunneling microscopy, atomic force microscopy), and measuring length using a nanopore. In some embodiments, fragment or probe length can be determined without use of a separation method based on fragment charge. In some embodiments, fragment or probe length can be determined without use of an electrophoresis process. In some embodiments, fragment or probe length can be determined without use of a nucleotide sequencing process.

Probes

In some embodiments, fragment length is determined using one or more probes. In some embodiments, probes are designed such that they each hybridize to a nucleic acid of interest in a sample. For example, a probe may comprise a polynucleotide sequence that is complementary to a nucleic acid of interest. Probes may be any length suitable to hybridize (e.g., completely hybridize) to one or more nucleic acid fragments of interest. For example, probes may be of any length which spans or extends beyond the length of a nucleic acid fragment to which it hybridizes. Probes may be about 100 bp or more in length. For example, probes may be at least about 200, 300, 400, 500, 600, 700, 800, 900 or 1000 bp in length.

In some embodiments, probes may comprise a polynucleotide sequence that is complementary to a nucleic acid of interest and one or more polynucleotide sequences that are not complementary to a nucleic acid of interest (i.e., non-complementary sequences). Non-complementary sequences may reside, for example, at the 5′ and/or 3′ end of a probe. In some embodiments, non-complementary sequences may comprise nucleotide sequences that do not exist in the organism of interest and/or sequences that are not capable of hybridizing to any sequence in the human genome. For example, non-complementary sequences may be derived from any non-human genome known in the art, such as, for example, non-mammalian animal genomes, plant genomes, fungal genomes, bacterial genomes, or viral genomes. In some embodiments, a non-complementary sequence is from the PhiX 174 genome. In some embodiments, a non-complementary sequence may comprise modified or synthetic nucleotides that are not capable of hybridizing to a complementary nucleotide.

Probes may be designed and synthesized according to methods known in the art and described herein for oligonucleotides (e.g., capture oligonucleotides). Probes also may include any of the properties known in the art and described herein for oligonucleotides. Probes herein may be designed such that they comprise one or more modified nucleotide species (e.g., modified adenine (A), modified thymine (T), modified cytosine (C), modified guanine (G) and/or modified uracil (U)), described in further detail below. In some embodiments, probes comprise a mixture of modified and unmodified nucleotide species. In some embodiments, probes comprise a first set of nucleotide species and a second set of nucleotide species. In some embodiments, the modified nucleotide species of the first set are purines, derivatives thereof or combinations thereof and modified nucleotide species of the second set are pyrimidines, derivatives thereof or combinations thereof. Probes herein generally are designed such that they initially have longer lengths than the fragments to which they hybridize.

In some embodiments, nucleic acid fragments (e.g., target and/or reference fragments) are contacted with one or more probes under annealing conditions, thereby generating fragment-probe species such as, for example, target-probe species and reference-probe species. Probes and/or hybridization conditions (e.g., stringency) can be optimized to favor complete or substantially complete fragment binding (e.g., high stringency). Complete or substantially complete fragment-probe hybridizations generally include duplexes where the fragment does not comprise unhybridized portions and the probe may comprise unhybridized portions, as described in further detail below.

In some embodiments, the target fragments and/or reference fragments are separated from the nucleic acid sample using a sequence-based separation method (e.g., selective nucleic acid capture process), as described above, prior to a probe hybridization step. In some embodiments, the target fragments and/or reference fragments are not separated from the nucleic acid sample prior to a probe hybridization step. For example, a sample may be contacted directly with the probes described herein. Such probes may serve as capture oligonucleotides for nucleic acid fragments of interest (e.g., target fragments and reference fragments). In some embodiments, probes can be designed using criteria described herein for capture oligonucleotides (e.g., associated with a solid support and/or binding partner) such that they hybridize to certain fragments in a sample and provide a means for separating the captured fragments from the sample.

Modified Nucleotides

Provided herein are modified nucleotides. Also provided herein are compositions and probes comprising modified nucleotides. Native nucleotides (e.g., adenine (A), thymine (T), cytosine (C), guanine (G) and uracil (U)) each possess unique separation properties (i.e., they can be resolved or distinguished from one another using a mass-sensitive process). Nucleotides generally comprise a purine or pyrimidine base, a sugar (e.g. ribose or deoxyribose), and one, two or three phosphate groups. Native nucleotide structures are shown below.

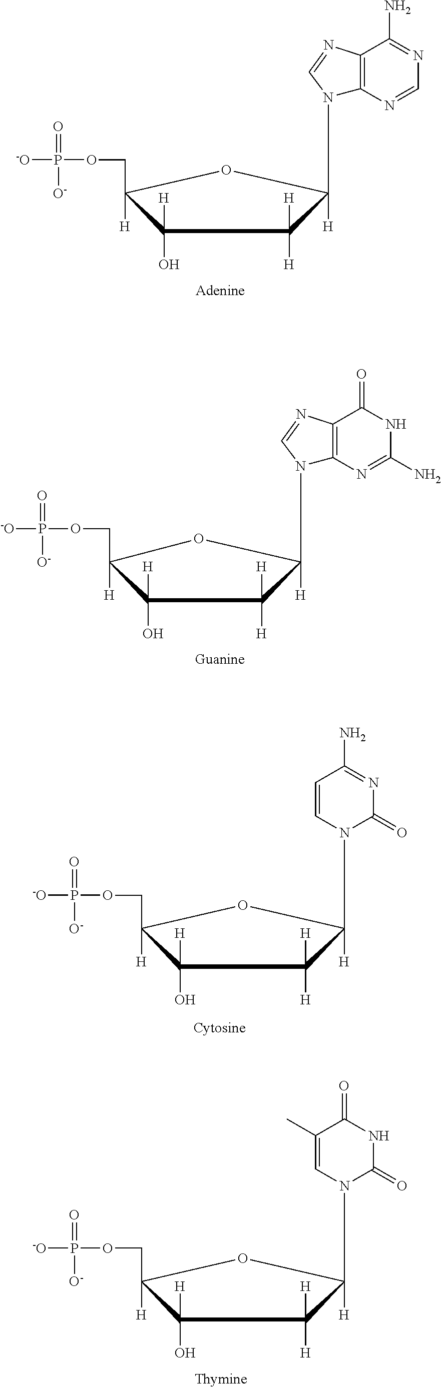

Native nucleotide base structures are shown below, which include purine and pyrimidine ring numbering:

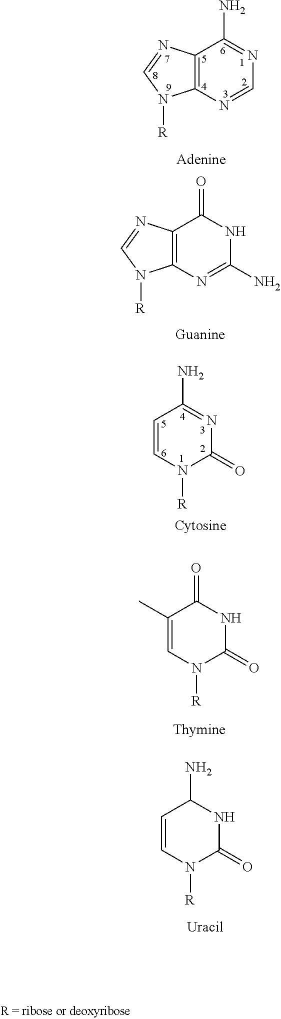

Certain nucleotide species herein are modified such that they have substantially identical separation properties when separated by a mass-sensitive process. Nucleotides having substantially identical separation properties generally cannot be resolved or distinguished from one another by a mass-sensitive process. For example, the difference between two nucleotide species having substantially identical separation properties cannot be detected by mass spectrometry. In some embodiments, a set of four nucleotide species having substantially identical separation properties is generated. In some embodiments, a first set of nucleotide species having substantially identical separation properties is generated and a second set of nucleotide species having substantially identical separation properties is generated, where the separation properties of the first set is different than the separation properties of the second set. In some embodiments, the nucleotide species of the first set are purines, derivatives thereof or combinations thereof and nucleotide species of the second set are pyrimidines, derivatives thereof or combinations thereof.

In some embodiments, polymers (e.g., polynucleotides) having an equal number of the modified nucleotides herein, regardless of nucleotide composition, have substantially identical separation properties when separated by a mass-sensitive process.

In some embodiments, the modified nucleotide species each are capable of hybridizing to (i.e., are complementary to) one of adenine (A), thymine (T), cytosine (C), guanine (G) and uracil (U). In some embodiments, the modified nucleotide species each are capable of hybridizing to (i.e., are complementary to) one of naturally occurring (e.g., native), modified or synthetic adenine (A), thymine (T), cytosine (C), guanine (G) and uracil (U). In some embodiments, the modified nucleotide species each are capable of hybridizing to (i.e., are complementary to) one of unmodified (e.g., not mass-modified) adenine (A), thymine (T), cytosine (C), guanine (G) and uracil (U). In some embodiments, the modified nucleotide species can each hybridize to their complementary nucleotide partner (e.g., Watson-Crick base pairing: A to T, G to C, A to U) with substantially similar binding specificity and/or strength as unmodified nucleotides. In some embodiments, hybridization conditions (e.g., stringency) can be adjusted according to methods described herein, for example, to facilitate hybridization of certain modified nucleotide species to their complementary partners (e.g., A-T, G-C, A-U).

Modified nucleotides herein can be in the form of modified nucleobases (e.g., purine or pyrimidine bases), modified nucleosides (e.g., nucleotide plus pentose (e.g., ribose, deoxyribose)), modified nucleoside monophosphates, modified nucleoside diphosphates, modified nucleoside triphosphates or derivatives or combinations thereof. In some embodiments, modified nucleotides are capable of forming phosphodiester bonds when polymerized. In some embodiments, modified nucleotides are polymerized using a template-dependent polymerase (e.g., DNA polymerase; RNA polymerase). For example, modified nucleotides can be substrates for a polymerase such that a template can be copied. In some embodiments, modified nucleotides are polymerized using a commercial synthesis method (e.g., automated synthesis method). Polymers comprising modified nucleotides can be designed using any molecular backbone structure suitable for such polymers which include, without limitation, nucleic acid backbones (e.g., phosphate-sugar backbones), fatty acid backbones, peptide backbones, peptide nucleic acid (PNA) backbones, and the like.

Native nucleotides (e.g., adenine (A), thymine (T), cytosine (C), guanine (G) and uracil (U)) each possess unique separation properties, in part, due to their differences in molar mass. Generally, differences in nucleotide molar mass are attributed to mass differences between the base species.

For example, the molar mass of A (C5H5N5) is 135.13 g/mol; the molar mass of T (C5H6N2O2) is 126.11 g/mol; the molar mass of C (C4H5N3O) is 111.10 g/mol; the molar mass of G (C5H5N5O) is 151.13; and the molar mass of U (C4H4N2O2) is 112.09 g/mol. In some embodiments, the modified nucleotides herein are mass-modified. The term “mass-modified nucleotide” refers to a nucleotide having a mass that differs from the naturally-occurring or native nucleotide. Mass-modified nucleotides may include a modified purine or pyrimidine base; a modified sugar; one, two, or three modified phosphate groups; or combinations thereof, where the modified base, sugar or phosphate(s) include one or more mass-modifying substituents (e.g., mass-modifiers). In some embodiments, a nucleotide comprises one or more mass-modifiers. In some embodiments, a purine or pyrimidine base comprises one or more mass-modifiers. In some embodiments, a sugar (e.g., ribose or deoxyribose) comprises one or more mass-modifiers. In some embodiments, a phosphate group (e.g., alpha phosphate) comprises one or more mass-modifiers. In some embodiments, one, two, three, four or more nucleotide species are mass-modified. In some embodiments, the modified nucleotides herein are mass-modified such that two or more nucleotide species have substantially identical masses. In some embodiments, the modified nucleotides herein are mass-modified such that three or more nucleotide species have substantially identical masses. In some embodiments, the modified nucleotides herein are mass-modified such that four or more nucleotide species have substantially identical masses. As used herein, substantially identical mass may include molar masses with a difference of less than about 1 atomic mass unit (AMU), less than about 0.1 atomic mass unit (AMU), less than about 0.01 atomic mass unit (AMU), and/or less than about 0.001 atomic mass unit (AMU).

A mass-modifying constituent can be any element or moiety that, when added to the nucleotide or substituted with an existing element or moiety, changes (e.g., increases or decreases) the molar mass of the nucleotide. In some embodiments, mass modifications to the nucleotides herein do not significantly interfere with Watson-Crick-specific base-pairing, and in certain embodiments, mass modifications do not interfere with formation and/or cleavage of a phosphodiester bond between nucleotides. A mass-modifying constituent can be present on, for example, the purine or pyrimidine base, the sugar, the phosphodiester linkage (e.g., alpha-thio-dNTPs), and/or any other location connected to or associated with the nucleotide. For example, a mass modifier can be located at any position in a purine or pyrimidine base. In some embodiments, a mass modifier can be located at C-8 in purine nucleotides or C-8 and/or C-7 in C7-deazapurine nucleotides, and at C-5 in uracil and cytosine and at the C-5 methyl group at thymine residues. Mass modification can also occur at the sugar moiety, such as at position C-2′.

Mass modifiers may include, for example, isotopes such as stable isotopes for oxygen (¹⁷O, ¹⁸O), nitrogen (e.g., nitrogen-15), carbon (e.g., carbon-13) and hydrogen (e.g., deuterium). In some embodiments, isotopically pure materials are used for synthesizing mass-modified nucleotides. For example, nucleotides comprising carbon-12 can be synthesized in the presence of carbon-13-free carbon-12. In some embodiments, elemental constituents of the nucleotide are replaced with isotopic variants. For example, a hydrogen atom having an atomic mass of about 1 AMU can be substituted with a deuterium atom (a hydrogen isotope) having an atomic mass of about 2 AMU, resulting in a net gain of about 1 AMU for the modified nucleotide. Such a modification in cytosine, for example, increases the molar mass of cytosine from about 111 to about 112, which is substantially identical to the molar mass of uracil. In some embodiments, a hydrogen to deuterium substitution is made at a non-exchangeable position in a nucleotide (e.g., hydrogen attached to carbon). In some embodiments, a hydrogen to deuterium substitution is made at an exchangeable position in a nucleotide (e.g., hydrogen attached to oxygen; hydrogen attached to nitrogen). In some embodiments, deuterium substitutions are made in the presence of deuterated water (i.e., D2O).

Mass modifiers also may include, for example, substituted elements (e.g., sulfur (e.g., isotopically pure sulfur) for oxygen, selenium (e.g., isotopically pure selenium) for oxygen, selenium for carbon, bromine or iodine (or other halogen) for hydrogen); mass tags (e.g., fluorescent mass tag or other chemical label); boron groups (e.g., boron-modified nucleotides); hydrocarbon groups (e.g., methyl group, ethyl group, propyl group, and the like) and other functional groups including, but not limited to, alkyl, alkenyl, alkynyl phenyl, and benzyl groups; haloalkane, fluoroalkane, chloroalkane, bromoalkane and iodoalkane groups; hydroxyl, carbonyl, aldehyde, haloformyl, carbonate ester, carboxylate, carboxyl, ester, hydroperoxy, peroxy, ether, hemiacetal, hemiketal, acetal, ketal, orthoester and orthocarbonate ester groups; amide, amine, imine, imide, azide, azo compound, cyanate, nitrate, nitrile, nitrite, nitro, nitroso, and pyridyl groups; thiol, sulfide, thioether, disulfide, sulfoxide, sulfone, sulfinic acid, sulfonic acid, thiocyanate, thione, and thial groups; phosphine, phosphane, phosphonic acid, phosphate and phosphodiester groups; boronic acid, boronic ester, borinic acid and borinic ester groups. In some embodiments, a hydrocarbon group or other functional group may comprise an isotope such as an isotope described above. For example, a methyl group mass-modifier may comprise carbon-13, and/or one, two, or three deuterium atoms.

In some embodiments, the amino group (—NH2) of one or more of adenine, guanine or cytosine bases can be modified by acylation. The amino acyl modification can be, for example, an acetyl, benzoyl, isobutyryl or anisoyl group. Benzoylchloride, in some embodiments, can acylate the amino group adenine, for example. In some embodiments, the sugar moiety can be the target of the mass-modification. For example, the sugar moieties can be acylated, tritylated, monomethoxytritylated, and the like. Other mass modifications to nucleotides are described, for example, in U.S. Pat. Nos. 5,547,835 and 6,140,053, which are incorporated by reference in their entirety. Methods for synthesizing modified nucleotides are known in the art (see e.g., Tolbert and Williamson (1996) J. Am. Chem. Soc. 118, 7929-7940; Batey et al. (1992) Nucl. Acids Res. 20, 4515-4523; Batey et al. (1996) Nucl. Acids Res. 24, 4836-4837; Tolbert and Williamson (1997) J. Am. Chem. Soc. 119, 12100-12108; Dayie et al. (1998) J. Mag. Reson. 130, 97-101 (1998); Mao and Williamson (1999) Nucl. Acids Res. 27, 4059-4070; Tang et al. (2002) Analytical Chemistry 74, 226-331; Scott et al. (2004) J. Am. Chem. Soc. 26, 11776-11777; Hennig et al (2007) J. Am. Chem. Soc. 129, 14911-14921).

Trimmed Probes

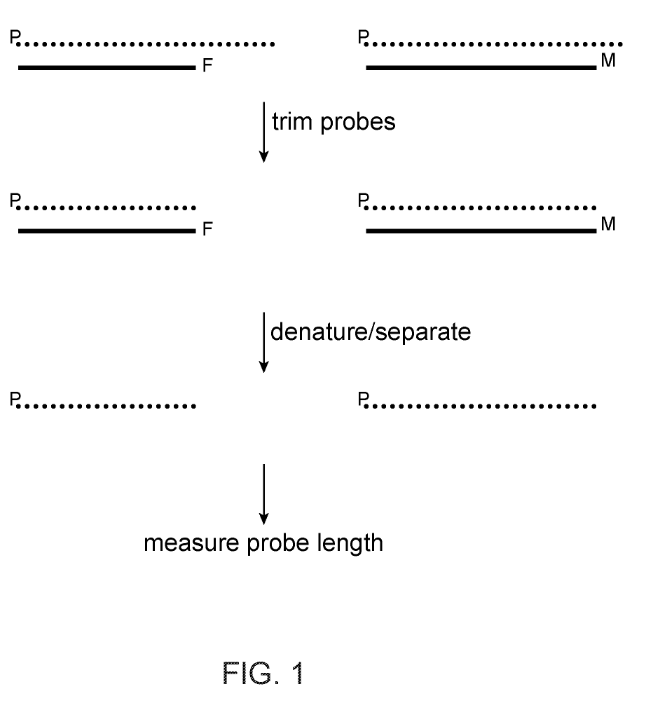

In some embodiments, a fragment-probe species (e.g., target-probe species and/or reference-probe species) may comprise one or more unhybridized probe portions (i.e., single stranded probe portions; e.g., FIG. 1), e.g., when the probe length is longer than the fragment length.

Unhybridized probe portions may be at either end of the probe (e.g., 3′ or 5′ end of a probe) or at both ends of the probe (i.e., 3′ and 5′ ends of a probe) and may comprise any number of monomers. In some embodiments, unhybridized probe portions may comprise about 1 to about 500 monomers. For example, unhybridized probe portions may comprise about 5, 10, 20, 30, 40, 50, 100, 200, 300 or 400 monomers.

In some embodiments, unhybridized probe portions may be removed from the target-probe species and/or reference-probe species, thereby generating trimmed probes. Removal of unhybridized probe portions may be achieved by any method known in the art for cleaving and/or digesting a polymer, such as, for example, a method for cleaving or digesting a single stranded nucleic acid. Unhybridized probe portions may be removed from the 5′ end of the probe and/or the 3′ end of the probe. Such methods may comprise the use of chemical and/or enzymatic cleavage or digestion. In some embodiments, an enzyme capable of cleaving phosphodiester bonds between nucleotide subunits of a nucleic acid is used for removing the unhybridized probe portions. Such enzymes may include, without limitation, nucleases (e.g., DNAse I, RNAse I), endonucleases (e.g., mung bean nuclease, S1 nuclease, and the like), restriction nucleases, exonucleases (e.g., Exonuclease I, Exonuclease III, Exonuclease T, T7 Exonuclease, Lambda Exonuclease, and the like), phosphodiesterases (e.g., Phosphodiesterase II, calf spleen phosphodiesterase, snake venom phosphodiesterase, and the like), deoxyribonucleases (DNAse), ribonucleases (RNAse), flap endonucleases, 5′ nucleases, 3′ nucleases, 3′-5′ exonucleases, 5′-3′ exonucleases and the like, or combinations thereof. Trimmed probes generally are of the same or substantially the same length as the fragment to which they hybridize. Thus, determining the length of a trimmed probe herein can provide a measurement of the corresponding nucleic acid fragment length. Trimmed probe length can be measured using any of the methods known in the art or described herein for determining nucleic acid fragment length. In some embodiments, probes may contain a detectable molecule or entity to facilitate detection and/or length determination (e.g., a fluorophore, radioisotope, colorimetric agent, particle, enzyme, and the like). Trimmed probe length may be assessed with or without separating products of unhybridized portions after they are removed.

In some embodiments, trimmed probes are dissociated (i.e., separated) from their corresponding nucleic acid fragments. Probes may be separated from their corresponding nucleic acid fragments using any method known in the art, including, but not limited to, heat or chemical denaturation. Trimmed probes can be distinguished from corresponding nucleic acid fragments by a method known in the art or described herein for labeling and/or isolating a species of molecule in a mixture. For example, a probe and/or nucleic acid fragment may comprise a detectable property such that a probe is distinguishable from the nucleic acid to which it hybridizes. Non-limiting examples of detectable properties include, optical properties, electrical properties, magnetic properties, chemical properties, and time and/or speed through an opening of known size. In some embodiments, probes and sample nucleic acid fragments are physically separated from each other. Separation can be accomplished, for example, using capture ligands, such as biotin or other affinity ligands, and capture agents, such as avidin, streptavidin, an antibody, or a receptor. A probe or nucleic acid fragment can contain a capture ligand having specific binding activity for a capture agent. For example, probes can be biotinylated or attached to an affinity ligand using methods well known in the art and separated away from sample nucleic acid fragments (or vice versa) using a pull-down assay with streptavidin-coated beads, for example. In some embodiments, a capture ligand and capture agent or any other moiety (e.g., mass tag) can be used to add mass to the nucleic acid fragments such that they can be excluded from the mass range of the probes detected in a mass spectrometer. In some embodiments, mass is added to the probes, by way of the monomers themselves and/or addition of a mass tag, to shift the mass range away from the mass range for the nucleic acid fragments.

Replication

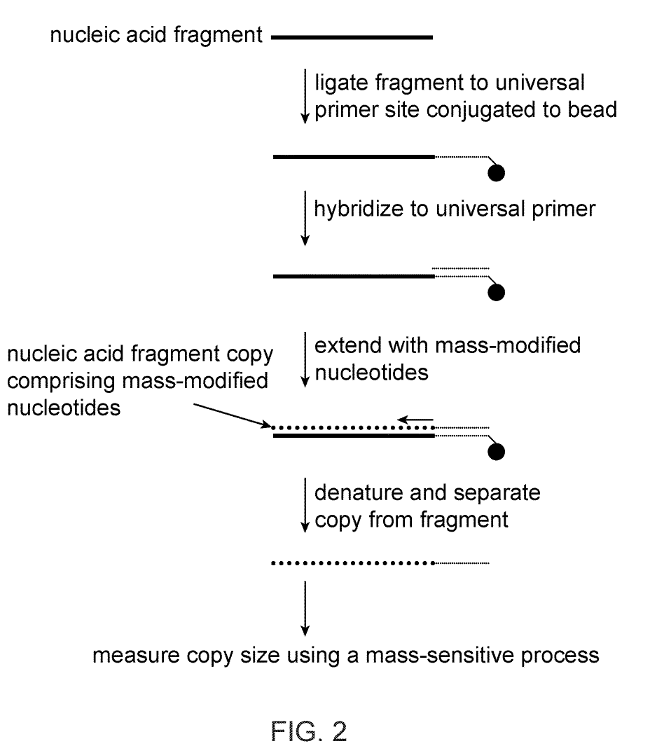

In some embodiments, nucleic acid fragment length is determined using a method whereby the fragment is replicated such that a fragment replica (e.g., complementary copy strand) comprises modified nucleotides, such as the mass-modified nucleotides described herein. The copy strand typically is of identical length as the original nucleic acid fragment (i.e., the copy strand and the original fragment comprise the same number of nucleotides). The copy strand, in some embodiments, may comprise mass modified nucleotides having substantially identical masses (i.e., mass equal nucleotides). An example method whereby fragment length is determined using a copy strand is shown in FIG. 2.

Generation of a copy strand can be performed using any nucleic acid replication technique known in the art. In some embodiments, a nucleic acid fragment of interest is ligated to a nucleotide sequence comprising a universal priming site which is capable of hybridizing to a universal primer. In some embodiments, a priming site and/or primer may comprise a label or binding partner useful for nucleic acid identification and/or separation, such as a binding partner described herein (e.g., biotin) or may be conjugated to a solid support, such as a solid support described herein (e.g., magnetic bead). In some embodiments, an extension reaction is performed on the primed fragment. Extension reactions can be performed, for example, using a polymerase that can incorporate mass-modified nucleotides (e.g., mass equal nucleotides) into a complementary copy strand of the nucleic acid fragment. In some embodiments, a denaturing step is performed. In some embodiments, a fragment and its copy are physically separated from each other. Methods for nucleic acid separation include separation methods known in the art and described herein (e.g., pull-down assays).

The size (e.g., mass) of a fragment copy comprising mass-modified nucleotides can be measured using a mass-sensitive process known in the art or described herein. In some embodiments, the length of a fragment can be determined based on the size (e.g., mass) of the copy. In some embodiments, the universal primer sequence is removed prior to mass measurement. In some embodiments, the mass of the universal primer sequence is subtracted from the mass measurement.

Mass Spectrometry

In some embodiments, mass spectrometry is used to determine nucleic acid fragment length. Mass spectrometry methods typically are used to determine the mass of a molecule, such as a nucleic acid fragment. In some embodiments, nucleic acid fragment length can be extrapolated from the mass of the fragment. In some embodiments, a predicted range of nucleic acid fragment lengths can be extrapolated from the mass of the fragment. In some embodiments, nucleic acid fragment length can be extrapolated from the mass of a probe that hybridizes to the fragment, such as a probe (e.g., trimmed probe) described herein. In some embodiments, presence of a target and/or reference nucleic acid of a given length can be verified by comparing the mass of the detected signal with the expected mass of the target and/or reference fragment. In some embodiments, the relative signal strength, e.g., mass peak on a spectra, for a particular nucleic acid fragment and/or fragment length can indicate the relative population of the fragment species amongst other nucleic acids in the sample (see e.g., Jurinke et al. (2004) Mol. Biotechnol. 26, 147-164).

Mass spectrometry generally works by ionizing chemical compounds to generate charged molecules or molecule fragments and measuring their mass-to-charge ratios. A typical mass spectrometry procedure involves several steps, including (1) loading a sample onto the mass spectrometry instrument followed by vaporization, (2) ionization of the sample components by any one of a variety of methods (e.g., impacting with an electron beam), resulting in charged particles (ions), (3) separation of ions according to their mass-to-charge ratio in an analyzer by electromagnetic fields, (4) detection of ions (e.g., by a quantitative method), and (5) processing of the ion signal into mass spectra.

Mass spectrometry methods are well known in the art (see, e.g., Burlingame et al. Anal. Chem. 70:647R-716R (1998)), and include, for example, quadrupole mass spectrometry, ion trap mass spectrometry, time-of-flight mass spectrometry, gas chromatography mass spectrometry and tandem mass spectrometry can be used with the methods described herein. The basic processes associated with a mass spectrometry method are the generation of gas-phase ions derived from the sample, and the measurement of their mass. The movement of gas-phase ions can be precisely controlled using electromagnetic fields generated in the mass spectrometer. The movement of ions in these electromagnetic fields is proportional to the m/z (mass to charge ratio) of the ion and this forms the basis of measuring the m/z and therefore the mass of a sample. The movement of ions in these electromagnetic fields allows for the containment and focusing of the ions which accounts for the high sensitivity of mass spectrometry. During the course of m/z measurement, ions are transmitted with high efficiency to particle detectors that record the arrival of these ions. The quantity of ions at each m/z is demonstrated by peaks on a graph where the x axis is m/z and the y axis is relative abundance. Different mass spectrometers have different levels of resolution, that is, the ability to resolve peaks between ions closely related in mass. The resolution is defined as R=m/delta m, where m is the ion mass and delta m is the difference in mass between two peaks in a mass spectrum. For example, a mass spectrometer with a resolution of 1000 can resolve an ion with a m/z of 100.0 from an ion with a m/z of 100.1. Certain mass spectrometry methods can utilize various combinations of ion sources and mass analyzers which allows for flexibility in designing customized detection protocols. In some embodiments, mass spectrometers can be programmed to transmit all ions from the ion source into the mass spectrometer either sequentially or at the same time. In some embodiments, a mass spectrometer can be programmed to select ions of a particular mass for transmission into the mass spectrometer while blocking other ions.

Several types of mass spectrometers are available or can be produced with various configurations. In general, a mass spectrometer has the following major components: a sample inlet, an ion source, a mass analyzer, a detector, a vacuum system, and instrument-control system, and a data system. Difference in the sample inlet, ion source, and mass analyzer generally define the type of instrument and its capabilities. For example, an inlet can be a capillary-column liquid chromatography source or can be a direct probe or stage such as used in matrix-assisted laser desorption. Common ion sources are, for example, electrospray, including nanospray and microspray or matrix-assisted laser desorption. Mass analyzers include, for example, a quadrupole mass filter, ion trap mass analyzer and time-of-flight mass analyzer.

The ion formation process is a starting point for mass spectrum analysis. Several ionization methods are available and the choice of ionization method depends on the sample used for analysis. For example, for the analysis of polypeptides a relatively gentle ionization procedure such as electrospray ionization (ESI) can be desirable. For ESI, a solution containing the sample is passed through a fine needle at high potential which creates a strong electrical field resulting in a fine spray of highly charged droplets that is directed into the mass spectrometer. Other ionization procedures include, for example, fast-atom bombardment (FAB) which uses a high-energy beam of neutral atoms to strike a solid sample causing desorption and ionization. Matrix-assisted laser desorption ionization (MALDI) is a method in which a laser pulse is used to strike a sample that has been crystallized in an UV-absorbing compound matrix (e.g., 2,5-dihydroxybenzoic acid, alpha-cyano-4-hydroxycinammic acid, 3-hydroxypicolinic acid (3-HPA), di-ammoniumcitrate (DAC) and combinations thereof). Other ionization procedures known in the art include, for example, plasma and glow discharge, plasma desorption ionization, resonance ionization, and secondary ionization.

A variety of mass analyzers are available that can be paired with different ion sources. Different mass analyzers have different advantages as known in the art and as described herein. The mass spectrometer and methods chosen for detection depends on the particular assay, for example, a more sensitive mass analyzer can be used when a small amount of ions are generated for detection. Several types of mass analyzers and mass spectrometry methods are described below.

Ion mobility mass (IM) spectrometry is a gas-phase separation method. IM separates gas-phase ions based on their collision cross-section and can be coupled with time-of-flight (TOF) mass spectrometry. IM-MS is discussed in more detail by Verbeck et al. in the Journal of Biomolecular Techniques (Vol 13, Issue 2, 56-61).

Quadrupole mass spectrometry utilizes a quadrupole mass filter or analyzer. This type of mass analyzer is composed of four rods arranged as two sets of two electrically connected rods. A combination of rf and dc voltages are applied to each pair of rods which produces fields that cause an oscillating movement of the ions as they move from the beginning of the mass filter to the end. The result of these fields is the production of a high-pass mass filter in one pair of rods and a low-pass filter in the other pair of rods. Overlap between the high-pass and low-pass filter leaves a defined m/z that can pass both filters and traverse the length of the quadrupole. This m/z is selected and remains stable in the quadrupole mass filter while all other m/z have unstable trajectories and do not remain in the mass filter. A mass spectrum results by ramping the applied fields such that an increasing m/z is selected to pass through the mass filter and reach the detector. In addition, quadrupoles can also be set up to contain and transmit ions of all m/z by applying a rf-only field. This allows quadrupoles to function as a lens or focusing system in regions of the mass spectrometer where ion transmission is needed without mass filtering.

A quadrupole mass analyzer, as well as the other mass analyzers described herein, can be programmed to analyze a defined m/z or mass range. Since the desired mass range of nucleic acid fragment is known, in some embodiments, a mass spectrometer can be programmed to transmit ions of the projected correct mass range while excluding ions of a higher or lower mass range. The ability to select a mass range can decrease the background noise in the assay and thus increase the signal-to-noise ratio. Thus, in some embodiments, a mass spectrometer can accomplish a separation step as well as detection and identification of certain mass-distinguishable nucleic acid fragments.

Ion trap mass spectrometry utilizes an ion trap mass analyzer. Typically, fields are applied such that ions of all m/z are initially trapped and oscillate in the mass analyzer. Ions enter the ion trap from the ion source through a focusing device such as an octapole lens system. Ion trapping takes place in the trapping region before excitation and ejection through an electrode to the detector. Mass analysis can be accomplished by sequentially applying voltages that increase the amplitude of the oscillations in a way that ejects ions of increasing m/z out of the trap and into the detector. In contrast to quadrupole mass spectrometry, all ions are retained in the fields of the mass analyzer except those with the selected m/z. Control of the number of ions can be accomplished by varying the time over which ions are injected into the trap.

Time-of-flight mass spectrometry utilizes a time-of-flight mass analyzer. Typically, an ion is first given a fixed amount of kinetic energy by acceleration in an electric field (generated by high voltage). Following acceleration, the ion enters a field-free or “drift” region where it travels at a velocity that is inversely proportional to its m/z. Therefore, ions with low m/z travel more rapidly than ions with high m/z. The time required for ions to travel the length of the field-free region is measured and used to calculate the m/z of the ion.

Gas chromatography mass spectrometry often can a target in real-time. The gas chromatography (GC) portion of the system separates the chemical mixture into pulses of analyte and the mass spectrometer (MS) identifies and quantifies the analyte.

Tandem mass spectrometry can utilize combinations of the mass analyzers described above. Tandem mass spectrometers can use a first mass analyzer to separate ions according to their m/z in order to isolate an ion of interest for further analysis. The isolated ion of interest is then broken into fragment ions (called collisionally activated dissociation or collisionally induced dissociation) and the fragment ions are analyzed by the second mass analyzer. These types of tandem mass spectrometer systems are called tandem in space systems because the two mass analyzers are separated in space, usually by a collision cell. Tandem mass spectrometer systems also include tandem in time systems where one mass analyzer is used, however the mass analyzer is used sequentially to isolate an ion, induce fragmentation, and then perform mass analysis.

Mass spectrometers in the tandem in space category have more than one mass analyzer. For example, a tandem quadrupole mass spectrometer system can have a first quadrupole mass filter, followed by a collision cell, followed by a second quadrupole mass filter and then the detector. Another arrangement is to use a quadrupole mass filter for the first mass analyzer and a time-of-flight mass analyzer for the second mass analyzer with a collision cell separating the two mass analyzers. Other tandem systems are known in the art including reflectron-time-of-flight, tandem sector and sector-quadrupole mass spectrometry.

Mass spectrometers in the tandem in time category have one mass analyzer that performs different functions at different times. For example, an ion trap mass spectrometer can be used to trap ions of all m/z. A series of rf scan functions are applied which ejects ions of all m/z from the trap except the m/z of ions of interest. After the m/z of interest has been isolated, an rf pulse is applied to produce collisions with gas molecules in the trap to induce fragmentation of the ions. Then the m/z values of the fragmented ions are measured by the mass analyzer. Ion cyclotron resonance instruments, also known as Fourier transform mass spectrometers, are an example of tandem-in-time systems.

Several types of tandem mass spectrometry experiments can be performed by controlling the ions that are selected in each stage of the experiment. The different types of experiments utilize different modes of operation, sometimes called “scans,” of the mass analyzers. In a first example, called a mass spectrum scan, the first mass analyzer and the collision cell transmit all ions for mass analysis into the second mass analyzer. In a second example, called a product ion scan, the ions of interest are mass-selected in the first mass analyzer and then fragmented in the collision cell. The ions formed are then mass analyzed by scanning the second mass analyzer. In a third example, called a precursor ion scan, the first mass analyzer is scanned to sequentially transmit the mass analyzed ions into the collision cell for fragmentation. The second mass analyzer mass-selects the product ion of interest for transmission to the detector. Therefore, the detector signal is the result of all precursor ions that can be fragmented into a common product ion. Other experimental formats include neutral loss scans where a constant mass difference is accounted for in the mass scans.

For quantification, controls may be used which can provide a signal in relation to the amount of the nucleic acid fragment, for example, that is present or is introduced. A control to allow conversion of relative mass signals into absolute quantities can be accomplished by addition of a known quantity of a mass tag or mass label to each sample before detection of the nucleic acid fragments. See for example, Ding and Cantor (2003) Proc Natl Acad Sci USA. March 18; 100(6):3059-64. Any mass tag that does not interfere with detection of the fragments can be used for normalizing the mass signal. Such standards typically have separation properties that are different from those of any of the molecular tags in the sample, and could have the same or different mass signatures.

In some embodiments, a separation step can be used to remove salts, enzymes, or other buffer components from the nucleic acid sample. Several methods well known in the art, such as chromatography, gel electrophoresis, or precipitation, can be used to clean up the sample. For example, size exclusion chromatography or affinity chromatography can be used to remove salt from a sample. The choice of separation method can depend on the amount of a sample. For example, when small amounts of sample are available or a miniaturized apparatus is used, a micro-affinity chromatography separation step can be used. In addition, whether a separation step is desired, and the choice of separation method, can depend on the detection method used. In some embodiments, salts can absorb energy from the laser in matrix-assisted laser desorption/ionization and result in lower ionization efficiency. Thus, the efficiency of matrix-assisted laser desorption/ionization and electrospray ionization sometimes can be improved by removing salts from a sample.

Electrophoresis

In some embodiments, electrophoresis is used to determine nucleic acid fragment length. In some embodiments, electrophoresis is not used to determine nucleic acid fragment length. In some embodiments, length of a corresponding probe (e.g., a corresponding trimmed probe described herein) is determined using electrophoresis. Electrophoresis also can be used, in some embodiments, as a length-based separation method as described herein. Any electrophoresis method known in the art, whereby nucleic acids are separated by length, can be used in conjunction with the methods provided herein, which include, but are not limited to, standard electrophoretic techniques and specialized electrophoretic techniques, such as, for example capillary electrophoresis. Examples of methods for separating nucleic acid and measuring nucleic acid fragment length using standard electrophoretic techniques can be found in the art. A non-limiting example is presented herein. After running a nucleic acid sample in an agarose or polyacrylamide gel, the gel may be labeled (e.g., stained) with ethidium bromide (see, Sambrook and Russell, Molecular Cloning: A Laboratory Manual 3d ed., 2001). The presence of a band of the same size as a standard control is an indication of the presence of a particular nucleic acid sequence length, the amount of which may then, in some embodiments, be compared to the control based on the intensity of the band, thus detecting and quantifying the nucleic acid sequence length of interest.

In some embodiments, capillary electrophoresis is used to separate, identify and sometimes quantify nucleic acid fragments or probes. Capillary electrophoresis (CE) encompasses a family of related separation techniques that use narrow-bore fused-silica capillaries to separate a complex array of large and small molecules, such as, for example, nucleic acids of varying length. High electric field strengths can be used to separate nucleic acid molecules based on differences in charge, size and hydrophobicity. Sample introduction is accomplished by immersing the end of the capillary into a sample vial and applying pressure, vacuum or voltage. Depending on the types of capillary and electrolytes used, the technology of CE can be segmented into several separation techniques, any of which can be adapted to the methods provided herein. Non-limiting examples of these include Capillary Zone Electrophoresis (CZE), also known as free-solution CE (FSCE), Capillary Isoelectric Focusing (CIEF), Isotachophoresis (ITP), Electrokinetic Chromatography (EKC), Micellar Electrokinetic Capillary Chromatography (MECC OR MEKC), Micro Emulsion Electrokinetic Chromatography (MEEKC), Non-Aqueous Capillary Electrophoresis (NACE), and Capillary Electrochromatography (CEC).

Any device, instrument or machine capable of performing capillary electrophoresis can be used in conjunction with the methods provided herein. In general, a capillary electrophoresis system's main components are a sample vial, source and destination vials, a capillary, electrodes, a high-voltage power supply, a detector, and a data output and handling device. The source vial, destination vial and capillary are filled with an electrolyte such as an aqueous buffer solution. To introduce the sample, the capillary inlet is placed into a vial containing the sample and then returned to the source vial (sample is introduced into the capillary via capillary action, pressure, or siphoning). The migration of the analytes (i.e. nucleic acids) is then initiated by an electric field that is applied between the source and destination vials and is supplied to the electrodes by the high-voltage power supply. Ions, positive or negative, are pulled through the capillary in the same direction by electroosmotic flow. The analytes (i.e. nucleic acids) separate as they migrate due to their electrophoretic mobility and are detected near the outlet end of the capillary. The output of the detector is sent to a data output and handling device such as an integrator or computer. The data is then displayed as an electropherogram, which can report detector response as a function of time. Separated nucleic acids can appear as peaks with different migration times in an electropherogram.

Separation by capillary electrophoresis can be detected by several detection devices. The majority of commercial systems use UV or UV-Vis absorbance as their primary mode of detection. In these systems, a section of the capillary itself is used as the detection cell. The use of on-tube detection enables detection of separated analytes with no loss of resolution. In general, capillaries used in capillary electrophoresis can be coated with a polymer for increased stability. The portion of the capillary used for UV detection is often optically transparent. The path length of the detection cell in capillary electrophoresis (˜50 micrometers) is far less than that of a traditional UV cell (˜1 cm). According to the Beer-Lambert law, the sensitivity of the detector is proportional to the path length of the cell. To improve the sensitivity, the path length can be increased, though this can result in a loss of resolution. The capillary tube itself can be expanded at the detection point, creating a “bubble cell” with a longer path length or additional tubing can be added at the detection point. Both of these methods, however, may decrease the resolution of the separation.

Fluorescence detection also can be used in capillary electrophoresis for samples that naturally fluoresce or are chemically modified to contain fluorescent tags, such as, for example, labeled nucleic acid fragments or probes described herein. This mode of detection offers high sensitivity and improved selectivity for these samples. The method requires that the light beam be focused on the capillary. Laser-induced fluorescence can be been used in CE systems with detection limits as low as 10-18 to 10-21 mol. The sensitivity of the technique is attributed to the high intensity of the incident light and the ability to accurately focus the light on the capillary.

Several capillary electrophoresis machines are known in the art and can be used in conjunction with the methods provided herein. These include, but are not limited to, CALIPER LAB CHIP GX (Caliper Life Sciences, Mountain View, Calif.), P/ACE 2000 Series (Beckman Coulter, Brea, Calif.), HP G1600A CE (Hewlett-Packard, Palo Alto, Calif.), AGILENT 7100 CE (Agilent Technologies, Santa Clara, Calif.), and ABI PRISM Genetic Analyzer (Applied Biosystems, Carlsbad, Calif.).

Microscopy

In some embodiments, nucleic acid fragment length is determined using an imaging-based method, such as a microscopy method. In some embodiments, length of a corresponding probe (e.g., a corresponding trimmed probe described herein) is determined using an imaging-based method. In some embodiments, fragment length or corresponding probe length can be determined by microscopic visualization of single nucleic acid fragments or probes (see e.g., U.S. Pat. No. 5,720,928). In some embodiments, nucleic acid fragments or probes are fixed to a surface (e.g., modified glass surface) in an elongated state, stained and visualized microscopically. Images of the fragments or probes can be collected and processed (e.g., measured for length). In some embodiments, imaging and image analysis steps can be automated. Methods for directly visualizing nucleic acid fragments or probes using microscopy are known in the art (see e.g., Lai et al. (1999) Nat Genet. 23(3):309-13; Aston et al. (1999) Trends Biotechnol. 17(7):297-302; Aston et al. (1999) Methods Enzymol. 303:55-73; Jing et al. (1998) Proc Natl Acad Sci USA. 95(14):8046-51; and U.S. Pat. No. 5,720,928). Other microscopy methods that can be used with the methods described herein include, without limitation, scanning tunneling microscopy (STM), atomic force microscopy (ATM), scanning force microscopy (SFM), photon scanning microscopy (PSTM), scanning tunneling potentiometry (STP), magnetic force microscopy (MFM), scanning probe microscopy, scanning voltage microscopy, photoconductive atomic force microscopy, electrochemical scanning tunneling microscopy, electron microscopy, spin polarized scanning tunneling microscopy (SPSTM), scanning thermal microscopy, scanning joule expansion microscopy, photothermal microspectroscopy, and the like.

In some embodiments, scanning tunneling microscopy (STM) can be used to determine nucleic acid fragment or probe length. STM methods often can generate atomic-level images of molecules, such as nucleic acid fragments or probes. STM can be performed, for example, in air, water, ultra-high vacuum, various other liquid or gas ambients, and can be performed at temperatures ranging from near zero Kelvin to a few hundred degrees Celsius, for example. The components of an STM system typically include scanning tip, piezoelectric controlled height and x, y scanner, coarse sample-to-tip control, vibration isolation system, and computer. STM methods are generally based on the concept of quantum tunneling. For example, when a conducting tip is brought close to the surface of a molecule (e.g., nucleic acid fragment), a bias (i.e., voltage difference) applied between the two can allow electrons to tunnel through the vacuum between them. The resulting tunneling current is a function of tip position, applied voltage, and the local density of states (LDOS) of the sample. Information is acquired by monitoring the current as the tip's position scans across the surface, and can be displayed in image form. If the tip is moved across the sample in the x-y plane, the changes in surface height and density of states cause changes in current. These changes can be mapped in images. In some embodiments, the change in current with respect to position can be measured itself, or the height, z, of the tip corresponding to a constant current can be measured. These two modes often are referred to as constant height mode and constant current mode, respectively.

In some embodiments, atomic force microscopy (AFM) can be used to determine nucleic acid fragment or probe length. AFM generally is a high-resolution type of nanoscale microscopy. Information about an object (e.g., nucleic acid fragment or probe) typically is gathered by “feeling” the surface with a mechanical probe. Piezoelectric elements that facilitate tiny but accurate and precise movements on electronic command can facilitate very precise scanning. In some variations, electric potentials can be scanned using conducting cantilevers. The components of an AFM system typically include a cantilever with a sharp tip (i.e., probe) at its end that is used to scan the surface of a specimen (e.g., nucleic acid fragment). The cantilever typically is silicon or silicon nitride with a tip radius of curvature on the order of nanometers. When the tip is brought into proximity of a sample surface, forces between the tip and the sample lead to a deflection of the cantilever according to Hooke's law. Depending on the situation, forces that are measured in AFM include, for example, mechanical contact force, van der Waals forces, capillary forces, chemical bonding, electrostatic forces, magnetic forces, Casimir forces, solvation forces, and the like. Typically, the deflection is measured using a laser spot reflected from the top surface of the cantilever into an array of photodiodes. Other methods that are used include optical interferometry, capacitive sensing or piezoresistive AFM cantilevers.

Nanopore

In some embodiments, nucleic acid fragment length is determined using a nanopore. In some embodiments, length of a corresponding probe (e.g., a corresponding trimmed probe described herein) is determined using a nanopore. A nanopore is a small hole or channel, typically of the order of 1 nanometer in diameter. Certain transmembrane cellular proteins can act as nanopores (e.g., alpha-hemolysin). In some embodiments, nanopores can be synthesized (e.g., using a silicon platform). Immersion of a nanopore in a conducting fluid and application of a potential across it results in a slight electrical current due to conduction of ions through the nanopore. The amount of current which flows is sensitive to the size of the nanopore. As a nucleic acid fragment or probe passes through a nanopore, the nucleic acid molecule obstructs the nanopore to a certain degree and generates a change to the current. The duration of current change as the nucleic acid fragment or probe passes through the nanopore can be measured. In some embodiments, nucleic acid fragment or probe length can be determined based on this measurement.

In some embodiments, nucleic acid fragment or probe length may be determined as a function of time. In some embodiments, longer nucleic acid fragments or probes may take relatively more time to pass through a nanopore and shorter nucleic acid fragments or probes may take relatively less time to pass through a nanopore. Thus, relative length of a fragment or probe can be determined based on nanopore transit time, in some embodiments. In some embodiments, approximate or absolute fragment or probe length can be determined by comparing nanopore transit time of fragments or probes to transit times for a set of standards (i.e., with known lengths).

### Determination of Fragment Sequence

In some embodiments, nucleic acids (e.g., target fragments, reference fragments) may be sequenced. In some embodiments, a nucleic acid is not sequenced, and the sequence of a nucleic acid is not determined by a sequencing method, when performing a method described herein. In some embodiments, a full or substantially full sequence is obtained and sometimes a partial sequence is obtained. In some embodiments, fragment length is determined using a sequencing method. In some embodiments, fragment length is determined without use of a sequencing method. Any sequencing method suitable for conducting methods described herein can be utilized. In some embodiments, a high-throughput sequencing method is used. High-throughput sequencing methods generally involve clonally amplified DNA templates or single DNA molecules that are sequenced in a massively parallel fashion within a flow cell (e.g. as described in Metzker M Nature Rev 11:31-46 (2010); Volkerding et al. Clin Chem 55:641-658 (2009)). Such sequencing methods also can provide digital quantitative information, where each sequence read is a “count” representing an individual clonal DNA template or a single DNA molecule. High-throughput sequencing technologies include, for example, sequencing-by-synthesis with reversible dye terminators, sequencing by oligonucleotide probe ligation, pyrosequencing and real time sequencing.

Systems utilized for high-throughput sequencing methods are commercially available and include, for example, the Roche 454 platform, the Applied Biosystems SOLID platform, the Helicos True Single Molecule DNA sequencing technology, the sequencing-by-hybridization platform from Affymetrix Inc., the single molecule, real-time (SMRT) technology of Pacific Biosciences, the sequencing-by-synthesis platforms from 454 Life Sciences, Illumina/Solexa and Helicos Biosciences, and the sequencing-by-ligation platform from Applied Biosystems. The ION TORRENT technology from Life technologies and nanopore sequencing also can be used in high-throughput sequencing approaches.

In some embodiments, first generation technology, such as, for example, Sanger sequencing including the automated Sanger sequencing, can be used in the methods provided herein. Additional sequencing technologies that include the use of developing nucleic acid imaging technologies (e.g. transmission electron microscopy (TEM) and atomic force microscopy (AFM)), also are contemplated herein. Examples of various sequencing technologies are described below.

A nucleic acid sequencing technology that may be used in the methods described herein is sequencing-by-synthesis and reversible terminator-based sequencing (e.g., Illumina's Genome Analyzer and Genome Analyzer II). With this technology, millions of nucleic acid (e.g., DNA) fragments can be sequenced in parallel. In one example of this type of sequencing technology, a flow cell is used which contains an optically transparent slide with 8 individual lanes on the surfaces of which are bound oligonucleotide anchors (e.g., adaptor primers). A flow cell often is a solid support that can be configured to retain and/or allow the orderly passage of reagent solutions over bound analytes. Flow cells frequently are planar in shape, optically transparent, generally in the millimeter or sub-millimeter scale, and often have channels or lanes in which the analyte/reagent interaction occurs.

In certain sequencing by synthesis procedures, for example, template DNA (e.g., circulating cell-free DNA (ccfDNA)) sometimes is fragmented into lengths of several hundred base pairs in preparation for library generation. In some embodiments, library preparation can be performed without further fragmentation or size selection of the template DNA (e.g., ccfDNA). Sample isolation and library generation may be performed using automated methods and apparatus, in certain embodiments. Briefly, template DNA is end repaired by a fill-in reaction, exonuclease reaction or a combination of a fill-in reaction and exonuclease reaction. The resulting blunt-end repaired template DNA is extended by a single nucleotide, which is complementary to a single nucleotide overhang on the 3′ end of an adapter primer, and often increases ligation efficiency. Any complementary nucleotides can be used for the extension/overhang nucleotides (e.g., A/T, C/G), however adenine frequently is used to extend the end-repaired DNA, and thymine often is used as the 3′ end overhang nucleotide.

In certain sequencing by synthesis procedures, for example, adapter oligonucleotides are complementary to the flow-cell anchors, and sometimes are utilized to associate the modified template DNA (e.g., end-repaired and single nucleotide extended) with a solid support, such as the inside surface of a flow cell, for example. In some embodiments, the adapter also includes identifiers (i.e., indexing nucleotides, or “barcode” nucleotides (e.g., a unique sequence of nucleotides usable as an identifier to allow unambiguous identification of a sample and/or chromosome)), one or more sequencing primer hybridization sites (e.g., sequences complementary to universal sequencing primers, single end sequencing primers, paired end sequencing primers, multiplexed sequencing primers, and the like), or combinations thereof (e.g., adapter/sequencing, adapter/identifier, adapter/identifier/sequencing). Identifiers or nucleotides contained in an adapter often are six or more nucleotides in length, and frequently are positioned in the adaptor such that the identifier nucleotides are the first nucleotides sequenced during the sequencing reaction. In certain embodiments, identifier nucleotides are associated with a sample but are sequenced in a separate sequencing reaction to avoid compromising the quality of sequence reads. Subsequently, the reads from the identifier sequencing and the DNA template sequencing are linked together and the reads de-multiplexed. After linking and de-multiplexing the sequence reads and/or identifiers can be further adjusted or processed as described herein.

In certain sequencing by synthesis procedures, utilization of identifiers allows multiplexing of sequence reactions in a flow cell lane, thereby allowing analysis of multiple samples per flow cell lane. The number of samples that can be analyzed in a given flow cell lane often is dependent on the number of unique identifiers utilized during library preparation and/or probe design. Non limiting examples of commercially available multiplex sequencing kits include Illumina's multiplexing sample preparation oligonucleotide kit and multiplexing sequencing primers and PhiX control kit (e.g., Illumina's catalog numbers PE-400-1001 and PE-400-1002, respectively). The methods described herein can be performed using any number of unique identifiers (e.g., 4, 8, 12, 24, 48, 96, or more). The greater the number of unique identifiers, the greater the number of samples and/or chromosomes, for example, that can be multiplexed in a single flow cell lane. Multiplexing using 12 identifiers, for example, allows simultaneous analysis of 96 samples (e.g., equal to the number of wells in a 96 well microwell plate) in an 8 lane flow cell. Similarly, multiplexing using 48 identifiers, for example, allows simultaneous analysis of 384 samples (e.g., equal to the number of wells in a 384 well microwell plate) in an 8 lane flow cell.

In certain sequencing by synthesis procedures, adapter-modified, single-stranded template DNA is added to the flow cell and immobilized by hybridization to the anchors under limiting-dilution conditions. In contrast to emulsion PCR, DNA templates are amplified in the flow cell by “bridge” amplification, which relies on captured DNA strands “arching” over and hybridizing to an adjacent anchor oligonucleotide. Multiple amplification cycles convert the single-molecule DNA template to a clonally amplified arching “cluster,” with each cluster containing approximately 1000 clonal molecules. Approximately 50×10⁶ separate clusters can be generated per flow cell. For sequencing, the clusters are denatured, and a subsequent chemical cleavage reaction and wash leave only forward strands for single-end sequencing. Sequencing of the forward strands is initiated by hybridizing a primer complementary to the adapter sequences, which is followed by addition of polymerase and a mixture of four differently colored fluorescent reversible dye terminators. The terminators are incorporated according to sequence complementarity in each strand in a clonal cluster. After incorporation, excess reagents are washed away, the clusters are optically interrogated, and the fluorescence is recorded. With successive chemical steps, the reversible dye terminators are unblocked, the fluorescent labels are cleaved and washed away, and the next sequencing cycle is performed. This iterative, sequencing-by-synthesis process sometimes requires approximately 2.5 days to generate read lengths of 36 bases. With 50×10⁶ clusters per flow cell, the overall sequence output can be greater than 1 billion base pairs (Gb) per analytical run.

Another nucleic acid sequencing technology that may be used with the methods described herein is 454 sequencing (Roche). 454 sequencing uses a large-scale parallel pyrosequencing system capable of sequencing about 400-600 megabases of DNA per run. The process typically involves two steps. In the first step, sample nucleic acid (e.g. DNA) is sometimes fractionated into smaller fragments (300-800 base pairs) and polished (made blunt at each end). Short adaptors are then ligated onto the ends of the fragments. These adaptors provide priming sequences for both amplification and sequencing of the sample-library fragments. One adaptor (Adaptor B) contains a 5′-biotin tag for immobilization of the DNA library onto streptavidin-coated beads. After nick repair, the non-biotinylated strand is released and used as a single-stranded template DNA (sstDNA) library. The sstDNA library is assessed for its quality and the optimal amount (DNA copies per bead) needed for emPCR is determined by titration. The sstDNA library is immobilized onto beads. The beads containing a library fragment carry a single sstDNA molecule. The bead-bound library is emulsified with the amplification reagents in a water-in-oil mixture. Each bead is captured within its own microreactor where PCR amplification occurs. This results in bead-immobilized, clonally amplified DNA fragments.

In the second step of 454 sequencing, single-stranded template DNA library beads are added to an incubation mix containing DNA polymerase and are layered with beads containing sulfurylase and luciferase onto a device containing pico-liter sized wells. Pyrosequencing is performed on each DNA fragment in parallel. Addition of one or more nucleotides generates a light signal that is recorded by a CCD camera in a sequencing instrument. The signal strength is proportional to the number of nucleotides incorporated. Pyrosequencing exploits the release of pyrophosphate (PPi) upon nucleotide addition. PPi is converted to ATP by ATP sulfurylase in the presence of adenosine 5′ phosphosulfate. Luciferase uses ATP to convert luciferin to oxyluciferin, and this reaction generates light that is discerned and analyzed (see, for example, Margulies, M. et al. Nature 437:376-380 (2005)).

Another nucleic acid sequencing technology that may be used in the methods provided herein is Applied Biosystems' SOLiD™ technology. In SOLiD™ sequencing-by-ligation, a library of nucleic acid fragments is prepared from the sample and is used to prepare clonal bead populations. With this method, one species of nucleic acid fragment will be present on the surface of each bead (e.g. magnetic bead). Sample nucleic acid (e.g. genomic DNA) is sheared into fragments, and adaptors are subsequently attached to the 5′ and 3′ ends of the fragments to generate a fragment library. The adapters are typically universal adapter sequences so that the starting sequence of every fragment is both known and identical. Emulsion PCR takes place in microreactors containing all the necessary reagents for PCR. The resulting PCR products attached to the beads are then covalently bound to a glass slide. Primers then hybridize to the adapter sequence within the library template. A set of four fluorescently labeled di-base probes compete for ligation to the sequencing primer. Specificity of the di-base probe is achieved by interrogating every 1st and 2nd base in each ligation reaction. Multiple cycles of ligation, detection and cleavage are performed with the number of cycles determining the eventual read length. Following a series of ligation cycles, the extension product is removed and the template is reset with a primer complementary to the n−1 position for a second round of ligation cycles. Often, five rounds of primer reset are completed for each sequence tag. Through the primer reset process, each base is interrogated in two independent ligation reactions by two different primers. For example, the base at read position 5 is assayed by primer number 2 in ligation cycle 2 and by primer number 3 in ligation cycle 1.

Another nucleic acid sequencing technology that may be used in the methods described herein is the Helicos True Single Molecule Sequencing (tSMS). In the tSMS technique, a polyA sequence is added to the 3′ end of each nucleic acid (e.g. DNA) strand from the sample. Each strand is labeled by the addition of a fluorescently labeled adenosine nucleotide. The DNA strands are then hybridized to a flow cell, which contains millions of oligo-T capture sites that are immobilized to the flow cell surface. The templates can be at a density of about 100 million templates/cm². The flow cell is then loaded into a sequencing apparatus and a laser illuminates the surface of the flow cell, revealing the position of each template. A CCD camera can map the position of the templates on the flow cell surface. The template fluorescent label is then cleaved and washed away. The sequencing reaction begins by introducing a DNA polymerase and a fluorescently labeled nucleotide. The oligo-T nucleic acid serves as a primer. The polymerase incorporates the labeled nucleotides to the primer in a template directed manner. The polymerase and unincorporated nucleotides are removed. The templates that have directed incorporation of the fluorescently labeled nucleotide are detected by imaging the flow cell surface. After imaging, a cleavage step removes the fluorescent label, and the process is repeated with other fluorescently labeled nucleotides until the desired read length is achieved. Sequence information is collected with each nucleotide addition step (see, for example, Harris T. D. et al., Science 320:106-109 (2008)).

Another nucleic acid sequencing technology that may be used in the methods provided herein is the single molecule, real-time (SMRT™) sequencing technology of Pacific Biosciences. With this method, each of the four DNA bases is attached to one of four different fluorescent dyes. These dyes are phospholinked. A single DNA polymerase is immobilized with a single molecule of template single stranded DNA at the bottom of a zero-mode waveguide (ZMW). A ZMW is a confinement structure which enables observation of incorporation of a single nucleotide by DNA polymerase against the background of fluorescent nucleotides that rapidly diffuse in an out of the ZMW (in microseconds). It takes several milliseconds to incorporate a nucleotide into a growing strand. During this time, the fluorescent label is excited and produces a fluorescent signal, and the fluorescent tag is cleaved off. Detection of the corresponding fluorescence of the dye indicates which base was incorporated. The process is then repeated.

Another nucleic acid sequencing technology that may be used in the methods described herein is ION TORRENT (Life Technologies) single molecule sequencing which pairs semiconductor technology with a simple sequencing chemistry to directly translate chemically encoded information (A, C, G, T) into digital information (0, 1) on a semiconductor chip. ION TORRENT uses a high-density array of micro-machined wells to perform nucleic acid sequencing in a massively parallel way. Each well holds a different DNA molecule. Beneath the wells is an ion-sensitive layer and beneath that an ion sensor. Typically, when a nucleotide is incorporated into a strand of DNA by a polymerase, a hydrogen ion is released as a byproduct. If a nucleotide, for example a C, is added to a DNA template and is then incorporated into a strand of DNA, a hydrogen ion will be released. The charge from that ion will change the pH of the solution, which can be detected by an ion sensor. A sequencer can call the base, going directly from chemical information to digital information. The sequencer then sequentially floods the chip with one nucleotide after another. If the next nucleotide that floods the chip is not a match, no voltage change will be recorded and no base will be called. If there are two identical bases on the DNA strand, the voltage will be double, and the chip will record two identical bases called. Because this is direct detection (i.e. detection without scanning, cameras or light), each nucleotide incorporation is recorded in seconds.

Another nucleic acid sequencing technology that may be used in the methods described herein is the chemical-sensitive field effect transistor (CHEMFET) array. In one example of this sequencing technique, DNA molecules are placed into reaction chambers, and the template molecules can be hybridized to a sequencing primer bound to a polymerase. Incorporation of one or more triphosphates into a new nucleic acid strand at the 3′ end of the sequencing primer can be detected by a change in current by a CHEMFET sensor. An array can have multiple CHEMFET sensors. In another example, single nucleic acids are attached to beads, and the nucleic acids can be amplified on the bead, and the individual beads can be transferred to individual reaction chambers on a CHEMFET array, with each chamber having a CHEMFET sensor, and the nucleic acids can be sequenced (see, for example, U.S. Patent Application Publication No. 2009/0026082).

Another nucleic acid sequencing technology that may be used in the methods described herein is electron microscopy. In one example of this sequencing technique, individual nucleic acid (e.g. DNA) molecules are labeled using metallic labels that are distinguishable using an electron microscope. These molecules are then stretched on a flat surface and imaged using an electron microscope to measure sequences (see, for example, Moudrianakis E. N. and Beer M. Proc Natl Acad Sci USA. 1965 March; 53:564-71). In some embodiments, transmission electron microscopy (TEM) is used (e.g. Halcyon Molecular's TEM method). This method, termed Individual Molecule Placement Rapid Nano Transfer (IMPRNT), includes utilizing single atom resolution transmission electron microscope imaging of high-molecular weight (e.g. about 150 kb or greater) DNA selectively labeled with heavy atom markers and arranging these molecules on ultra-thin films in ultra-dense (3 nm strand-to-strand) parallel arrays with consistent base-to-base spacing. The electron microscope is used to image the molecules on the films to determine the position of the heavy atom markers and to extract base sequence information from the DNA (see, for example, International Patent Application Publication No. WO2009/046445).

Other sequencing methods that may be used to conduct methods herein include digital PCR and sequencing by hybridization. Digital polymerase chain reaction (digital PCR or dPCR) can be used to directly identify and quantify nucleic acids in a sample. Digital PCR can be performed in an emulsion, in some embodiments. For example, individual nucleic acids are separated, e.g., in a microfluidic chamber device, and each nucleic acid is individually amplified by PCR. Nucleic acids can be separated such that there is no more than one nucleic acid per well. In some embodiments, different probes can be used to distinguish various alleles (e.g. fetal alleles and maternal alleles). Alleles can be enumerated to determine copy number. In sequencing by hybridization, the method involves contacting a plurality of polynucleotide sequences with a plurality of polynucleotide probes, where each of the plurality of polynucleotide probes can be optionally tethered to a substrate. The substrate can be a flat surface with an array of known nucleotide sequences, in some embodiments. The pattern of hybridization to the array can be used to determine the polynucleotide sequences present in the sample. In some embodiments, each probe is tethered to a bead, e.g., a magnetic bead or the like. Hybridization to the beads can be identified and used to identify the plurality of polynucleotide sequences within the sample.

In some embodiments, nanopore sequencing can be used in the methods described herein. Nanopore sequencing is a single-molecule sequencing technology whereby a single nucleic acid molecule (e.g. DNA) is sequenced directly as it passes through a nanopore. A nanopore is a small hole or channel, of the order of 1 nanometer in diameter. Certain transmembrane cellular proteins can act as nanopores (e.g. alpha-hemolysin). In some embodiments, nanopores can be synthesized (e.g. using a silicon platform). Immersion of a nanopore in a conducting fluid and application of a potential across it results in a slight electrical current due to conduction of ions through the nanopore. The amount of current which flows is sensitive to the size of the nanopore. As a DNA molecule passes through a nanopore, each nucleotide on the DNA molecule obstructs the nanopore to a different degree and generates characteristic changes to the current. The amount of current which can pass through the nanopore at any given moment therefore varies depending on whether the nanopore is blocked by an A, a C, a G, a T, or in some embodiments, methyl-C. The change in the current through the nanopore as the DNA molecule passes through the nanopore represents a direct reading of the DNA sequence. In some embodiments a nanopore can be used to identify individual DNA bases as they pass through the nanopore in the correct order (see, for example, Soni G V and Meller A. Clin Chem 53: 1996-2001 (2007); International Application Publication No. WO2010/004265).

There are a number of ways that nanopores can be used to sequence nucleic acid molecules. In some embodiments, an exonuclease enzyme, such as a deoxyribonuclease, is used. In this case, the exonuclease enzyme is used to sequentially detach nucleotides from a nucleic acid (e.g. DNA) molecule. The nucleotides are then detected and discriminated by the nanopore in order of their release, thus reading the sequence of the original strand. For such an embodiment, the exonuclease enzyme can be attached to the nanopore such that a proportion of the nucleotides released from the DNA molecule is capable of entering and interacting with the channel of the nanopore. The exonuclease can be attached to the nanopore structure at a site in close proximity to the part of the nanopore that forms the opening of the channel. In some embodiments, the exonuclease enzyme can be attached to the nanopore structure such that its nucleotide exit trajectory site is orientated towards the part of the nanopore that forms part of the opening.

In some embodiments, nanopore sequencing of nucleic acids involves the use of an enzyme that pushes or pulls the nucleic acid (e.g. DNA) molecule through the pore. In this case, the ionic current fluctuates as a nucleotide in the DNA molecule passes through the pore. The fluctuations in the current are indicative of the DNA sequence. For such an embodiment, the enzyme can be attached to the nanopore structure such that it is capable of pushing or pulling a nucleic acid through the channel of a nanopore without interfering with the flow of ionic current through the pore. The enzyme can be attached to the nanopore structure at a site in close proximity to the part of the structure that forms part of the opening. The enzyme can be attached to the subunit, for example, such that its active site is orientated towards the part of the structure that forms part of the opening.

In some embodiments, nanopore sequencing of nucleic acids involves detection of polymerase bi-products in close proximity to a nanopore detector. In this case, nucleoside phosphates (nucleotides) are labeled so that a phosphate labeled species is released upon the addition of a polymerase to the nucleotide strand and the phosphate labeled species is detected by the pore. Typically, the phosphate species contains a specific label for each nucleotide. As nucleotides are sequentially added to the nucleic acid strand, the bi-products of the base addition are detected. The order that the phosphate labeled species are detected can be used to determine the sequence of the nucleic acid strand.

### Outcomes and Determination of the Presence or Absence of a Genetic Variation

Some genetic variations are associated with medical conditions. Genetic variations often include a gain, a loss, and/or alteration (e.g., reorganization or substitution) of genetic information (e.g., chromosomes, portions of chromosomes, polymorphic regions, translocated regions, altered nucleotide sequence, the like or combinations of the foregoing) that result in a detectable change in the genome or genetic information of a test subject with respect to a reference subject free of the genetic variation. The presence or absence of a genetic variation (e.g., fetal aneuploidy) can be determined by analyzing and/or manipulating nucleic acid quantification data (e.g. counts) as described herein.

Counting

In some embodiments, the amount of a targeted genomic region (e.g., chromosome) in a sample may be assessed based on the quantification of target fragments and/or reference fragments. In some embodiments, fragments obtained from a nucleic acid capture process are counted. A nucleic acid capture process, such as those described herein, may separate a subpopulation of nucleic acid fragments from the sample based on the genomic region (e.g., chromosome) from which the fragments originated. Thus, in some embodiments, fragments that correspond to a particular genomic region (e.g., chromosome) are counted. In some embodiments, fragments that correspond to a particular genomic region (e.g., chromosome) are counted and fragments that correspond to a different genomic region are not counted. In some embodiments, quantification of fragment species (e.g., target fragment species, reference fragment species, separated fragment species, separated target fragment species, separated reference fragment species) refers to counting of fragments that correspond to a particular genomic region (e.g., chromosome).

In some embodiments, fragments from a size fractionated sample are counted. In some embodiments, fragments of a certain length, range of lengths, or lengths under or over a particular threshold or cutoff are counted. In some embodiments, fragments of a certain length, range of lengths, or lengths under or over a particular threshold or cutoff are counted while fragments of a different length or range of length, or lengths over or under the threshold or cutoff are not counted. In some embodiments, quantification of fragment length species (e.g., target fragment length species, reference fragment length species, separated fragment length species, separated target fragment length species, separated reference fragment length species) refers to counting of fragments of a certain length, range of lengths, or lengths under or over a particular threshold or cutoff.

In some embodiments, fragments that are less than about 500 bp are counted. In some embodiments, fragments that are less than about 400 bp are counted. In some embodiments, fragments that are less than about 300 bp are counted. In some embodiments, fragments that are less than about 200 bp are counted. In some embodiments, fragments that are less than about 150 bp are counted. For example, fragments that are less than about 190 bp, 180 bp, 170 bp, 166 bp, 160 bp, 150 bp, 140 bp, 130 bp, 120 bp, 110 bp or 100 bp are counted. In some embodiments, fragments that are about 100 bp to about 200 bp are counted. For example, fragments that are about 190 bp, 180 bp, 170 bp, 160 bp, 150 bp, 145 bp, 140 bp, 135 bp, 130 bp, 120 bp or 110 bp are counted. In some embodiments, fragments that are in the range of about 100 bp to about 200 bp are counted. For example, fragments that are in the range of about 110 bp to about 190 bp, 130 bp to about 180 bp, 140 bp to about 170 bp, 140 bp to about 150 bp, 140 bp to about 160 bp, 150 bp to about 160 bp, 120 bp to about 150 bp, 120 bp to about 135 bp, 135 bp to about 150 bp, or 145 bp to about 155 bp are counted. In some embodiments, fragments that are 143 bp in length are counted. In some embodiments, fragments that are about 10 bp to about 30 bp shorter than other fragments of a certain length or range of lengths are counted. In some embodiments, fragments that are about 20 bp to about 25 bp shorter than other fragments of a certain length or range of lengths are counted. In some embodiments, fragments that are about 10 bp to about 20 bp shorter than other fragments of a certain length or range of lengths are counted. In some embodiments, fragments that are about 10 bp to about 15 bp shorter than other fragments of a certain length or range of lengths are counted. In some embodiments, fragments that have been counted may be referred to herein as “counts”, “data” or “data sets”.

In some embodiments, sequences of target fragments and/or reference fragments are obtained, as described herein. Such sequences may be aligned to a set of reference sequences, as described herein, and assigned to a particular genomic region (e.g., chromosome). Nucleotide sequences that have been assigned to a particular chromosome of interest, for example, can be quantified to determine the amount of corresponding genomic targets present in the sample, in some embodiments. In some embodiments, nucleotide sequences assigned to a reference chromosome also are counted.

Quantifying or counting fragments can be done in any suitable manner including but not limited to manual counting methods and automated counting methods. In some embodiments, an automated counting method can be embodied in software that determines or counts the number of nucleotide sequences and/or fragments assigned to each chromosome and/or one or more selected genomic regions. As used herein, software refers to computer readable program instructions that, when executed by a computer, perform computer operations.

In some embodiments, the number of counts assigned to one or more chromosomes of interest and/or a reference chromosome can be further analyzed and processed to provide an outcome determinative of the presence or absence of a genetic variation (e.g., fetal aneuploidy). In certain embodiments, counts can be organized into a matrix having two or more dimensions based on one or more features or variables. Data organized into matrices can be stratified using any suitable features or variables. A non-limiting example of data organized into a matrix includes data that is stratified by maternal age, maternal ploidy, and fetal contribution. In certain embodiments, data sets characterized by one or more features or variables sometimes are processed after counting. Examples of further analysis and processing of counts for fragments of a particular length or range of lengths, for example, is described in U.S. Patent Application Publication No. 2011/0276277, which is incorporated by reference in its entirety, and are described below.

Data Processing

Features (e.g. nucleotide sequences) that have been counted are sometimes referred to herein as raw data, since the data represent unmanipulated counts (e.g., raw counts). In some embodiments, data in a data set can be processed further (e.g., mathematically and/or statistically manipulated) and/or displayed to facilitate providing an outcome. In certain embodiments, data sets, including larger data sets, may benefit from pre-processing to facilitate further analysis. Pre-processing of data sets sometimes involves removal of redundant and/or uninformative data. Without being limited by theory, data processing and/or preprocessing may (i) remove noisy data, (ii) remove uninformative data, (iii) remove redundant data, (iv) reduce the complexity of larger data sets, and/or (v) facilitate transformation of the data from one form into one or more other forms. The terms “pre-processing” and “processing” when utilized with respect to data or data sets are collectively referred to herein as “processing”. Processing can render data more amenable to further analysis, and can generate an outcome in some embodiments.

The term “noisy data” as used herein refers to (a) data that has a significant variance between data points when analyzed or plotted, (b) data that has a significant standard deviation, (c) data that has a significant standard error of the mean, the like, and combinations of the foregoing. Noisy data sometimes occurs due to the quantity and/or quality of starting material (e.g., nucleic acid sample), and sometimes occurs as part of processes for preparing, replicating, separating, or amplifying DNA used to generate nucleotide sequence data or fragment counts, for example. In certain embodiments, noise results from certain nucleotide sequences being over represented when prepared using PCR-based methods. Methods described herein can reduce or eliminate the contribution of noisy data, and therefore reduce the effect of noisy data on the provided outcome.

Any suitable procedure can be utilized for processing data sets described herein. Non-limiting examples of procedures suitable for use for processing data sets include filtering, normalizing, weighting, monitoring peak heights, monitoring peak areas, monitoring peak edges, determining area ratios, mathematical processing of data, statistical processing of data, application of statistical algorithms, analysis with fixed variables, analysis with optimized variables, plotting data to identify patterns or trends for additional processing, the like and combinations of the foregoing. In certain embodiments, processing data sets as described herein can reduce the complexity and/or dimensionality of large and/or complex data sets. In some embodiments, data sets can include from hundreds to thousands to millions of data points for each test subject and/or test chromosome.

Data processing can be performed in any number of steps, in certain embodiments. For example, data may be processed using only a single processing procedure in some embodiments, and in certain embodiments data may be processed using 1 or more, 5 or more, 10 or more or 20 or more processing steps (e.g., 1 or more processing steps, 2 or more processing steps, 3 or more processing steps, 4 or more processing steps, 5 or more processing steps, 6 or more processing steps, 7 or more processing steps, 8 or more processing steps, 9 or more processing steps, 10 or more processing steps, 11 or more processing steps, 12 or more processing steps, 13 or more processing steps, 14 or more processing steps, 15 or more processing steps, 16 or more processing steps, 17 or more processing steps, 18 or more processing steps, 19 or more processing steps, or 20 or more processing steps). In some embodiments, processing steps may be the same step repeated two or more times (e.g., filtering two or more times, normalizing two or more times), and in certain embodiments, processing steps may be two or more different processing steps (e.g., filtering, normalizing; normalizing, monitoring peak heights and edges; filtering, normalizing, normalizing to a reference, statistical manipulation to determine p-values, and the like), carried out simultaneously or sequentially. In some embodiments, any suitable number and/or combination of the same or different processing steps can be utilized to process data to facilitate providing an outcome. In certain embodiments, processing data sets by the criteria described herein may reduce the complexity and/or dimensionality of a data set. In some embodiments, one or more processing steps can comprise one or more filtering steps.

In some embodiments, one or more processing steps can comprise one or more normalization steps. The term “normalization” as used herein refers to division of one or more data sets by a predetermined variable. Any suitable number of normalizations can be used. In some embodiments, data sets can be normalized 1 or more, 5 or more, 10 or more or even 20 or more times. Data sets can be normalized to values (e.g., normalizing value) representative of any suitable feature or variable (e.g., sample data, reference data, or both). Normalizing a data set sometimes has the effect of isolating statistical error, depending on the feature or property selected as the predetermined normalization variable. Normalizing a data set sometimes also allows comparison of data characteristics of data having different scales, by bringing the data to a common scale (e.g., predetermined normalization variable). In some embodiments, one or more normalizations to a statistically derived value can be utilized to minimize data differences and diminish the importance of outlying data.

In some embodiments, a processing step comprises a weighting. The terms “weighted”, “weighting” or “weight function” or grammatical derivatives or equivalents thereof, as used herein, refer to a mathematical manipulation of a portion or all of a data set sometimes utilized to alter the influence of certain data set features or variables with respect to other data set features or variables. A weighting function can be used to increase the influence of data with a relatively small measurement variance, and/or to decrease the influence of data with a relatively large measurement variance, in some embodiments. A non-limiting example of a weighting function is [1/(standard deviation)²]. A weighting step sometimes is performed in a manner substantially similar to a normalizing step. In some embodiments, a data set is divided by a predetermined variable (e.g., weighting variable). A predetermined variable (e.g., minimized target function, Phi) often is selected to weigh different parts of a data set differently (e.g., increase the influence of certain data types while decreasing the influence of other data types).

In certain embodiments, a processing step can comprise one or more mathematical and/or statistical manipulations. Any suitable mathematical and/or statistical manipulation, alone or in combination, may be used to analyze and/or manipulate a data set described herein. Any suitable number of mathematical and/or statistical manipulations can be used. In some embodiments, a data set can be mathematically and/or statistically manipulated 1 or more, 5 or more, 10 or more or 20 or more times. Non-limiting examples of mathematical and statistical manipulations that can be used include addition, subtraction, multiplication, division, algebraic functions, least squares estimators, curve fitting, differential equations, rational polynomials, double polynomials, orthogonal polynomials, z-scores, p-values, chi values, phi values, analysis of peak elevations, determination of peak edge locations, calculation of peak area ratios, analysis of median chromosomal elevation, calculation of mean absolute deviation, sum of squared residuals, mean, standard deviation, standard error, the like or combinations thereof. A mathematical and/or statistical manipulation can be performed on all or a portion of certain data, or processed products thereof. Non-limiting examples of data set variables or features that can be statistically manipulated include raw counts, filtered counts, normalized counts, peak heights, peak widths, peak areas, peak edges, lateral tolerances, P-values, median elevations, mean elevations, count distribution within a genomic region, relative representation of nucleic acid species, the like or combinations thereof.

In some embodiments, a processing step can include the use of one or more statistical algorithms. Any suitable statistical algorithm, alone or in combination, may be used to analyze and/or manipulate a data set described herein. Any suitable number of statistical algorithms can be used. In some embodiments, a data set can be analyzed using 1 or more, 5 or more, 10 or more or 20 or more statistical algorithms. Non-limiting examples of statistical algorithms suitable for use with methods described herein include decision trees, counternulls, multiple comparisons, omnibus test, Behrens-Fisher problem, bootstrapping, Fisher's method for combining independent tests of significance, null hypothesis, type I error, type II error, exact test, one-sample Z test, two-sample Z test, one-sample t-test, paired t-test, two-sample pooled t-test having equal variances, two-sample unpooled t-test having unequal variances, one-proportion z-test, two-proportion z-test pooled, two-proportion z-test unpooled, one-sample chi-square test, two-sample F test for equality of variances, confidence interval, credible interval, significance, meta analysis, simple linear regression, robust linear regression, the like or combinations of the foregoing. Non-limiting examples of data set variables or features that can be analyzed using statistical algorithms include raw counts, filtered counts, normalized counts, peak heights, peak widths, peak edges, lateral tolerances, P-values, median elevations, mean elevations, count distribution within a genomic region, relative representation of nucleic acid species, the like or combinations thereof.

In certain embodiments, a data set can be analyzed by utilizing multiple (e.g., 2 or more) statistical algorithms (e.g., least squares regression, principle component analysis, linear discriminant analysis, quadratic discriminant analysis, bagging, neural networks, support vector machine models, random forests, classification tree models, K-nearest neighbors, logistic regression and/or loss smoothing) and/or mathematical and/or statistical manipulations (e.g., referred to herein as manipulations). The use of multiple manipulations can generate an N-dimensional space that can be used to provide an outcome, in some embodiments. In certain embodiments, analysis of a data set by utilizing multiple manipulations can reduce the complexity and/or dimensionality of the data set. For example, the use of multiple manipulations on a reference data set can generate an N-dimensional space (e.g., probability plot) that can be used to represent the presence or absence of a genetic variation, depending on the genetic status of the reference samples (e.g., positive or negative for a selected genetic variation). Analysis of test samples using a substantially similar set of manipulations can be used to generate an N-dimensional point for each of the test samples. The complexity and/or dimensionality of a test subject data set sometimes is reduced to a single value or N-dimensional point that can be readily compared to the N-dimensional space generated from the reference data. Test sample data that fall within the N-dimensional space populated by the reference subject data are indicative of a genetic status substantially similar to that of the reference subjects. Test sample data that fall outside of the N-dimensional space populated by the reference subject data are indicative of a genetic status substantially dissimilar to that of the reference subjects. In some embodiments, references are euploid or do not otherwise have a genetic variation or medical condition. In some embodiments, references are presumed euploid (e.g., diploid) chromosomes, such as, for example, one or more of chromosomes 1, 2, 3, 4, 5, 6, 7, 8, 9, 10, 11, 12, 13, 14, 15, 16, 17, 18, 19, 20, 21, 22, X and/or Y.

In some embodiments, a processing step can comprise generating one or more profiles (e.g., profile plot) from various aspects of a data set or derivation thereof (e.g., product of one or more mathematical and/or statistical data processing steps known in the art and/or described herein). The term “profile” as used herein refers to mathematical and/or statistical manipulation of data that facilitates identification of patterns and/or correlations in large quantities of data. Thus, the term “profile” as used herein often refers to values resulting from one or more manipulations of data or data sets, based on one or more criteria. A profile often includes multiple data points. Any suitable number of data points may be included in a profile depending on the nature and/or complexity of a data set. In certain embodiments, profiles may include 2 or more data points, 3 or more data points, 5 or more data points, 10 or more data points, 24 or more data points, 25 or more data points, 50 or more data points, 100 or more data points, 500 or more data points, 1000 or more data points, 5000 or more data points, 10,000 or more data points, or 100,000 or more data points.

In some embodiments, a profile is representative of the entirety of a data set, and in certain embodiments, a profile is representative of a portion or subset of a data set. That is, a profile sometimes includes or is generated from data points representative of data that has not been filtered to remove any data, and sometimes a profile includes or is generated from data points representative of data that has been filtered to remove unwanted data. In some embodiments, a data point in a profile represents the results of data manipulation for a genomic region or chromosome. In certain embodiments, a data point in a profile represents the results of data manipulation for groups of genomic regions or chromosomes.

Data points in a profile derived from a data set can be representative of any suitable data categorization. In some embodiments, a profile may be generated from data points obtained from another profile (e.g., normalized data profile renormalized to a different normalizing value to generate a renormalized data profile). In certain embodiments, a profile generated from data points obtained from another profile reduces the number of data points and/or complexity of the data set. Reducing the number of data points and/or complexity of a data set often facilitates interpretation of data and/or facilitates providing an outcome.

A profile frequently is presented as a plot, and non-limiting examples of profile plots that can be generated include raw count (e.g., raw count profile or raw profile), normalized count (e.g., normalized count profile or normalized profile), z-score, p-value, area ratio versus fitted ploidy, median elevation versus ratio between fitted and measured fetal fraction, principle components, the like, or combinations thereof. Profile plots allow visualization of the manipulated data, in some embodiments. In certain embodiments, a profile plot can be utilized to provide an outcome (e.g., area ratio versus fitted ploidy, median elevation versus ratio between fitted and measured fetal fraction, principle components).

A profile generated for a test subject sometimes is compared to a profile generated for one or more reference subjects, to facilitate interpretation of mathematical and/or statistical manipulations of a data set and/or to provide an outcome. In some embodiments, a profile generated for a test chromosome is compared to profile generated for one or more reference chromosomes. In some embodiments, a reference chromosome is from the same individual as a test chromosome. In some embodiments, a reference chromosome and a test chromosome are from a different individuals. In some embodiments, a reference chromosome is the same as a test chromosome from another individual (e.g., chromosome 21 from a euploid individual versus chromosome 21 from an individual suspected or at risk of having an aneuploidy). In some embodiments, a reference chromosome and a test chromosome are different (e.g., chromosome 20 versus chromosome 21 from an individual suspected or at risk of having an aneuploidy). In some embodiments, a profile is generated based on one or more starting assumptions (e.g., maternal contribution of nucleic acid (e.g., maternal fraction), fetal contribution of nucleic acid (e.g., fetal fraction), ploidy of reference sample, the like or combinations thereof). In certain embodiments, a test profile often centers on a predetermined value representative of the absence of a genetic variation, and often deviates from a predetermined value in areas corresponding to the genomic location in which the genetic variation is located in the test subject, if the test subject possessed the genetic variation. In test subjects at risk for, or suffering from a medical condition associated with a genetic variation, the numerical value for a selected genomic region or chromosome is expected to vary significantly from the predetermined value for non-affected genomic locations. Depending on starting assumptions (e.g., fixed ploidy or optimized ploidy, fixed fetal fraction or optimized fetal fraction or combinations thereof) the predetermined threshold or cutoff value or range of values indicative of the presence or absence of a genetic variation can vary while still providing an outcome useful for determining the presence or absence of a genetic variation. In some embodiments, a profile is indicative of and/or representative of a phenotype.

In some embodiments, the use of one or more reference samples and/or chromosomes that are free of a genetic variation in question can be used to generate a reference median count profile, which may result in a predetermined value representative of the absence of the genetic variation, and often deviates from a predetermined value in areas corresponding to the genomic location in which the genetic variation is located in the test subject, if the test subject possessed the genetic variation. In test subjects at risk for, or suffering from a medical condition associated with a genetic variation, the numerical value for the selected genomic region or regions (e.g. chromosome or chromosomes) is expected to vary significantly from the predetermined value for non-affected genomic locations. In certain embodiments, the use of one or more reference samples known to carry the genetic variation in question can be used to generate a reference median count profile, which may result in a predetermined value representative of the presence of the genetic variation, and often deviates from a predetermined value in areas corresponding to the genomic location in which a test subject does not carry the genetic variation. In test subjects not at risk for, or suffering from a medical condition associated with a genetic variation, the numerical value for the selected genomic region is expected to vary significantly from the predetermined value for affected genomic locations.

In some embodiments, analysis and processing of data can include the use of one or more assumptions. Any suitable number or type of assumptions can be utilized to analyze or process a data set. Non-limiting examples of assumptions that can be used for data processing and/or analysis include maternal ploidy, fetal contribution, prevalence of certain nucleotide sequences and/or fragment species in a reference population, ethnic background, prevalence of a selected medical condition in related family members, parallelism between raw count profiles from different patients and/or runs after GC-normalization and repeat masking (e.g., GCRM), identical matches represent PCR artifacts (e.g., identical base position), assumptions inherent in a fetal quantifier assay (e.g., FQA), assumptions regarding twins (e.g., if 2 twins and only 1 is affected the effective fetal fraction is only 50% of the total measured fetal fraction (similarly for triplets, quadruplets and the like)), fetal cell free DNA (e.g., cfDNA) uniformly covers the entire genome, the like and combinations thereof.

In those instances where the quality and/or depth of data does not permit an outcome prediction of the presence or absence of a genetic variation at a desired confidence level (e.g., 95% or higher confidence level), based on the normalized count profiles, one or more additional mathematical manipulation algorithms and/or statistical prediction algorithms, can be utilized to generate additional numerical values useful for data analysis and/or providing an outcome. The term “normalized count profile” as used herein refers to a profile generated using normalized counts. Examples of methods that can be used to generate normalized counts and normalized count profiles are described herein. As noted, counts can be normalized with respect to test sample counts, reference sample counts, test chromosome counts and/or reference chromosome counts. In some embodiments, a normalized count profile can be presented as a plot.

As noted above, data sometimes is transformed from one form into another form. The terms “transformed”, “transformation”, and grammatical derivations or equivalents thereof, as used herein refer to an alteration of data from a physical starting material (e.g., test subject and/or reference subject sample nucleic acid; test chromosome and/or reference chromosome; target fragments and/or reference fragments) into a digital representation of the physical starting material, and in some embodiments includes a further transformation into one or more numerical values or graphical representations of the digital representation that can be utilized to provide an outcome. In certain embodiments, the one or more numerical values and/or graphical representations of digitally represented data can be utilized to represent the appearance of a test subject's physical genome (e.g., virtually represent or visually represent the presence or absence of a genomic insertion, genomic deletion and/or aneuploidy; represent the presence or absence of a variation in the physical amount of a nucleotide sequence, fragment, region or chromosome associated with medical conditions). A virtual representation sometimes is further transformed into one or more numerical values or graphical representations of the digital representation of the starting material. These procedures can transform physical starting material into a numerical value or graphical representation, or a representation of the physical appearance of a test subject's genome.

In some embodiments, transformation of a data set facilitates providing an outcome by reducing data complexity and/or data dimensionality. Data set complexity sometimes is reduced during the process of transforming a physical starting material into a virtual representation of the starting material. Any suitable feature or variable can be utilized to reduce data set complexity and/or dimensionality. Non-limiting examples of features that can be chosen for use as a target feature for data processing include GC content, fragment size (e.g., length), fragment sequence, fetal gender prediction, identification of chromosomal aneuploidy, identification of particular genes or proteins, identification of cancer, diseases, inherited genes/traits, chromosomal abnormalities, a biological category, a chemical category, a biochemical category, a category of genes or proteins, a gene ontology, a protein ontology, co-regulated genes, cell signaling genes, cell cycle genes, proteins pertaining to the foregoing genes, gene variants, protein variants, co-regulated genes, co-regulated proteins, amino acid sequence, nucleotide sequence, protein structure data and the like, and combinations of the foregoing. Non-limiting examples of data set complexity and/or dimensionality reduction include; reduction of a plurality of counts to profile plots, reduction of a plurality of counts to numerical values (e.g., normalized values, Z-scores, p-values); reduction of multiple analysis methods to probability plots or single points; principle component analysis of derived quantities; and the like or combinations thereof.

Outcome

Analysis and processing of data can provide one or more outcomes. The term “outcome” as used herein refers to a result of data processing that facilitates determining whether a subject was, or is, at risk of having a genetic variation. An outcome often comprises one or more numerical values generated using a processing method described herein in the context of one or more considerations of probability. A consideration of probability includes but is not limited to: measure of variability, confidence level, sensitivity, specificity, standard deviation, coefficient of variation (CV) and/or confidence level, Z-scores, Chi values, Phi values, ploidy values, fitted fetal fraction, area ratios, median elevation, the like or combinations thereof. A consideration of probability can facilitate determining whether a subject is at risk of having, or has, a genetic variation, and an outcome determinative of a presence or absence of a genetic disorder often includes such a consideration.

An outcome often is a phenotype with an associated level of confidence (e.g., fetus is positive for trisomy 21 with a confidence level of 99%, test subject is negative for a cancer associated with a genetic variation at a confidence level of 95%). Different methods of generating outcome values sometimes can produce different types of results. Generally, there are four types of possible scores or calls that can be made based on outcome values generated using methods described herein: true positive, false positive, true negative and false negative. The terms “score”, “scores”, “call” and “calls” as used herein refer to calculating the probability that a particular genetic variation is present or absent in a subject/sample. The value of a score may be used to determine, for example, a variation, difference, or ratio of counts that may correspond to a genetic variation. For example, calculating a positive score for a selected genetic variation or genomic region or chromosome from a data set, with respect to a reference genome and/or reference chromosome can lead to an identification of the presence or absence of a genetic variation, which genetic variation sometimes is associated with a medical condition (e.g., cancer, preeclampsia, trisomy, monosomy, and the like). In some embodiments, an outcome comprises a profile. In those embodiments in which an outcome comprises a profile, any suitable profile or combination of profiles can be used for an outcome. Non-limiting examples of profiles that can be used for an outcome include z-score profiles, p-value profiles, chi value profiles, phi value profiles, the like, and combinations thereof

An outcome generated for determining the presence or absence of a genetic variation sometimes includes a null result (e.g., a data point between two clusters, a numerical value with a standard deviation that encompasses values for both the presence and absence of a genetic variation, a data set with a profile plot that is not similar to profile plots for subjects having or free from the genetic variation being investigated). In some embodiments, an outcome indicative of a null result still is a determinative result, and the determination can include the need for additional information and/or a repeat of the data generation and/or analysis for determining the presence or absence of a genetic variation.

An outcome can be generated after performing one or more processing steps described herein, in some embodiments. In certain embodiments, an outcome is generated as a result of one of the processing steps described herein, and in some embodiments, an outcome can be generated after each statistical and/or mathematical manipulation of a data set is performed. An outcome pertaining to the determination of the presence or absence of a genetic variation can be expressed in any suitable form, which form comprises without limitation, a probability (e.g., odds ratio, p-value), likelihood, value in or out of a cluster, value over or under a threshold value, value with a measure of variance or confidence, or risk factor, associated with the presence or absence of a genetic variation for a subject or sample. In certain embodiments, comparison between samples allows confirmation of sample identity (e.g., allows identification of repeated samples and/or samples that have been mixed up (e.g., mislabeled, combined, and the like)).

In some embodiments, an outcome comprises a value above or below a predetermined threshold or cutoff value (e.g., greater than 1, less than 1), and an uncertainty or confidence level associated with the value. In some embodiments, a threshold can be set at about 1% or more elevation in counts (e.g., counts for a test chromosome versus a reference chromosome). For example, a threshold can be set at about 2%, 3%, 4%, 5%, 10%, 20%, 30%, 40%, 50% or more elevation in counts. An outcome also can describe any assumptions used in data processing. In certain embodiments, an outcome comprises a value that falls within or outside a predetermined range of values and the associated uncertainty or confidence level for that value being inside or outside the range. In some embodiments, an outcome comprises a value that is equal to a predetermined value (e.g., equal to 1, equal to zero), or is equal to a value within a predetermined value range, and its associated uncertainty or confidence level for that value being equal or within or outside a range. An outcome sometimes is graphically represented as a plot (e.g., profile plot).

As noted above, an outcome can be characterized as a true positive, true negative, false positive or false negative. The term “true positive” as used herein refers to a subject correctly diagnosed as having a genetic variation. The term “false positive” as used herein refers to a subject wrongly identified as having a genetic variation. The term “true negative” as used herein refers to a subject correctly identified as not having a genetic variation. The term “false negative” as used herein refers to a subject wrongly identified as not having a genetic variation. Two measures of performance for any given method can be calculated based on the ratios of these occurrences: (i) a sensitivity value, which generally is the fraction of predicted positives that are correctly identified as being positives; and (ii) a specificity value, which generally is the fraction of predicted negatives correctly identified as being negative. The term “sensitivity” as used herein refers to the number of true positives divided by the number of true positives plus the number of false negatives, where sensitivity (sens) may be within the range of 0≤sens≤1. Ideally, the number of false negatives equal zero or close to zero, so that no subject is wrongly identified as not having at least one genetic variation when they indeed have at least one genetic variation. Conversely, an assessment often is made of the ability of a prediction algorithm to classify negatives correctly, a complementary measurement to sensitivity. The term “specificity” as used herein refers to the number of true negatives divided by the number of true negatives plus the number of false positives, where sensitivity (spec) may be within the range of 0≤spec≤1. Ideally, the number of false positives equal zero or close to zero, so that no subject is wrongly identified as having at least one genetic variation when they do not have the genetic variation being assessed.

In certain embodiments, one or more of sensitivity, specificity and/or confidence level are expressed as a percentage. In some embodiments, the percentage, independently for each variable, is greater than about 90% (e.g., about 90, 91, 92, 93, 94, 95, 96, 97, 98 or 99%, or greater than 99% (e.g., about 99.5%, or greater, about 99.9% or greater, about 99.95% or greater, about 99.99% or greater)). Coefficient of variation (CV) in some embodiments is expressed as a percentage, and sometimes the percentage is about 10% or less (e.g., about 10, 9, 8, 7, 6, 5, 4, 3, 2 or 1%, or less than 1% (e.g., about 0.5% or less, about 0.1% or less, about 0.05% or less, about 0.01% or less)). A probability (e.g., that a particular outcome is not due to chance) in certain embodiments is expressed as a Z-score, a p-value, or the results of a t-test. In some embodiments, a measured variance, confidence interval, sensitivity, specificity and the like (e.g., referred to collectively as confidence parameters) for an outcome can be generated using one or more data processing manipulations described herein.

A method that has sensitivity and specificity equaling one, or 100%, or near one (e.g., between about 90% to about 99%) sometimes is selected. In some embodiments, a method having a sensitivity equaling 1, or 100% is selected, and in certain embodiments, a method having a sensitivity near 1 is selected (e.g., a sensitivity of about 90%, a sensitivity of about 91%, a sensitivity of about 92%, a sensitivity of about 93%, a sensitivity of about 94%, a sensitivity of about 95%, a sensitivity of about 96%, a sensitivity of about 97%, a sensitivity of about 98%, or a sensitivity of about 99%). In some embodiments, a method having a specificity equaling 1, or 100% is selected, and in certain embodiments, a method having a specificity near 1 is selected (e.g., a specificity of about 90%, a specificity of about 91%, a specificity of about 92%, a specificity of about 93%, a specificity of about 94%, a specificity of about 95%, a specificity of about 96%, a specificity of about 97%, a specificity of about 98%, or a specificity of about 99%).

After one or more outcomes have been generated, an outcome often is used to provide a determination of the presence or absence of a genetic variation and/or associated medical condition. An outcome typically is provided to a health care professional (e.g., laboratory technician or manager; physician or assistant). In some embodiments, an outcome determinative of the presence or absence of a genetic variation is provided to a healthcare professional in the form of a report, and in certain embodiments the report comprises a display of an outcome value and an associated confidence parameter. Generally, an outcome can be displayed in any suitable format that facilitates determination of the presence or absence of a genetic variation and/or medical condition. Non-limiting examples of formats suitable for use for reporting and/or displaying data sets or reporting an outcome include digital data, a graph, a 2D graph, a 3D graph, and 4D graph, a picture, a pictograph, a chart, a bar graph, a pie graph, a diagram, a flow chart, a scatter plot, a map, a histogram, a density chart, a function graph, a circuit diagram, a block diagram, a bubble map, a constellation diagram, a contour diagram, a cartogram, spider chart, Venn diagram, nomogram, and the like, and combination of the foregoing.

Use of Outcomes

A health care professional, or other qualified individual, receiving a report comprising one or more outcomes determinative of the presence or absence of a genetic variation can use the displayed data in the report to make a call regarding the status of the test subject or patient. The healthcare professional can make a recommendation based on the provided outcome, in some embodiments. A health care professional or qualified individual can provide a test subject or patient with a call or score with regards to the presence or absence of the genetic variation based on the outcome value or values and associated confidence parameters provided in a report, in some embodiments. In certain embodiments, a score or call is made manually by a healthcare professional or qualified individual, using visual observation of the provided report. In certain embodiments, a score or call is made by an automated routine, sometimes embedded in software, and reviewed by a healthcare professional or qualified individual for accuracy prior to providing information to a test subject or patient. The term “receiving a report” as used herein refers to obtaining, by any communication means, a written and/or graphical representation comprising an outcome, which upon review allows a healthcare professional or other qualified individual to make a determination as to the presence or absence of a genetic variation in a test subject or patient. The report may be generated by a computer or by human data entry, and can be communicated using electronic means (e.g., over the internet, via computer, via fax, from one network location to another location at the same or different physical sites), or by any other method of sending or receiving data (e.g., mail service, courier service and the like). In some embodiments the outcome is transmitted to a health care professional in a suitable medium, including, without limitation, in verbal, document, or file form. The file may be, for example, but not limited to, an auditory file, a computer readable file, a paper file, a laboratory file or a medical record file.

The term “providing an outcome” and grammatical equivalents thereof, as used herein also can refer to any method for obtaining outcome information, including, without limitation, obtaining the information from a laboratory file. A laboratory file can be generated by a laboratory that carried out one or more assays or one or more data processing steps to determine the presence or absence of the medical condition. The laboratory may be in the same location or different location (e.g., in another country) as the personnel identifying the presence or absence of the medical condition from the laboratory file. For example, the laboratory file can be generated in one location and transmitted to another location in which the information therein will be transmitted to the pregnant female subject. The laboratory file may be in tangible form or electronic form (e.g., computer readable form), in certain embodiments.

A healthcare professional or qualified individual, can provide any suitable recommendation based on the outcome or outcomes provided in the report. Non-limiting examples of recommendations that can be provided based on the provided outcome report includes, surgery, radiation therapy, chemotherapy, genetic counseling, after birth treatment solutions (e.g., life planning, long term assisted care, medicaments, symptomatic treatments), pregnancy termination, organ transplant, blood transfusion, the like or combinations of the foregoing. In some embodiments the recommendation is dependent on the outcome based classification provided (e.g., Down's syndrome, Turner syndrome, medical conditions associated with genetic variations in T13, medical conditions associated with genetic variations in T18).

Software can be used to perform one or more steps in the process described herein, including but not limited to; counting, data processing, generating an outcome, and/or providing one or more recommendations based on generated outcomes.

### Machines, Software and Interfaces

Apparatuses, software and interfaces may be used to conduct methods described herein. Using apparatuses, software and interfaces, a user may enter, request, query or determine options for using particular information, programs or processes (e.g., selecting nucleotide sequences for designing a nucleic acid capture method, aligning nucleotide sequences, generating counts, processing data and/or providing an outcome), which can involve implementing statistical analysis algorithms, statistical significance algorithms, statistical algorithms, iterative steps, validation algorithms, and graphical representations, for example. In some embodiments, a data set may be entered by a user as input information, a user may download one or more data sets by any suitable hardware media (e.g., flash drive), and/or a user may send a data set from one system to another for subsequent processing and/or providing an outcome (e.g., send nucleotide sequence data from a sequencer to a computer system for nucleotide sequence alignment; send aligned nucleotide sequence data to a computer system for processing and yielding an outcome and/or report).

A user may, for example, place a query to software which then may acquire a data set via internet access, and in certain embodiments, a programmable processor may be prompted to acquire a suitable data set based on given parameters. A programmable processor also may prompt a user to select one or more data set options selected by the processor based on given parameters. A programmable processor may prompt a user to select one or more data set options selected by the processor based on information found via the internet, other internal or external information, or the like. Options may be chosen for selecting one or more data feature selections, one or more statistical algorithms, one or more statistical analysis algorithms, one or more statistical significance algorithms, iterative steps, one or more validation algorithms, and one or more graphical representations of methods, apparatuses, or computer programs.

Systems addressed herein may comprise general components of computer systems, such as, for example, network servers, laptop systems, desktop systems, handheld systems, personal digital assistants, computing kiosks, and the like. A computer system may comprise one or more input means such as a keyboard, touch screen, mouse, voice recognition or other means to allow the user to enter data into the system. A system may further comprise one or more outputs, including, but not limited to, a display screen (e.g., CRT or LCD), speaker, FAX machine, printer (e.g., laser, ink jet, impact, black and white or color printer), or other output useful for providing visual, auditory and/or hardcopy output of information (e.g., outcome and/or report).

In a system, input and output means may be connected to a central processing unit which may comprise among other components, a microprocessor for executing program instructions and memory for storing program code and data. In some embodiments, processes may be implemented as a single user system located in a single geographical site. In certain embodiments, processes may be implemented as a multi-user system. In the case of a multi-user implementation, multiple central processing units may be connected by means of a network. The network may be local, encompassing a single department in one portion of a building, an entire building, span multiple buildings, span a region, span an entire country or be worldwide. The network may be private, being owned and controlled by a provider, or it may be implemented as an internet based service where the user accesses a web page to enter and retrieve information. Accordingly, in certain embodiments, a system includes one or more machines, which may be local or remote with respect to a user. More than one machine in one location or multiple locations may be accessed by a user, and data may be obtained and/or processed in series and/or in parallel. Thus, any suitable configuration and control may be utilized for obtaining and/or processing data using multiple machines, such as in local network, remote network and/or “cloud” computing platforms.

A system can include a communications interface in some embodiments. A communications interface allows for transfer of software and data between a computer system and one or more external devices. Non-limiting examples of communications interfaces include a modem, a network interface (such as an Ethernet card), a communications port, a PCMCIA slot and card, and the like. Software and data transferred via a communications interface generally are in the form of signals, which can be electronic, electromagnetic, optical and/or other signals capable of being received by a communications interface. Signals often are provided to a communications interface via a channel. A channel often carries signals and can be implemented using wire or cable, fiber optics, a phone line, a cellular phone link, an RF link and/or other communications channels. Thus, in an example, a communications interface may be used to receive signal information that can be detected by a signal detection module.

Data may be input by any suitable device and/or method, including, but not limited to, manual input devices or direct data entry devices (DDEs). Non-limiting examples of manual devices include keyboards, concept keyboards, touch sensitive screens, light pens, mouse, tracker balls, joysticks, graphic tablets, scanners, digital cameras, video digitizers and voice recognition devices. Non-limiting examples of DDEs include bar code readers, magnetic strip codes, smart cards, magnetic ink character recognition, optical character recognition, optical mark recognition, and turnaround documents.

In some embodiments, output from a sequencing apparatus may serve as data that can be input via an input device. In certain embodiments, aligned nucleotide sequences may serve as data that can be input via an input device. In certain embodiments, nucleic acid fragment size (e.g., length) may serve as data that can be input via an input device. In certain embodiments, output from a nucleic acid capture process (e.g., genomic region origin data) may serve as data that can be input via an input device. In certain embodiments, a combination of nucleic acid fragment size (e.g., length) and output from a nucleic acid capture process (e.g., genomic region origin data) may serve as data that can be input via an input device. In certain embodiments, simulated data is generated by an in silico process and the simulated data serves as data that can be input via an input device. The term “in silico” refers to research and experiments performed using a computer. In silico processes include, but are not limited to, aligning nucleotide sequences and processing aligned nucleotide sequences according to processes described herein.

A system may include software useful for performing a process described herein, and software can include one or more modules for performing such processes (e.g., data acquisition module, data processing module, data display module). The term “software” refers to computer readable program instructions that, when executed by a computer, perform computer operations. The term “module” refers to a self-contained functional unit that can be used in a larger software system. For example, a software module is a part of a program that performs a particular process or task.

Software often is provided on a program product containing program instructions recorded on a computer readable medium, including, but not limited to, magnetic media including floppy disks, hard disks, and magnetic tape; and optical media including CD-ROM discs, DVD discs, magneto-optical discs, flash drives, RAM, floppy discs, the like, and other such media on which the program instructions can be recorded. In online implementation, a server and web site maintained by an organization can be configured to provide software downloads to remote users, or remote users may access a remote system maintained by an organization to remotely access software.

Software may obtain or receive input information. Software may include a module that specifically obtains or receives data (e.g., a data receiving module that receives nucleotide sequence data and/or aligned nucleotide sequence data) and may include a module that specifically processes the data (e.g., a processing module that processes received data (e.g., filters, normalizes, provides an outcome and/or report). The terms “obtaining” and “receiving” input information refers to receiving data (e.g., nucleotide sequences, aligned nucleotide sequences) by computer communication means from a local, or remote site, human data entry, or any other method of receiving data. The input information may be generated in the same location at which it is received, or it may be generated in a different location and transmitted to the receiving location. In some embodiments, input information is modified before it is processed (e.g., placed into a format amenable to processing (e.g., tabulated)).

Software can include one or more algorithms in certain embodiments. An algorithm may be used for processing data and/or providing an outcome or report according to a finite sequence of instructions. An algorithm often is a list of defined instructions for completing a task. Starting from an initial state, the instructions may describe a computation that proceeds through a defined series of successive states, eventually terminating in a final ending state. The transition from one state to the next is not necessarily deterministic (e.g., some algorithms incorporate randomness). By way of example, and without limitation, an algorithm can be a search algorithm, sorting algorithm, merge algorithm, numerical algorithm, graph algorithm, string algorithm, modeling algorithm, computational geometric algorithm, combinatorial algorithm, machine learning algorithm, cryptography algorithm, data compression algorithm, parsing algorithm and the like. An algorithm can include one algorithm or two or more algorithms working in combination. An algorithm can be of any suitable complexity class and/or parameterized complexity. An algorithm can be used for calculation and/or data processing, and in some embodiments, can be used in a deterministic or probabilistic/predictive approach. An algorithm can be implemented in a computing environment by use of a suitable programming language, non-limiting examples of which are C, C++, Java, Perl, Python, Fortran, and the like. In some embodiments, an algorithm can be configured or modified to include margin of errors, statistical analysis, statistical significance, and/or comparison to other information or data sets (e.g., applicable when using a neural net or clustering algorithm).

In certain embodiments, several algorithms may be implemented for use in software. These algorithms can be trained with raw data in some embodiments. For each new raw data sample, the trained algorithms may produce a representative processed data set or outcome. A processed data set sometimes is of reduced complexity compared to the parent data set that was processed. Based on a processed set, the performance of a trained algorithm may be assessed based on sensitivity and specificity, in some embodiments. An algorithm with the highest sensitivity and/or specificity may be identified and utilized, in certain embodiments.

In certain embodiments, simulated (or simulation) data can aid data processing, for example, by training an algorithm or testing an algorithm. In some embodiments, simulated data includes hypothetical various samplings of different groupings of data or counts. Simulated data may be based on what might be expected from a real population or may be skewed to test an algorithm and/or to assign a correct classification. Simulated data also is referred to herein as “virtual” data. Simulations can be performed by a computer program in certain embodiments. One possible step in using a simulated data set is to evaluate the confidence of an identified result, e.g., how well a random sampling matches or best represents the original data. One approach is to calculate a probability value (p-value), which estimates the probability of a random sample having better score than the selected samples. In some embodiments, an empirical model may be assessed, in which it is assumed that at least one sample matches a reference sample (with or without resolved variations). In some embodiments, another distribution, such as a Poisson distribution for example, can be used to define the probability distribution.

A system may include one or more processors in certain embodiments. A processor can be connected to a communication bus. A computer system may include a main memory, often random access memory (RAM), and can also include a secondary memory. Secondary memory can include, for example, a hard disk drive and/or a removable storage drive, representing a floppy disk drive, a magnetic tape drive, an optical disk drive, memory card and the like. A removable storage drive often reads from and/or writes to a removable storage unit. Non-limiting examples of removable storage units include a floppy disk, magnetic tape, optical disk, and the like, which can be read by and written to by, for example, a removable storage drive. A removable storage unit can include a computer-usable storage medium having stored therein computer software and/or data.

A processor may implement software in a system. In some embodiments, a processor may be programmed to automatically perform a task described herein that a user could perform. Accordingly, a processor, or algorithm conducted by such a processor, can require little to no supervision or input from a user (e.g., software may be programmed to implement a function automatically). In some embodiments, the complexity of a process is so large that a single person or group of persons could not perform the process in a timeframe short enough for providing an outcome determinative of the presence or absence of a genetic variation.

In some embodiments, secondary memory may include other similar means for allowing computer programs or other instructions for loading into a computer system. For example, a system can include a removable storage unit and an interface device. Non-limiting examples of such systems include a program cartridge and cartridge interface (such as that found in video game devices), a removable memory chip (such as an EPROM, or PROM) and associated socket, and other removable storage units and interfaces that allow software and data for transfer from the removable storage unit to a computer system.

### Genetic Variations and Medical Conditions

The presence or absence of a genetic variance can be determined using a method or apparatus described herein. In certain embodiments, the presence of absence of one or more genetic variations is determined according to an outcome provided by methods and apparatuses described herein. A genetic variation generally is a particular genetic phenotype present in certain individuals, and often a genetic variation is present in a statistically significant sub-population of individuals. Non-limiting examples of genetic variations include one or more deletions (e.g., micro-deletions), insertions, mutations, polymorphisms (e.g., single-nucleotide polymorphisms), fusions, repeats (e.g., short tandem repeats), distinct methylation sites, distinct methylation patterns, the like and combinations thereof. An insertion, repeat, deletion, mutation or polymorphism can be of any observed length, and in some embodiments, is about 1 base or base pair (bp) to 1,000 kilobases (kb) in length (e.g., about 10 bp, 50 bp, 100 bp, 500 bp, 1 kb, 5 kb, 10 kb, 50 kb, 100 kb or 500 kb in length). In some embodiments, a genetic variation is a chromosome abnormality (e.g., aneuploidy), partial chromosome abnormality or mosaicism, each of which is described in greater detail hereafter.

A genetic variation for which the presence or absence is identified for a subject is associated with a medical condition in certain embodiments. Thus, technology described herein can be used to identify the presence or absence of one or more genetic variations that are associated with a medical condition or medical state. Non-limiting examples of medical conditions include those associated with intellectual disability (e.g., Down Syndrome), aberrant cell-proliferation (e.g., cancer), presence of a micro-organism nucleic acid (e.g., virus, bacterium, fungus, yeast), and preeclampsia.

Non-limiting examples of genetic variations, medical conditions and states are described hereafter.

Fetal Gender

In some embodiments, the prediction of a fetal gender can be determined by a method or apparatus described herein. Gender determination generally is based on a sex chromosome. In humans, there are two sex chromosomes, the X and Y chromosomes. Individuals with XX are female and XY are male and non-limiting variations include XO, XYY, XXX and XXY.

Chromosome Abnormalities

In some embodiments, the presence or absence of a fetal chromosome abnormality can be determined by using a method or apparatus described herein. Chromosome abnormalities include, without limitation, a gain or loss of an entire chromosome or a region of a chromosome comprising one or more genes. Chromosome abnormalities include monosomies, trisomies, polysomies, loss of heterozygosity, deletions and/or duplications of one or more nucleotide sequences (e.g., one or more genes), including deletions and duplications caused by unbalanced translocations. The terms “aneuploidy” and “aneuploid” as used herein refer to an abnormal number of chromosomes in cells of an organism. As different organisms have widely varying chromosome complements, the term “aneuploidy” does not refer to a particular number of chromosomes, but rather to the situation in which the chromosome content within a given cell or cells of an organism is abnormal.

The term “monosomy” as used herein refers to lack of one chromosome of the normal complement. Partial monosomy can occur in unbalanced translocations or deletions, in which only a portion of the chromosome is present in a single copy. Monosomy of sex chromosomes (45, X) causes Turner syndrome, for example.

The term “disomy” refers to the presence of two copies of a chromosome. For organisms such as humans that have two copies of each chromosome (those that are diploid or “euploid”), disomy is the normal condition. For organisms that normally have three or more copies of each chromosome (those that are triploid or above), disomy is an aneuploid chromosome state. In uniparental disomy, both copies of a chromosome come from the same parent (with no contribution from the other parent).

The term “trisomy” as used herein refers to the presence of three copies, instead of two copies, of a particular chromosome. The presence of an extra chromosome 21, which is found in human Down syndrome, is referred to as “Trisomy 21.” Trisomy 18 and Trisomy 13 are two other human autosomal trisomies. Trisomy of sex chromosomes can be seen in females (e.g., 47, XXX) or males (e.g., 47, XXY in Klinefelter's syndrome; or 47,XYY).

The terms “tetrasomy” and “pentasomy” as used herein refer to the presence of four or five copies of a chromosome, respectively. Although rarely seen with autosomes, sex chromosome tetrasomy and pentasomy have been reported in humans, including XXXX, XXXY, XXYY, XYYY, XXXXX, XXXXY, XXXYY, XXYYY and XYYYY.

Chromosome abnormalities can be caused by a variety of mechanisms. Mechanisms include, but are not limited to (i) nondisjunction occurring as the result of a weakened mitotic checkpoint, (ii) inactive mitotic checkpoints causing non-disjunction at multiple chromosomes, (iii) merotelic attachment occurring when one kinetochore is attached to both mitotic spindle poles, (iv) a multipolar spindle forming when more than two spindle poles form, (v) a monopolar spindle forming when only a single spindle pole forms, and (vi) a tetraploid intermediate occurring as an end result of the monopolar spindle mechanism.

The terms “partial monosomy” and “partial trisomy” as used herein refer to an imbalance of genetic material caused by loss or gain of part of a chromosome. A partial monosomy or partial trisomy can result from an unbalanced translocation, where an individual carries a derivative chromosome formed through the breakage and fusion of two different chromosomes. In this situation, the individual would have three copies of part of one chromosome (two normal copies and the portion that exists on the derivative chromosome) and only one copy of part of the other chromosome involved in the derivative chromosome.

The term “mosaicism” as used herein refers to aneuploidy in some cells, but not all cells, of an organism. Certain chromosome abnormalities can exist as mosaic and non-mosaic chromosome abnormalities. For example, certain trisomy 21 individuals have mosaic Down syndrome and some have non-mosaic Down syndrome. Different mechanisms can lead to mosaicism. For example, (i) an initial zygote may have three 21st chromosomes, which normally would result in simple trisomy 21, but during the course of cell division one or more cell lines lost one of the 21st chromosomes; and (ii) an initial zygote may have two 21st chromosomes, but during the course of cell division one of the 21st chromosomes were duplicated. Somatic mosaicism likely occurs through mechanisms distinct from those typically associated with genetic syndromes involving complete or mosaic aneuploidy. Somatic mosaicism has been identified in certain types of cancers and in neurons, for example. In certain instances, trisomy 12 has been identified in chronic lymphocytic leukemia (CLL) and trisomy 8 has been identified in acute myeloid leukemia (AML). Also, genetic syndromes in which an individual is predisposed to breakage of chromosomes (chromosome instability syndromes) are frequently associated with increased risk for various types of cancer, thus highlighting the role of somatic aneuploidy in carcinogenesis. Methods and protocols described herein can identify presence or absence of non-mosaic and mosaic chromosome abnormalities.

Following is a non-limiting list of chromosome abnormalities that can be potentially identified by methods and apparatus described herein.

| Chromosome | Abnormality | Disease Association |
|---|---|---|
| X | XO | Turner's Syndrome |
| Y | XXY | Klinefelter syndrome |
| Y | XYY | Double Y syndrome |
| Y | XXX | Trisomy X syndrome |
| Y | XXXX | Four X syndrome |
| Y | Xp21 deletion | Duchenne's/Becker syndrome, congenital adrenal |
| | | hypoplasia, chronic granulomatus disease |
| Y | Xp22 deletion | steroid sulfatase deficiency |
| Y | Xq26 deletion | X-linked lymphproliferative disease |
| 1 | 1p (somatic) | neuroblastoma |
| | monosomy | |
| | trisomy | |
| 2 | monosomy | growth retardation, developmental and mental delay, |
| | trisomy 2q | and minor physical abnormalities |
| 3 | monosomy | Non-Hodgkin's lymphoma |
| | trisomy (somatic) | |
| 4 | monosomy | Acute non lymphocytic leukemia (ANLL) |
| | trisomy (somatic) | |
| 5 | 5p | Cri du chat; Lejeune syndrome |
| 5 | 5q | myelodysplastic syndrome |
| | (somatic) | |
| | monosomy | |
| | trisomy | |
| 6 | monosomy | clear-cell sarcoma |
| | trisomy (somatic) | |
| 7 | 7q11.23 deletion | William's syndrome |
| 7 | monosomy | monosomy 7 syndrome of childhood; somatic: renal |
| | trisomy | cortical adenomas; myelodysplastic syndrome |
| 8 | 8q24.1 deletion | Langer-Giedon syndrome |
| 8 | monosomy | myelodysplastic syndrome; Warkany syndrome; |
| | trisomy | somatic: chronic myelogenous leukemia |
| 9 | monosomy 9p | Alfi's syndrome |
| 9 | monosomy 9p | Rethore syndrome |
| | partial trisomy | |
| 9 | trisomy | complete trisomy 9 syndrome; mosaic trisomy 9 |
| | | syndrome |
| 10 | Monosomy | ALL or ANLL |
| | trisomy (somatic) | |
| 11 | 11p- | Aniridia; Wilms tumor |
| 11 | 11q- | Jacobson Syndrome |
| 11 | monosomy | myeloid lineages affected (ANLL, MDS) |
| | (somatic) trisomy | |
| 12 | monosomy | CLL, Juvenile granulosa cell tumor (JGCT) |
| | trisomy (somatic) | |
| 13 | 13q- | 13q-syndrome; Orbeli syndrome |
| 13 | 13q14 deletion | retinoblastoma |
| 13 | monosomy | Patau's syndrome |
| | trisomy | |
| 14 | monosomy | myeloid disorders (MDS, ANLL, atypical CML) |
| | trisomy (somatic) | |
| 15 | 15q11-q13 | Prader-Willi, Angelman's syndrome |
| | deletion | |
| | monosomy | |
| 15 | trisomy (somatic) | myeloid and lymphoid lineages affected, e.g., MDS, |
| | | ANLL, ALL, CLL) |
| 16 | 16q13.3 deletion | Rubenstein-Taybi |
| | monosomy | papillary renal cell carcinomas (malignant) |
| | trisomy (somatic) | |
| 17 | 17p-(somatic) | 17p syndrome in myeloid malignancies |
| 17 | 17q11.2 deletion | Smith-Magenis |
| 17 | 17q13.3 | Miller-Dieker |
| 17 | monosomy | renal cortical adenomas |
| | trisomy (somatic) | |
| 17 | 17p11.2-12 | Charcot-Marie Tooth Syndrome type 1; HNPP |
| | trisomy | |
| 18 | 18p- | 18p partial monosomy syndrome or Grouchy Lamy |
| | | Thieffry syndrome |
| 18 | 18q- | Grouchy Lamy Salmon Landry Syndrome |
| 18 | monosomy | Edwards Syndrome |
| | trisomy | |
| 19 | monosomy | |
| | trisomy | |
| 20 | 20p- | trisomy 20p syndrome |
| 20 | 20p11.2-12 | Alagille |
| | deletion | |
| 20 | 20q- | somatic: MDS, ANLL, polycythemia vera, chronic |
| | | neutrophilic leukemia |
| 20 | monosomy | papillary renal cell carcinomas (malignant) |
| | trisomy (somatic) | |
| 21 | monosomy | Down's syndrome |
| | trisomy | |
| 22 | 22q11.2 deletion | DiGeorge's syndrome, velocardiofacial syndrome, |
| | | conotruncal anomaly face syndrome, autosomal |
| | | dominant Opitz G/BBB syndrome, Caylor cardiofacial |
| | | syndrome |
| 22 | monosomy | complete trisomy 22 syndrome |
| | trisomy | |

Preeclampsia

In some embodiments, the presence or absence of preeclampsia is determined by using a method or apparatus described herein. Preeclampsia is a condition in which hypertension arises in pregnancy (i.e. pregnancy-induced hypertension) and is associated with significant amounts of protein in the urine. In some embodiments, preeclampsia also is associated with elevated levels of extracellular nucleic acid and/or alterations in methylation patterns. For example, a positive correlation between extracellular fetal-derived hypermethylated RASSF1A levels and the severity of preeclampsia has been observed. In certain examples, increased DNA methylation is observed for the H19 gene in preeclamptic placentas compared to normal controls.

Preeclampsia is one of the leading causes of maternal and fetal/neonatal mortality and morbidity worldwide. Circulating cell-free nucleic acids in plasma and serum are novel biomarkers with promising clinical applications in different medical fields, including prenatal diagnosis. Quantitative changes of cell-free fetal (cff)DNA in maternal plasma as an indicator for impending preeclampsia have been reported in different studies, for example, using real-time quantitative PCR for the male-specific SRY or DYS 14 loci. In cases of early onset preeclampsia, elevated levels may be seen in the first trimester. The increased levels of cffDNA before the onset of symptoms may be due to hypoxia/reoxygenation within the intervillous space leading to tissue oxidative stress and increased placental apoptosis and necrosis. In addition to the evidence for increased shedding of cffDNA into the maternal circulation, there is also evidence for reduced renal clearance of cffDNA in preeclampsia. As the amount of fetal DNA is currently determined by quantifying Y-chromosome specific sequences, other approaches such as measurement of total cell-free DNA or the use of gender-independent fetal epigenetic markers, such as DNA methylation, offer an alternative. Cell-free RNA of placental origin is another alternative biomarker that may be used for screening and diagnosing preeclampsia in clinical practice. Fetal RNA is associated with subcellular placental particles that protect it from degradation. Fetal RNA levels sometimes are ten-fold higher in pregnant females with preeclampsia compared to controls, and therefore is an alternative biomarker that may be used for screening and diagnosing preeclampsia in clinical practice.

Pathogens

In some embodiments, the presence or absence of a pathogenic condition is determined by a method or apparatus described herein. A pathogenic condition can be caused by infection of a host by a pathogen including, but not limited to, a bacterium, virus or fungus. Since pathogens typically possess nucleic acid (e.g., genomic DNA, genomic RNA, mRNA) that can be distinguishable from host nucleic acid, methods and apparatus provided herein can be used to determine the presence or absence of a pathogen. Often, pathogens possess nucleic acid with characteristics unique to a particular pathogen such as, for example, epigenetic state and/or one or more sequence variations, duplications and/or deletions. Thus, methods provided herein may be used to identify a particular pathogen or pathogen variant (e.g. strain).

Cancers

In some embodiments, the presence or absence of a cell proliferation disorder (e.g., a cancer) is determined by using a method or apparatus described herein. For example, levels of cell-free nucleic acid in serum can be elevated in patients with various types of cancer compared with healthy patients. Patients with metastatic diseases, for example, can sometimes have serum DNA levels approximately twice as high as non-metastatic patients. Patients with metastatic diseases may also be identified by cancer-specific markers and/or certain single nucleotide polymorphisms or short tandem repeats, for example. Non-limiting examples of cancer types that may be positively correlated with elevated levels of circulating DNA include breast cancer, colorectal cancer, gastrointestinal cancer, hepatocellular cancer, lung cancer, melanoma, non-Hodgkin lymphoma, leukemia, multiple myeloma, bladder cancer, hepatoma, cervical cancer, esophageal cancer, pancreatic cancer, and prostate cancer. Various cancers can possess, and can sometimes release into the bloodstream, nucleic acids with characteristics that are distinguishable from nucleic acids from non-cancerous healthy cells, such as, for example, epigenetic state and/or sequence variations, duplications and/or deletions. Such characteristics can, for example, be specific to a particular type of cancer. Thus, it is further contemplated that the methods provided herein can be used to identify a particular type of cancer.

### EXAMPLES

The examples set forth below illustrate certain embodiments and do not limit the technology.

### Example 1: Mass-Modified Nucleotide Design

Four sets of mass-modified nucleotides (set A (A1 and A2), set B (B1 and B2), set C (C1 and C2), and set D (D1 and D2) are described in this example. Molar masses provided below refer to the molar mass of each base (i.e., nucleotide base without the sugar or phosphate(s)). Changes in molar mass for each base may be applied to the overall molar mass change for the corresponding nucleotide (i.e., base plus sugar and phosphate(s)). For ease of numerical comparison, mass changes resulting from modifications to the sugar or phosphate are shown as a net change in the molar mass of the base.

Set A

A first set (set A1, set A2) of mass-modified nucleotides are designed according to the following scheme. In sets A1 and A2, each of adenine (A), thymine (T), guanine (G), and cytosine (C) are mass-modified such that their masses are substantially identical. The thymine base has the molecular formula C5H6N2O2 and molar mass of about 126 g/mol. An azide (N3) group is added to thymine, which increases the molar mass by about 41 AMU to about 167 g/mol. Guanine has the molecular formula C5H5N5O and molar mass of about 151 g/mol, which is about 16 AMU less than the modified thymine nucleotide above. To increase the molar mass of guanine by about 16 AMU, one hydrogen atom is replaced with one methyl group having one carbon atom, one hydrogen atom and two deuterium atoms (net gain of about 16 AMU). Adenine has the molecular formula C5H5N5 and molar mass of about 135 g/mol, which is about 32 AMU less than the modified nucleotides above. To increase the molar mass of adenine by about 32 AMU, one hydrogen atom is replaced with one sulfhydryl group having one sulfur atom and one hydrogen atom (net gain of about 32 AMU). Cytosine has the molecular formula C4H5N3O and molar mass of about 111 g/mol, which is about 56 AMU less than the modified nucleotides above. To increase the molar mass of cytosine by about 56 AMU, a hydrogen atom is replaced with a methyl azide group having one carbon 13 isotope, two hydrogen atoms, and three nitrogen atoms (net gain of about 56 AMU).

The mass modifications for the nucleotides of set A1 are in the purine base (at position C-8) or pyrimidine base (at position C-5), as shown in the structures below.

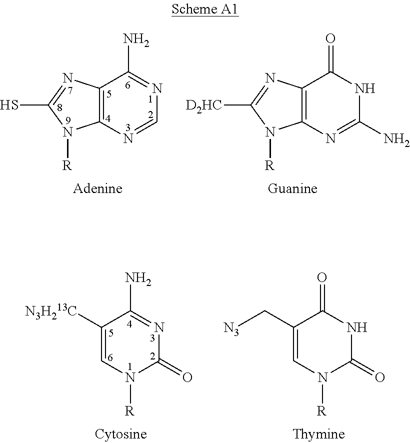

The mass modifications for the nucleotides of set A2 are at position C2′ in the sugar of the corresponding nucleosides, as shown in the structures below.

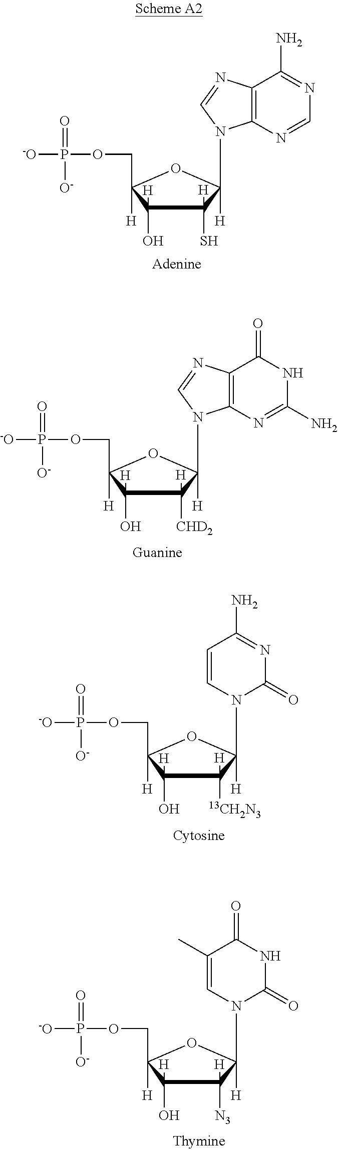

Set B

A second set (set B1, set B2) of mass-modified nucleotides are designed according to the following scheme. Each of adenine (A), thymine (T), and cytosine (C) are mass-modified such that their masses are substantially identical to guanine (G), which is has a molar mass of about 151 g/mol. Adenine has the molecular formula C5H5N5 and molar mass of about 135 g/mol, which is about 16 AMU less than guanine. To increase the molar mass of adenine by about 16 AMU, one hydrogen atom is replaced with one methyl group having one carbon atom, one hydrogen atom and two deuterium atoms (net gain of about 16 AMU). Thymine has the molecular formula C5H6N2O2 and molar mass of about 126 g/mol, which is about 25 AMU less than guanine. To increase the molar mass of thymine by about 25 AMU, 1) one oxygen atom is replaced with one sulfur atom (net gain of about 16 AMU); 2) five hydrogen atoms are replaced with five deuterium atoms (net gain of about 5 AMU); and 3) four carbon atoms are replaced with four carbon 13 isotopes (net gain of about 4 AMU). Cytosine has the molecular formula C4H5N3O and molar mass of about 111 g/mol, which is about 40 AMU less than guanine. To increase the molar mass of cytosine by about 40 AMU, 1) one oxygen atom is replaced with one sulfur atom (net gain of about 16 AMU); 2) three hydrogen atoms are replaced with three deuterium atoms (net gain of about 3 AMU); 3) four carbon atoms are replaced with four carbon 13 isotopes (net gain of about 4 AMU); and 4) one hydrogen atom is replaced with one methyl group having one carbon atom and three deuterium atoms (net gain of about 17 AMU).

The mass modifications for the nucleotides of set B1 are in the purine base (at position C-8) or pyrimidine base (at position C-5), as shown in the structures below.

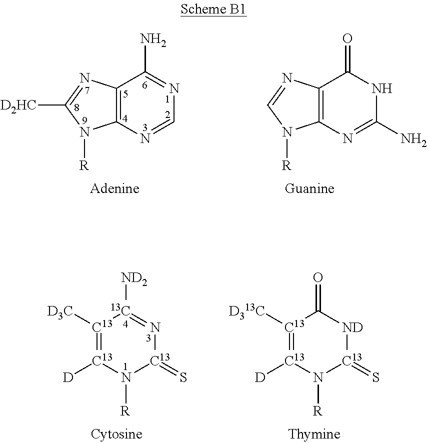

The mass modifications for the nucleotides of set B2 are at position C2′ in the sugar of the corresponding nucleosides, as shown in the structures below.

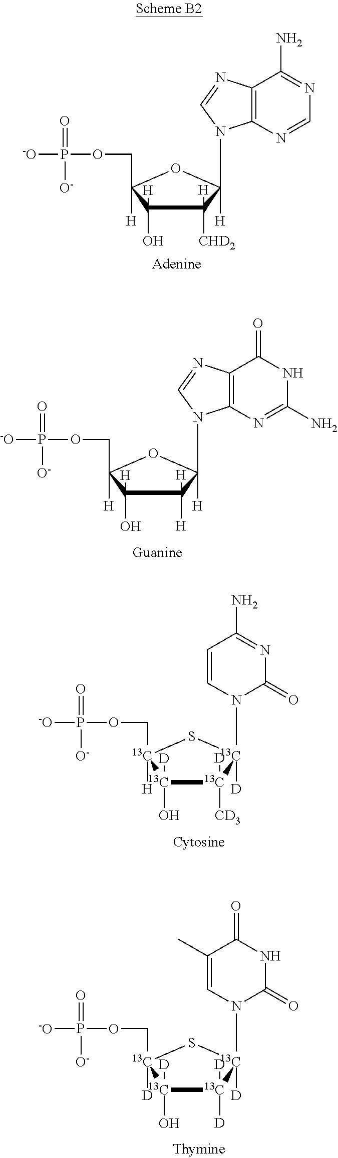

Set C

A third set (set C1, set C2) of mass-modified nucleotides are designed according to the following scheme. Each of adenine (A), thymine (T), guanine (G), and cytosine (C) are mass-modified such that their masses are substantially identical. Thymine has the molecular formula C5H6N2O2 and molar mass of about 126 g/mol. Two oxygen atoms in thymine are replaced with two sulfur atoms, which increases the molar mass by about 32 AMU to about 158 g/mol. Guanine has the molecular formula C5H5N5O and molar mass of about 151 g/mol, which is about 7 AMU less than the modified thymine nucleotide above. To increase the molar mass of guanine by about 7 AMU, 1) five carbon atoms are replaced with five carbon 13 isotopes (net gain of about 5 AMU), and 2) two hydrogen atoms are replaced with two deuterium atoms (net gain of about 2 AMU). Adenine has the molecular formula C5H5N5 and molar mass of about 135 g/mol, which is about 23 AMU less than the modified nucleotides above. To increase the molar mass of adenine by about 23 AMU, 1) one hydrogen atom is replaced with one methyl group having one carbon atom and three deuterium atoms (net gain of about 17 AMU), 2) one hydrogen atom is replaced with one deuterium atom (net gain of about 1 AMU), and 3) five carbon atoms are replaced with five carbon 13 isotopes (net gain of about 5 AMU). Cytosine has the molecular formula C4H5N3O and molar mass of about 111 g/mol, which is about 47 AMU less than the modified nucleotides above. To increase the molar mass of cytosine by about 47 AMU, 1) one oxygen atom is replaced with a sulfur atom (net gain of about 16 AMU), and 2) one hydrogen atom is replaced with one ethyl group having two carbon atoms, two hydrogen atoms and three deuterium atoms (net gain of about 31 AMU).

The mass modifications for the nucleotides of set C1 are in the purine base (at position C-8) or pyrimidine base (at position C-5), as shown in the structures below.

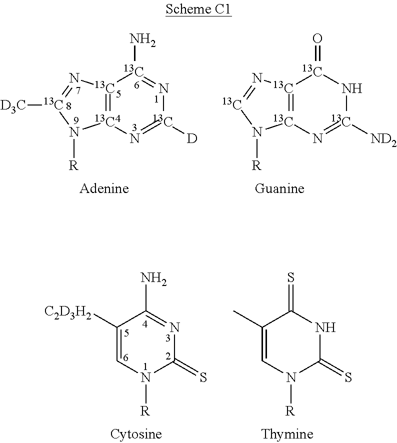

The mass modifications for the nucleotides of set C2 are at position C2′ in the sugar of the corresponding nucleosides, as shown in the structures below.

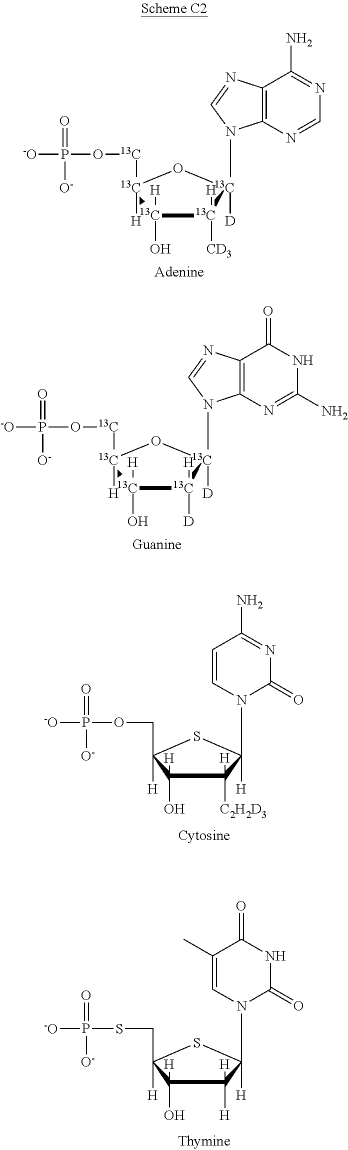

Set D

A fourth set (set D1, set D2) of mass-modified nucleotides are designed according to the following scheme. Each of adenine (A), thymine (T), guanine (G), and cytosine (C) are mass-modified such that their masses are substantially identical. Cytosine has the molecular formula C4H5N3O and molar mass of about 111 g/mol. An oxygen atom in cytosine is replaced with a selenium atom, which increases the molar mass by about 63 AMU to about 174 g/mol. Thymine has the molecular formula C5H6N2O2 and molar mass of about 126 g/mol, which is about 48 AMU less than the modified cytosine nucleotide above. To increase the molar mass of thymine by about 48 AMU, 1) two oxygen atoms are replaced with two sulfur atoms (net gain of about 32 AMU); and 2) one hydrogen atom is replaced with one methyl group having one carbon atom, one hydrogen atom, and two deuterium atoms (net gain of about 16 AMU). Adenine has the molecular formula C5H5N5 and molar mass of about 135 g/mol, which is about 39 AMU less than the modified nucleotides above. To increase the molar mass of adenine by about 39 AMU, 1) three hydrogen atoms are replaced with three deuterium atoms (net gain of about 3 AMU); and 2) one hydrogen atom is replaced with one ethyl group having two carbon 13 isotopes, and 5 deuterium atoms (net gain of about 36 AMU). Guanine has the molecular formula C5H5N5O and molar mass of about 151 g/mol, which is about 23 AMU less than the modified nucleotides above. To increase the molar mass of guanine by about 23 AMU, 1) one oxygen atom is replaced with one sulfur atom (net gain of about 16 AMU); 2) five carbon atoms are replaced with five carbon 13 isotopes (net gain of about 5 AMU); and 3) two hydrogen atoms are replaced with two deuterium atoms (net gain of about 2 AMU).

The mass modifications for the nucleotides of set D1 are in the purine base (at position C-8) or pyrimidine base (at position C-5), as shown in the structures below.

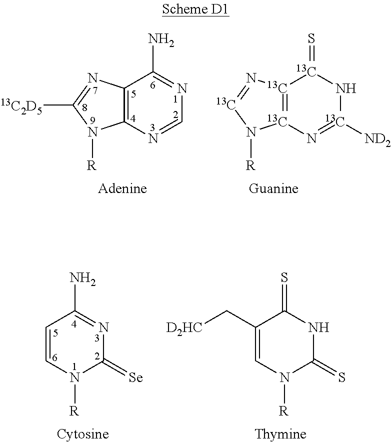

The mass modifications for the nucleotides of set D2 are at position C2′ in the sugar of the corresponding nucleosides, as shown in the structures below.

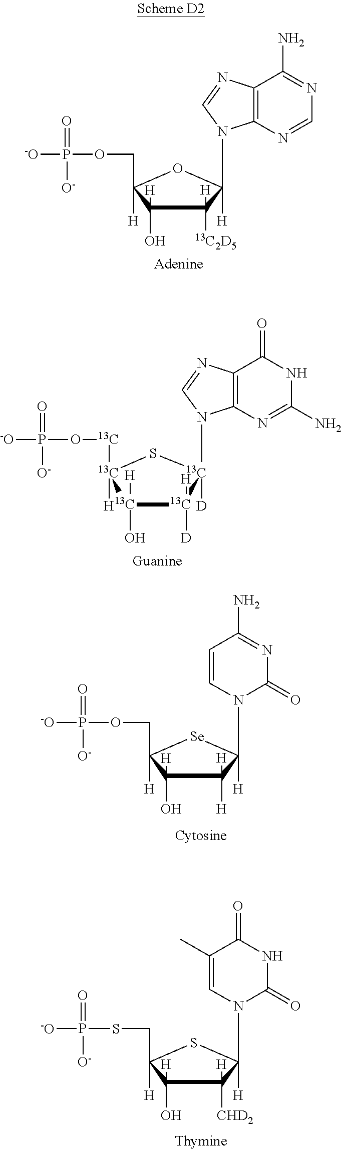

### Example 2: Detection of Trisomy 21 Using a Selective Capture Process and Length-Based Analysis of Nucleic Acid Fragments

Plasma samples containing circulating cell-free DNA obtained from pregnant females are tested for trisomy 21 using the following method.

Nucleic Acid Fragment Separation

A SURESELECT custom capture library is obtained from Agilent which includes a set of custom designed biotinylated capture RNAs. The capture RNAs are designed according to nucleotide sequences specific to chromosome 21 (test chromosome) and specific to chromosome 14 (reference chromosome) and are identified by Agilent's EARRAY web-based design tool. 100 independent capture RNAs are designed for each of chromosome 14 and chromosome 21. Single copy nucleotide sequences in the range of 40 to 60 base pairs that are unique to chromosome 14 or 21 are selected for the custom capture RNA design.

Sample nucleic acid, which is cell-free circulating plasma nucleic acid from a pregnant woman in the first trimester of pregnancy, is split into two tubes and incubated with either chromosome 21 capture RNA or chromosome 14 capture RNA for 24 hours at 65° C., according to the manufacturer's instruction. After hybridization, captured target fragments and captured reference fragments (collectively referred to as captured fragments) are selected by pulling down the biotinylated RNA/fragment hybrids by using streptavidin-coated magnetic beads (DYNAL DYNAMAG-2, Invitrogen, Carlsbad, Calif.), and purified with the MINELUTE PCR Purification Kit (Qiagen, Germantown, Md.). Capture RNA is digested and the remaining DNA fragments are amplified according to the manufacturer's instruction.

Length-Based Analysis

Samples containing separated nucleic acid fragments from above are hybridized under stringent hybridization conditions to probes comprising a set of the mass-modified nucleotides described in Example 1 and Biotin-11-dCTP (Jena Bioscience GmbH, Jena, Germany), which probes are designed according to nucleotide sequences specific to chromosome 21 and chromosome 14 described above, are longer than the DNA fragments to which they hybridize, and are 500 base pairs in length. In some embodiments, hybridization is performed overnight at 65° C. in 6×SSC and 1% SDS. In some embodiments, hybridization is performed overnight at 43° C. in 1.0M NaCl, 50 mM sodium phosphate buffer (pH 7.4), 1.0 mM EDTA, 2% (w/v) sodium dodecyl sulfate, 0.1% (w/v) gelatin, 50 μg/ml tRNA and 30% (v/v) formamide. Four 30 minute washes are performed at 55° C. in 1.2×SSC (1×SSC is 0.15M NaCl plus 0.015M sodium citrate), 10 mM sodium phosphate (pH 7.4), 1.0 mM EDTA and 0.5% (w/v) sodium dodecyl sulfate. After hybridization, unhybridized probe portions are digested using Exonuclease I (New England Biolabs, Ipswich, Mass.) and Phosphodiesterase II (Worthington Biochemical Corp., Lakewood, N.J.). The probe-fragment duplexes are denatured at 95° C. for two minutes and the probes are separated away from the fragments (i.e., pulled down) using streptavidin-coated magnetic beads (DYNAL DYNAMAG-2, Invitrogen, Carlsbad, Calif.), and purified with the MINELUTE PCR Purification Kit (Qiagen, Germantown, Md.). Trimmed, isolated and purified probes are measured for mass using MALDI mass spectrometry. Probe length, and thus corresponding fragment length, is extrapolated from the mass peaks for each probe length species by comparison to mass peaks for biotinylated mass-modified standards of known length.

Determination of Trisomy 21

The relative amount of each fragment length species is determined based on the amplitude of the mass peaks for each probe length species. Fragments of 150 base pairs or less are quantified for chromosome 14 and chromosome 21. Samples with substantially equal amounts of fragments from chromosome 14 and chromosome 21 are determined as euploid for chromosome 21. Samples with a statistically significantly higher amount of fragments from chromosome 21 versus chromosome 14 (e.g., 2% elevation in fragments from chromosome 21 versus chromosome 14) are determined as triploid for chromosome 21.

### Example 3: Detection of Trisomy 21 Using a Length-Based Analysis of Nucleic Acid Fragments

Plasma samples containing circulating cell-free DNA obtained from pregnant females are tested for trisomy 21 using the methods described in Example 2 above, with the exception that the biotinylated probes comprising mass-modified nucleotides also serve as capture oligonucleotide for the fragment separation step. Briefly, probes comprising mass-modified nucleotides described in Example 1 and Biotin-11-dCTP (Jena Bioscience GmbH, Jena, Germany) are designed according to nucleotide sequences specific to chromosome 21 (test chromosome) and specific to chromosome 14 (reference chromosome). 100 independent probes are designed for each of chromosome 14 and chromosome 21. Single copy nucleotide sequences in the range of 300 to 500 base pairs that are unique to chromosome 14 or 21 are selected for the probes. Probes also comprise non-human nucleotide sequences (e.g., sequences that do not hybridize to any sequence in the human genome) in the range of 50 to 100 base pairs at the 5′ and 3′ termini of each probe.

Sample nucleic acid, which is cell-free circulating plasma nucleic acid from a pregnant woman in the first trimester of pregnancy, is split into two tubes and incubated with either chromosome 21 probes or chromosome 14 probes for 24 hours in 0.5M sodium phosphate, 7% SDS at 65° C., followed by two washes at 0.2×SSC, 1% SDS at 65° C. After hybridization, captured target fragments and captured reference fragments (collectively referred to as captured fragments) are selected by pulling down the biotinylated probe/fragment duplexes by using streptavidin-coated magnetic beads (DYNAL DYNAMAG-2, Invitrogen, Carlsbad, Calif.), and purified with the MINELUTE PCR Purification Kit (Qiagen, Germantown, Md.).

Unhybridized probe portions of the probe/fragment duplexes above are digested using Exonuclease I (New England Biolabs, Ipswich, Mass.) and Phosphodiesterase II (Worthington Biochemical Corp., Lakewood, N.J.). The probe-fragment duplexes are denatured at 95° C. for two minutes and the trimmed probes are separated away from the fragments (i.e., pulled down) using streptavidin-coated magnetic beads (DYNAL DYNAMAG-2, Invitrogen, Carlsbad, Calif.), and purified with the MINELUTE PCR Purification Kit (Qiagen, Germantown, Md.). Trimmed, isolated and purified probes are measured for mass using MALDI mass spectrometry. Probe length, and thus corresponding fragment length, is extrapolated from the mass peaks for each probe length species by comparison to mass peaks for biotinylated mass-modified standards of known length. The presence or absence of trisomy 21 is determined using the method described in Example 2.

### Example 4: Measurement of Nucleic Acid Fragment Length

Nucleic acid fragment length is determined using the following method.

Nucleic acid fragments from a sample comprising a mixture of nucleic acid fragments of various lengths are ligated to a universal priming site nucleotide sequence comprising Biotin-11-dCTP (Jena Bioscience GmbH, Jena, Germany) using T4 DNA ligase (New England Biolabs, Ipswich, Mass.) according to manufacturer's instruction. Ligated fragments are denatured at 95° C. for two minutes, generating single-stranded ligated fragments. Universal primers are annealed to the single-stranded ligated fragments at 65° C. for one minute, and the primed fragments are extended with modified nucleotides from a set of mass-modified nucleotides described in Example 1 using DNA Polymerase I, Large (Klenow) Fragment (New England Biolabs, Ipswich, Mass.) at 72° C. for one minute. Copy-fragment duplexes are denatured at 95° C. for two minutes and the original fragments are separated away from the copy strands (i.e., pulled down) using streptavidin-coated magnetic beads (DYNAL DYNAMAG-2, Invitrogen, Carlsbad, Calif.), and the copy strands are purified with the MINELUTE PCR Purification Kit (Qiagen, Germantown, Md.). Copy strands comprising mass-modified nucleotides are measured for mass using MALDI mass spectrometry. Copy length, and thus corresponding fragment length, is extrapolated from the mass peaks for each copy length species by comparison to mass peaks for mass-modified standards of known length and subtraction of universal primer mass.

### Example 5: Examples of Embodiments

A1. A composition comprising four nucleotide species, wherein the nucleotide species have substantially identical separation properties when separated by a mass-sensitive process.

A1.1 The composition of embodiment A1, wherein polynucleotides having an equal total number of the nucleotide species have substantially identical separation properties when separated by a mass-sensitive process.

A2. The composition of embodiment A1 or A1.1, wherein at least three of the nucleotide species are mass-modified.

A3. The composition of embodiment A2, wherein the nucleotide species have substantially identical mass.

A4. The composition of any one of embodiments A1 to A3, wherein the nucleotide species each are capable of hybridizing to one of adenine, thymine, cytosine and guanine in a polynucleotide, wherein the adenine, thymine, cytosine and guanine are not mass-modified.

A5. The composition of any one of embodiments A1 to A4, wherein the nucleotide species are capable of forming phosphodiester bonds when polymerized.

A5.1 The composition of any one of embodiments A1 to A5, wherein the nucleotide species are capable of being polymerized by a polymerase on a nucleic acid template.

A6. The composition of any one of embodiments A2 to A5.1, wherein each mass-modified nucleotide species comprises one or more mass modifiers.

A7. The composition of any one of embodiments A2 to A6, wherein each mass-modified nucleotide species comprises one or more isotopes.

A8. The composition of embodiment A7, wherein the one or more isotopes are one or more stable isotopes.

A9. The composition of any one of embodiments A2 to A8, wherein each mass-modified nucleotide species comprises one or more isotopes and one or more other mass modifiers.

A10. The composition of embodiment A7, A8 or A9, wherein the one or more isotopes comprise a hydrogen isotope.

A11. The composition of embodiment A10, wherein the hydrogen isotope is deuterium.

A12. The composition of embodiment A7, A8 or A9, wherein the one or more isotopes comprise a nitrogen isotope.

A12.1 The composition of embodiment A12, wherein the nitrogen isotope is nitrogen-15.

A13. The composition of embodiment A7, A8 or A9, wherein the one or more isotopes comprise an oxygen isotope.

A13.1 The composition of embodiment A13, wherein the oxygen isotope is oxygen-17 or oxygen-18.

A14. The composition of embodiment A7, A8 or A9, wherein the one or more isotopes comprise a carbon isotope.

A14.1 The composition of embodiment A14, wherein the carbon isotope is carbon-13.

B1. A method for determining length of a nucleic acid fragment, comprising:
a) contacting, under annealing conditions, a nucleic acid fragment with a probe, which probe:
   (i) comprises at least two nucleotide species which have substantially identical separation properties, and
   (ii) is longer than the nucleic acid fragment to which it anneals,
thereby generating a fragment-probe species comprising one or more unhybridized probe portions;
b) removing the one or more unhybridized probe portions from the fragment-probe species, thereby generating a trimmed probe; and
c) determining the length of the trimmed probe, thereby determining the length of the nucleic acid fragment.

B1.1 A method for determining lengths of nucleic acid fragments in a mixture of nucleic acid fragments having different lengths, comprising:
a) contacting, under annealing conditions, nucleic acid fragments with a plurality of probes, which probes:
   (i) comprise at least two nucleotide species which have substantially identical separation properties, and
   (ii) are longer than the nucleic acid fragments to which they anneal, thereby generating fragment-probe species comprising unhybridized probe portions;
b) removing the unhybridized probe portions from the fragment-probe species, thereby generating trimmed probes; and
c) determining lengths of the trimmed probes, thereby determining the lengths of the nucleic acid fragments.

B2. The method of embodiment B1 or B1.1, wherein the probe comprises at least one mass-modified nucleotide species.

B3. The method of embodiment B2, wherein the probe comprises at least two mass-modified nucleotide species.

B4. The method of embodiment B3, wherein the probe comprises at least three mass-modified nucleotide species.

B5. The method of embodiment B4, wherein the probe comprises at least four mass-modified nucleotide species.

B6. The method of embodiment B3, B4 or B5, wherein the probe comprises at least three nucleotide species of substantially identical mass.

B7. The method of embodiment B6, wherein the probe comprises at least four nucleotide species of substantially identical mass.

B8. The method of embodiment B7, wherein all nucleotide species in the probe are of substantially identical mass.

B9. The method of embodiment B3, wherein the probe comprises a first set of nucleotide species having substantially identical mass and a second set of nucleotide species having substantially identical mass, wherein the mass of the first set is different than the mass of the second set.

B10. The method of embodiment B9, wherein nucleotide species of the first set are purines, derivatives thereof or combinations thereof and nucleotide species of the second set are pyrimidines, derivatives thereof or combinations thereof.

B11. The method of any one of embodiments B2 to B10, wherein the mass-modified nucleotide species are joined by phosphodiester bonds in the probes.

B12. The method of any one of embodiments B2 to B11, wherein each mass-modified nucleotide species is capable of hybridizing to one of adenine, thymine, cytosine and guanine in a polynucleotide, wherein the adenine, thymine, cytosine and guanine are not mass-modified.

B13. The method of any one of embodiments B2 to B12, wherein each mass-modified nucleotide species comprises one or more mass modifiers.

B14. The method of any one of embodiments B2 to B13, wherein each mass-modified nucleotide species comprises one or more isotopes.

B15. The method of embodiment B14, wherein the one or more isotopes are one or more stable isotopes.

B16. The method of any one of embodiments B2 to B15, wherein each mass-modified nucleotide species comprises one or more isotopes and one or more other mass modifiers.

B17. The method of embodiment B14, B15 or B16, wherein the one or more isotopes comprise a hydrogen isotope.

B18. The method of embodiment B17, wherein the hydrogen isotope is deuterium.

B19. The method of embodiment B14, B15 or B16, wherein the one or more isotopes comprise a nitrogen isotope.

B19.1 The method of embodiment B19, wherein the nitrogen isotope is nitrogen-15.

B20. The method of embodiment B14, B15 or B16, wherein the one or more isotopes comprise an oxygen isotope.

B20.1 The method of embodiment B20, wherein the oxygen isotope is oxygen-17 or oxygen-18.

B21. The method of embodiment B14, B15 or B16, wherein the one or more isotopes comprise a carbon isotope.

B21.1 The method of embodiment B21, wherein the carbon isotope is carbon-13.

B22. The method of any one of embodiments B1 to B21.1, wherein the determining the lengths of the trimmed probes comprises use of a mass sensitive process.

B23. The method of embodiment B22, wherein the mass sensitive process comprises mass spectrometry.

B24. The method of embodiment B22, wherein the mass sensitive process comprises electrophoresis.

B24.1 The method of embodiment B22, wherein the mass sensitive process does not comprise electrophoresis.

B25. The method of any one of embodiments B1 to B24.1, with the proviso that the nucleotide sequences of the nucleic acid fragments are not determined.

C1. A method for detecting the presence or absence of a genetic variation comprising:
(a) contacting under annealing conditions target fragments and reference fragments from a nucleic acid sample with a plurality of probes that can anneal to the fragments, which probes (1) comprise at least two nucleotide species which have substantially identical separation properties, and (2) are longer than the fragments to which they anneal, thereby generating target-probe species and reference-probe species comprising unhybridized probe portions;
(b) separating the target-probe species reference-probe species from the nucleic acid sample;
(c) removing the unhybridized probe portions of the target-probe species and the reference-probe species, thereby generating trimmed probes;
(d) determining lengths of the trimmed probes, thereby determining the lengths of the target fragments and reference fragments;
(e) quantifying the amount of at least one target fragment length species and at least one reference fragment length species; and
(f) providing an outcome determinative of the presence or absence of a genetic variation from the quantification in (e), with the proviso that the outcome is provided without determining nucleotide sequences of the target fragments and the reference fragments.

C1.1 A method for detecting the presence or absence of a genetic variation comprising:
(a) separating target fragments and reference fragments from a nucleic acid sample based on nucleotide sequences in the target fragments and the reference fragments and substantially not in other fragments in the sample, thereby generating separated fragments comprising separated target fragments and separated reference fragments;
(b) determining lengths of the separated target fragments and separated reference fragments by a process comprising:
   i) contacting under annealing conditions the separated fragments with a plurality of probes that can anneal to the separated fragments, which probes (1) comprise at least two nucleotide species which have substantially identical separation properties, and (2) are longer than the separated fragments to which they anneal, thereby generating target-probe species and reference-probe species comprising unhybridized probe portions;
   ii) removing the unhybridized probe portions of the target-probe species and the reference-probe species, thereby generating trimmed probes; and
   iii) determining lengths of the trimmed probes, thereby determining the lengths of the separated target fragments and separated reference fragments;
(c) quantifying the amount of at least one separated target fragment length species and at least one separated reference fragment length species; and
(d) providing an outcome determinative of the presence or absence of a genetic variation from the quantification in (c), with the proviso that the outcome is provided without determining nucleotide sequences of the target fragments and the reference fragments.

C2. The method of embodiment C1 or C1.1, wherein the probe comprises at least one mass-modified nucleotide species.

C3. The method of embodiment C2, wherein the probe comprises at least two mass-modified nucleotide species.

C4. The method of embodiment C3, wherein the probe comprises at least three mass-modified nucleotide species.

C5. The method of embodiment C4, wherein the probe comprises at least four mass-modified nucleotide species.

C6. The method of embodiment C3, C4 or C5, wherein the probe comprises at least three nucleotide species of substantially identical mass.

C7. The method of embodiment C6, wherein the probe comprises at least four nucleotide species of substantially identical mass.

C8. The method of embodiment C7, wherein all nucleotide species in the probe are of substantially identical mass.

C9. The method of embodiment C3, wherein the probe comprises a first set of nucleotide species having substantially equal mass and a second set of nucleotide species having substantially equal mass, wherein the mass of the first set is different than the mass of the second set.

C10. The method of embodiment C9, wherein nucleotide species of the first set are purines, derivatives thereof or combinations thereof and nucleotide species of the second set are pyrimidines, derivatives thereof or combinations thereof.

C11. The method of any one of embodiments C2 to C10, wherein the mass-modified nucleotide species are joined by phosphodiester bonds in the probes.

C12. The method of any one of embodiments C2 to C11, wherein each mass-modified nucleotide species is capable of hybridizing to one of adenine, thymine, cytosine and guanine in a polynucleotide, wherein the adenine, thymine, cytosine and guanine are not mass-modified.

C13. The method of any one of embodiments C2 to C12, wherein each mass-modified nucleotide species comprises one or more mass modifiers.

C14. The method of any one of embodiments C2 to C13, wherein each mass-modified nucleotide species comprises one or more isotopes.

C15. The method of embodiment C14, wherein the one or more isotopes are one or more stable isotopes.

C16. The method of any one of embodiments C2 to C15, wherein each mass-modified nucleotide species comprises one or more isotopes and one or more other mass modifiers.

C17. The method of embodiment C14, C15 or C16, wherein the one or more isotopes comprise a hydrogen isotope.

C18. The method of embodiment C17, wherein the hydrogen isotope is deuterium.

C19. The method of embodiment C14, C15 or C16, wherein the one or more isotopes comprise a nitrogen isotope.

C19.1 The method of embodiment C19, wherein the nitrogen isotope is nitrogen-15.

C20. The method of embodiment C14, C15 or C16, wherein the one or more isotopes comprise an oxygen isotope.

C20.1 The method of embodiment C20, wherein the oxygen isotope is oxygen-17 or oxygen-18.

C21. The method of embodiment C14, C15 or C16, wherein the one or more isotopes comprise a carbon isotope.

C21.1 The method of embodiment C21, wherein the carbon isotope is carbon-13.

C22. The method of any one of embodiments C1 to C21.1, wherein the determining the lengths of the trimmed probes comprises use of a mass sensitive process.

C23. The method of embodiment C22, wherein the mass sensitive process comprises mass spectrometry.

C24. The method of embodiment C22, wherein the mass sensitive process comprises electrophoresis.

C24.1 The method of embodiment C22, wherein the mass sensitive process does not comprise electrophoresis.

C25. The method of any one of embodiments C1 to C24.1, wherein the number of fragments in the sample is determined for at least one target fragment length species and at least one reference fragment length species.

C26. The method of any one of embodiments C1.1 to C25, wherein the target fragments and reference fragments are separated using a selective nucleic acid capture process.

C27. The method of embodiment C26, wherein the selective nucleic acid capture process comprises use of a solid phase array.

C28. The method of any one of embodiments C1 to C27, further comprising isolating a sample from a subject.

C29. The method of embodiment C28, wherein the sample is from a pregnant female.

C30. The method of embodiment C28 or C29, wherein the sample is blood.

C31. The method of embodiment C28 or C29, wherein the sample is urine.

C32. The method of embodiment C28 or C29, wherein the sample is saliva.

C33. The method of embodiment C28 or C29, wherein the sample is a cervical swab.

C34. The method of embodiment C30, wherein the sample is serum.

C35. The method of embodiment C30, wherein the sample is plasma.

C36. The method of any one of embodiments C28 to C35, comprising isolating nucleic acid from the sample.

C37. The method of embodiment C36, wherein the nucleic acid in the sample is circulating cell-free nucleic acid.

C38. The method of any one of embodiments C1 to C37, wherein the genetic variation is a fetal aneuploidy.

C39. The method of embodiment C38, wherein the fetal aneuploidy is trisomy 13.

C40. The method of embodiment C39, wherein the fetal aneuploidy is trisomy 18.

C41. The method of embodiment C40, wherein the fetal aneuploidy is trisomy 21.

C42. The method of embodiment C39, wherein the target nucleic acid fragments are from chromosome 13.

C43. The method of embodiment C40, wherein the target nucleic acid fragments are from chromosome 18.

C44. The method of embodiment C41, wherein the target nucleic acid fragments are from chromosome 21.

C45. The method of any one of embodiments C29 to C44, further comprising determining the fraction of fetal nucleic acid in the sample and providing the outcome based in part on the fraction.

D1. A method for determining length of a nucleic acid fragment, comprising:
a) generating a complementary copy of the nucleic acid fragment, which fragment copy comprises at least two nucleotide species which have substantially identical separation properties; and
b) determining the length of the fragment copy, thereby determining the length of the nucleic acid fragment.

D1.1 A method for determining length of a nucleic acid fragment, comprising:
a) ligating a priming site to the nucleic acid fragment, thereby generating a ligated nucleic acid fragment;
b) contacting, under annealing conditions, the ligated nucleic acid fragment with a primer which is capable of hybridizing to the primer site in (a);
c) extending the primer with a set of nucleotides, which set comprises at least two nucleotide species which have substantially identical separation properties, thereby generating a complementary copy of the fragment comprising modified nucleotides; and
d) determining the length of the fragment copy, thereby determining the length of the nucleic acid fragment.

D1.2 A method for determining lengths of nucleic acid fragments in a mixture of nucleic acid fragments having different lengths, comprising:
a) ligating priming sites to the nucleic acid fragments, thereby generating ligated nucleic acid fragments;
b) contacting, under annealing conditions, the ligated nucleic acid fragments with primers which are capable of hybridizing to the priming sites in (a);
c) extending the primers with a set of nucleotides, which set comprises at least two nucleotide species which have substantially identical separation properties, thereby generating complementary copies of the fragments comprising modified nucleotides; and
d) determining the lengths of the fragment copies, thereby determining the lengths of the nucleic acid fragments.

D2. The method of embodiment D1, D1.1 or D1.2, wherein the fragment copy comprises at least one mass-modified nucleotide species.

D3. The method of embodiment D2, wherein the fragment copy comprises at least two mass-modified nucleotide species.

D4. The method of embodiment D3, wherein the fragment copy comprises at least three mass-modified nucleotide species.

D5. The method of embodiment D4, wherein the fragment copy comprises at least four mass-modified nucleotide species.

D6. The method of embodiment D3, D4 or D5, wherein the fragment copy comprises at least three nucleotide species of substantially identical mass.

D7. The method of embodiment D6, wherein the fragment copy comprises at least four nucleotide species of substantially identical mass.

D8. The method of embodiment D7, wherein all nucleotide species in the fragment copy are of substantially identical mass.

D9. The method of embodiment D3, wherein the fragment copy comprises a first set of nucleotide species having substantially identical mass and a second set of nucleotide species having substantially identical mass, wherein the mass of the first set is different than the mass of the second set.

D10. The method of embodiment D9, wherein nucleotide species of the first set are purines, derivatives thereof or combinations thereof and nucleotide species of the second set are pyrimidines, derivatives thereof or combinations thereof.

D11. The method of any one of embodiments D2 to D10, wherein the mass-modified nucleotide species are joined by phosphodiester bonds in the fragment copy.

D11.1 The method of any one of embodiments D2 to D11, wherein the mass-modified nucleotide species are capable of polymerizing on a nucleic acid template.

D12. The method of any one of embodiments D2 to D11.1, wherein each mass-modified nucleotide species is capable of hybridizing to one of adenine, thymine, cytosine and guanine in a polynucleotide, wherein the adenine, thymine, cytosine and guanine are not mass-modified.

D13. The method of any one of embodiments D2 to D12, wherein each mass-modified nucleotide species comprises one or more mass modifiers.

D14. The method of any one of embodiments D2 to D13, wherein each mass-modified nucleotide species comprises one or more isotopes.

D15. The method of embodiment D14, wherein the one or more isotopes are one or more stable isotopes.

D16. The method of any one of embodiments D2 to D15, wherein each mass-modified nucleotide species comprises one or more isotopes and one or more other mass modifiers.

D17. The method of embodiment D14, D15 or D16, wherein the one or more isotopes comprise a hydrogen isotope.

D18. The method of embodiment D17, wherein the hydrogen isotope is deuterium.

D19. The method of embodiment D14, D15 or D16, wherein the one or more isotopes comprise a nitrogen isotope.

D19.1 The method of embodiment D19, wherein the nitrogen isotope is nitrogen-15.

D20. The method of embodiment D14, D15 or D16, wherein the one or more isotopes comprise an oxygen isotope.

D20.1 The method of embodiment D20, wherein the oxygen isotope is oxygen-17 or oxygen-18.

D21. The method of embodiment D14, D15 or D16, wherein the one or more isotopes comprise a carbon isotope.

D21.1 The method of embodiment D21, wherein the carbon isotope is carbon-13.

D22. The method of any one of embodiments D1 to D21.1, wherein the determining the lengths of the fragment copies comprises use of a mass sensitive process.

D23. The method of embodiment D22, wherein the mass sensitive process comprises mass spectrometry.

D24. The method of embodiment D22, wherein the mass sensitive process comprises electrophoresis.

D24.1 The method of embodiment D22, wherein the mass sensitive process does not comprise electrophoresis.

D25. The method of any one of embodiments D1 to D24.1, with the proviso that the nucleotide sequences of the nucleic acid fragments are not determined.

E1. A method for detecting the presence or absence of a genetic variation comprising:
(a) separating target fragments and reference fragments from a nucleic acid sample based on nucleotide sequences in the target fragments and the reference fragments and substantially not in other fragments in the sample, thereby generating separated fragments comprising separated target fragments and separated reference fragments;
(b) determining lengths of the separated target fragments and separated reference fragments by a process comprising:
   i) generating complementary copies of the of the separated target fragments and separated reference fragments, wherein each fragment copy comprises at least two nucleotide species which have substantially identical separation properties; and
   ii) determining the lengths of the fragment copies, thereby determining the lengths of the separated target fragments and separated reference fragments
(c) quantifying the amount of at least one separated target fragment length species and at least one separated reference fragment length species; and
(d) providing an outcome determinative of the presence or absence of a genetic variation from the quantification in (c).

E1.1 The method of embodiment E1, with the proviso that the outcome is provided without determining nucleotide sequences of the target fragments and the reference fragments

E2. The method of embodiment E1 or E1.1, wherein the fragment copy comprises at least one mass-modified nucleotide species.

E3. The method of embodiment E2, wherein the fragment copy comprises at least two mass-modified nucleotide species.

E4. The method of embodiment E3, wherein the fragment copy comprises at least three mass-modified nucleotide species.

E5. The method of embodiment E4, wherein the fragment copy comprises at least four mass-modified nucleotide species.

E6. The method of embodiment E3, E4 or E5, wherein the fragment copy comprises at least three nucleotide species of substantially identical mass.

E7. The method of embodiment E6, wherein the fragment copy comprises at least four nucleotide species of substantially identical mass.

E8. The method of embodiment E7, wherein all nucleotide species in the fragment copy are of substantially identical mass.

E9. The method of embodiment E3, wherein the fragment copy comprises a first set of nucleotide species having substantially equal mass and a second set of nucleotide species having substantially equal mass, wherein the mass of the first set is different than the mass of the second set.

E10. The method of embodiment E9, wherein nucleotide species of the first set are purines, derivatives thereof or combinations thereof and nucleotide species of the second set are pyrimidines, derivatives thereof or combinations thereof.

E11. The method of any one of embodiments E2 to E10, wherein the mass-modified nucleotide species are joined by phosphodiester bonds in the fragment copy.

E11.1 The method of any one of embodiments E2 to E11, wherein the mass-modified nucleotide species are capable of polymerizing on a nucleic acid template.

E12. The method of any one of embodiments E2 to E11.1, wherein each mass-modified nucleotide species is capable of hybridizing to one of adenine, thymine, cytosine and guanine in a polynucleotide, wherein the adenine, thymine, cytosine and guanine are not mass-modified.

E13. The method of any one of embodiments E2 to E12, wherein each mass-modified nucleotide species comprises one or more mass modifiers.

E14. The method of any one of embodiments E2 to E13, wherein each mass-modified nucleotide species comprises one or more isotopes.

E15. The method of embodiment E14, wherein the one or more isotopes are one or more stable isotopes.

E16. The method of any one of embodiments E2 to E15, wherein each mass-modified nucleotide species comprises one or more isotopes and one or more other mass modifiers.

E17. The method of embodiment E14, E15 or E16, wherein the one or more isotopes comprise a hydrogen isotope.

E18. The method of embodiment E17, wherein the hydrogen isotope is deuterium.

E19. The method of embodiment E14, E15 or E16, wherein the one or more isotopes comprise a nitrogen isotope.

E19.1 The method of embodiment E19, wherein the nitrogen isotope is nitrogen-15.

E20. The method of embodiment E14, E15 or E16, wherein the one or more isotopes comprise an oxygen isotope.

E20.1 The method of embodiment E20, wherein the oxygen isotope is oxygen-17 or oxygen-18.

E21. The method of embodiment E14, E15 or E16, wherein the one or more isotopes comprise a carbon isotope.

E21.1 The method of embodiment E21, wherein the carbon isotope is carbon-13.

E22. The method of any one of embodiments E1 to E21.1, wherein the determining the lengths of the fragment copies comprises use of a mass sensitive process.

E23. The method of embodiment E22, wherein the mass sensitive process comprises mass spectrometry.

E24. The method of embodiment E22, wherein the mass sensitive process comprises electrophoresis.

E24.1 The method of embodiment E22, wherein the mass sensitive process does not comprise electrophoresis.

E25. The method of any one of embodiments E1 to E24.1, wherein the number of fragments in the sample is determined for at least one target fragment length species and at least one reference fragment length species.

E26. The method of any one of embodiments E1 to E25, wherein the target fragments and reference fragments are separated using a selective nucleic acid capture process.

E27. The method of embodiment E26, wherein the selective nucleic acid capture process comprises use of a solid phase array.

E28. The method of any one of embodiments E1 to E27, further comprising isolating a sample from a subject.

E29. The method of embodiment E28, wherein the sample is from a pregnant female.

E30. The method of embodiment E28 or E29, wherein the sample is blood.

E31. The method of embodiment E28 or E29, wherein the sample is urine.

E32. The method of embodiment E28 or E29, wherein the sample is saliva.

E33. The method of embodiment E28 or E29, wherein the sample is a cervical swab.

E34. The method of embodiment E30, wherein the sample is serum.

E35. The method of embodiment E30, wherein the sample is plasma.

E36. The method of any one of embodiments E28 to E35, comprising isolating nucleic acid from the sample.

E37. The method of embodiment E36, wherein the nucleic acid in the sample is circulating cell-free nucleic acid.

E38. The method of any one of embodiments E1 to E37, wherein the genetic variation is a fetal aneuploidy.

E39. The method of embodiment E38, wherein the fetal aneuploidy is trisomy 13.

E40. The method of embodiment E39, wherein the fetal aneuploidy is trisomy 18.

E41. The method of embodiment E40, wherein the fetal aneuploidy is trisomy 21.

E42. The method of embodiment E39, wherein the target nucleic acid fragments are from chromosome 13.

E43. The method of embodiment E40, wherein the target nucleic acid fragments are from chromosome 18.

E44. The method of embodiment E41, wherein the target nucleic acid fragments are from chromosome 21.

E45. The method of any one of embodiments E29 to E44, further comprising determining the fraction of fetal nucleic acid in the sample and providing the outcome based in part on the fraction.

F1. A method of generating a complementary copy of a nucleic acid fragment comprising contacting under polymerization conditions a nucleic acid fragment with a composition comprising four nucleotide species, wherein the nucleotide species have substantially identical separation properties when separated by a mass-sensitive process, thereby generating a complementary copy of the nucleic acid fragment.

F1.1 The method of embodiment F1, wherein polynucleotides having an equal total number of the nucleotide species have substantially identical separation properties when separated by a mass-sensitive process.

F2. The method of embodiment F1 or F1.1, wherein at least three of the nucleotide species are mass-modified.

F3. The method of embodiment F2, wherein the nucleotide species have substantially identical mass.

F4. The method of any one of embodiments F1 to F3, wherein the nucleotide species each are capable of hybridizing to one of adenine, thymine, cytosine and guanine in a polynucleotide, wherein the adenine, thymine, cytosine and guanine are not mass-modified.

F5. The method of any one of embodiments F1 to F4, wherein the nucleotide species are capable of forming phosphodiester bonds when polymerized.

F5.1 The method of any one of embodiments F1 to F5, wherein the nucleotide species are capable of being polymerized by a polymerase on a nucleic acid template.

F6. The method of any one of embodiments F2 to F5.1, wherein each mass-modified nucleotide species comprises one or more mass modifiers.

F7. The method of any one of embodiments F2 to F6, wherein each mass-modified nucleotide species comprises one or more isotopes.

F8. The method of embodiment F7, wherein the one or more isotopes are one or more stable isotopes.

F9. The method of any one of embodiments F2 to F8, wherein each mass-modified nucleotide species comprises one or more isotopes and one or more other mass modifiers.

F10. The method of embodiment F7, F8 or F9, wherein the one or more isotopes comprise a hydrogen isotope.

F11. The method of embodiment F10, wherein the hydrogen isotope is deuterium.

F12. The method of embodiment F7, F8 or F9, wherein the one or more isotopes comprise a nitrogen isotope.

F12.1 The method of embodiment F12, wherein the nitrogen isotope is nitrogen-15.

F13. The method of embodiment F7, F8 or F9, wherein the one or more isotopes comprise an oxygen isotope.

F13.1 The method of embodiment F13, wherein the oxygen isotope is oxygen-17 or oxygen-18.

F14. The method of embodiment F7, F8 or F9, wherein the one or more isotopes comprise a carbon isotope.

F14.1 The method of embodiment F14, wherein the carbon isotope is carbon-13.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents.

Modifications may be made to the foregoing without departing from the basic aspects of the technology. Although the technology has been described in substantial detail with reference to one or more specific embodiments, those of ordinary skill in the art will recognize that changes may be made to the embodiments specifically disclosed in this application, yet these modifications and improvements are within the scope and spirit of the technology.

The technology illustratively described herein suitably may be practiced in the absence of any element(s) not specifically disclosed herein. Thus, for example, in each instance herein any of the terms “comprising,” “consisting essentially of,” and “consisting of” may be replaced with either of the other two terms. The terms and expressions which have been employed are used as terms of description and not of limitation, and use of such terms and expressions do not exclude any equivalents of the features shown and described or portions thereof, and various modifications are possible within the scope of the technology claimed. The term “a” or “an” can refer to one of or a plurality of the elements it modifies (e.g., “a reagent” can mean one or more reagents) unless it is contextually clear either one of the elements or more than one of the elements is described. The term “about” as used herein refers to a value within 10% of the underlying parameter (i.e., plus or minus 10%), and use of the term “about” at the beginning of a string of values modifies each of the values (i.e., “about 1, 2 and 3” refers to about 1, about 2 and about 3). For example, a weight of “about 100 grams” can include weights between 90 grams and 110 grams. Further, when a listing of values is described herein (e.g., about 50%, 60%, 70%, 80%, 85% or 86%) the listing includes all intermediate and fractional values thereof (e.g., 54%, 85.4%). Thus, it should be understood that although the present technology has been specifically disclosed by representative embodiments and optional features, modification and variation of the concepts herein disclosed may be resorted to by those skilled in the art, and such modifications and variations are considered within the scope of this technology.

Certain embodiments of the technology are set forth in the claim(s) that follow(s).

What is claimed is:

1. An oligonucleotide probe comprising:
at least two nucleotide species that are indistinguishable from each other by mass spectrometric analysis;
at least one mass-modified nucleotide species;
a first portion comprising a polynucleotide sequence complementary to a polynucleotide sequence of a strand of a target nucleic acid fragment, capable of hybridizing with the nucleic acid fragment along its entire length, wherein the length of a target nucleic acid fragment can be used to identify the fragment;
one or more second portions at a 5′ and/or 3′ end of the first portion of the probe comprising polynucleotide sequences that are not complementary to the polynucleotide sequence of a strand of a target nucleic acid fragment, that can be removed from the first portion when the first portion is hybridized with the target nucleic acid fragment, to produce a trimmed probe;
wherein (i) a trimmed probe has a length that corresponds with the length of the target nucleic acid fragment; (ii) the length of a trimmed probe can be used to identify the trimmed probe; (iii) the trimmed probe length can be determined by measuring the mass of the trimmed probe using mass spectrometry and (iv) determining the presence or absence of a trimmed probe thereby determines the presence or absence of the target nucleic acid fragment.

2. The oligonucleotide probe of claim 1, wherein the nucleotide species each have an identical mass.

3. The oligonucleotide probe of claim 2, wherein the nucleotide species each have a molar mass with a difference of 1 atomic mass unit (AMU).

4. The oligonucleotide probe of claim 2, wherein the nucleotide species each have a molar mass with a difference of 0.1 atomic mass unit (AMU).

5. The oligonucleotide probe of claim 2, wherein the nucleotide species each have a molar mass with a difference of 0.01 atomic mass unit (AMU).

6. The oligonucleotide probe of claim 2, wherein the nucleotide species each have a molar mass with a difference 0.001 atomic mass unit (AMU).

7. The oligonucleotide probe of claim 1, wherein the probe comprises at least two mass-modified nucleotide species.

8. The oligonucleotide probe of claim 7, wherein the probe comprises at least three mass-modified nucleotide species.

9. The oligonucleotide probe of claim 8, wherein the probe comprises at least four mass-modified nucleotide species.

10. The oligonucleotide probe of claim 7, wherein the probe comprises at least three nucleotide species of identical mass.

11. The oligonucleotide probe of claim 8, wherein the probe comprises at least four nucleotide species of identical mass.

12. The oligonucleotide probe of claim 11, wherein all nucleotide species in the probe are of identical mass.

13. The oligonucleotide probe of claim 7, wherein the probe comprises a first set of nucleotide species having identical mass and a second set of nucleotide species having identical mass, wherein the mass of the first set is different than the mass of the second set.

14. The oligonucleotide probe of claim 13, wherein nucleotide species of the first set are purines, derivatives thereof or combinations thereof and nucleotide species of the second set are pyrimidines, derivatives thereof or combinations thereof.

15. The oligonucleotide probe of claim 1, wherein the mass-modified nucleotide species are joined by phosphodiester bonds in the probes.

16. The oligonucleotide probe of claim 1, wherein each mass-modified nucleotide species is capable of hybridizing to one of adenine, thymine, cytosine and guanine in a polynucleotide, wherein the adenine, thymine, cytosine and guanine are not mass-modified.

17. The oligonucleotide probe of claim 1, wherein each mass-modified nucleotide species comprises one or more mass modifiers.

18. The oligonucleotide probe of claim 1, wherein each mass-modified nucleotide species comprises one or more isotopes.

19. The oligonucleotide probe of claim 18, wherein the one or more isotopes are one or more stable isotopes.

20. The oligonucleotide probe of claim 1, wherein each mass-modified nucleotide species comprises one or more isotopes and one or more other mass modifiers.

21. The oligonucleotide probe of claim 1, wherein one or more second portions of the probe comprise non-human nucleotide sequences.
